SEQUENCE LISTING (252 sequences)

SEQ ID NO: 1 - DNA, 22 bp, Artificial
  DNA primer sequence
gaaaaatgaatgagcaagcaaa

SEQ ID NO: 2 - DNA, 20 bp, Artificial
  DNA primer sequence
atggcctcctgcttatgtca

SEQ ID NO: 3 - DNA, 20 bp, Artificial
  DNA primer sequence
gtttccatggcctgtggtat

SEQ ID NO: 4 - DNA, 19 bp, Artificial
  DNA primer sequence
caaaatccttcccgctgag

SEQ ID NO: 5 - DNA, 20 bp, Artificial
  DNA primer sequence
ggaggaagggagaaatgagg

SEQ ID NO: 6 - DNA, 20 bp, Artificial
  DNA primer sequence
cacctccaaaagcttccttg

SEQ ID NO: 7 - DNA, 21 bp, Artificial
  DNA primer sequence
tgcaatttgaccattgaagaa

SEQ ID NO: 8 - DNA, 20 bp, Artificial
  DNA primer sequence
tcaaattggaatgctggaaa

SEQ ID NO: 9 - DNA, 20 bp, Artificial
  DNA primer sequence
tcccaaatgaaggatgaagg

SEQ ID NO: 10 - DNA, 20 bp, Artificial
  DNA primer sequence
gctcctgctgagcttcaact

SEQ ID NO: 11 - DNA, 20 bp, Artificial
  DNA primer sequence
cggacttgacaacctccaag

SEQ ID NO: 12 - DNA, 20 bp, Artificial
  DNA primer sequence
aaagcattctgcacccatgt

SEQ ID NO: 13 - DNA, 20 bp, Artificial
  DNA primer sequence
cctgcttgcagttgactgtg

SEQ ID NO: 14 - DNA, 19 bp, Artificial
  DNA primer sequence
gtgctgggcactgtactgg

SEQ ID NO: 15 - DNA, 21 bp, Artificial
  DNA primer sequence
tggctaaaatgtttggagcac

SEQ ID NO: 16 - DNA, 20 bp, Artificial
  DNA primer sequence
cctgtgagttcttgcccaat

SEQ ID NO: 17 - DNA, 21 bp, Artificial
  DNA primer sequence
cgcattttaaagcaatgatcc

SEQ ID NO: 18 - DNA, 20 bp, Artificial
  DNA primer sequence
tggttgaagagagcagagca

SEQ ID NO: 19 - DNA, 20 bp, Artificial
  DNA primer sequence
aggcagcaaatgggaatttt

SEQ ID NO: 20 - DNA, 20 bp, Artificial
  DNA primer sequence
gagtgacatcacagggcaga

SEQ ID NO: 21 - DNA, 22 bp, Artificial
  DNA primer sequence
tgaagttcttgatcttggcact

SEQ ID NO: 22 - DNA, 22 bp, Artificial
  DNA primer sequence
tttttgaatctgcaactccttg

SEQ ID NO: 23 - DNA, 20 bp, Artificial
  DNA primer sequence
accaattgcttccgtgtcat

SEQ ID NO: 24 - DNA, 22 bp, Artificial
  DNA primer sequence
ctttgcagtgcagataaaaaca

SEQ ID NO: 25 - DNA, 20 bp, Artificial
  DNA primer sequence
attttccagcctcacgaatg

SEQ ID NO: 26 - DNA, 20 bp, Artificial
  DNA primer sequence
tctgggtcagaaaccccata

SEQ ID NO: 27 - DNA, 20 bp, Artificial
  DNA primer sequence
gtgtctcagaggctgcaatg

SEQ ID NO: 28 - DNA, 20 bp, Artificial
  DNA primer sequence
gatgaagcaagctgccttgt

SEQ ID NO: 29 - DNA, 20 bp, Artificial
  DNA primer sequence
aaacggtctcatccacgttc

SEQ ID NO: 30 - DNA, 20 bp, Artificial
  DNA primer sequence
ccaagtgcttgaggttcaca

SEQ ID NO: 31 - DNA, 20 bp, Artificial
  DNA primer sequence
cattgtgttccccacaaaca

SEQ ID NO: 32 - DNA, 20 bp, Artificial
  DNA primer sequence
cgaacacatcgctgacaact

SEQ ID NO: 33 - DNA, 20 bp, Artificial
  DNA primer sequence
gggtcagctggacttcagag

SEQ ID NO: 34 - DNA, 20 bp, Artificial
  DNA primer sequence
aaaatccaaatgcaggaacg

SEQ ID NO: 35 - DNA, 20 bp, Artificial
  DNA primer sequence
agggctggcctgattcttat

SEQ ID NO: 36 - DNA, 22 bp, Artificial
  DNA primer sequence
gaaggcaaatctgcataccttc

SEQ ID NO: 37 - DNA, 21 bp, Artificial
  DNA primer sequence
gaaaaatgaatgagcaagcaa

SEQ ID NO: 38 - DNA, 20 bp, Artificial
  DNA primer sequence
gtcttgccagaggttcatca

SEQ ID NO: 39 - DNA, 20 bp, Artificial
  DNA primer sequence
gtttccatggcctgtggtat

SEQ ID NO: 40 - DNA, 20 bp, Artificial
  DNA primer sequence
ttctccacccagtaggcatc

SEQ ID NO: 41 - DNA, 20 bp, Artificial
  DNA primer sequence
ctggagcccattgagaaaaa

SEQ ID NO: 42 - DNA, 19 bp, Artificial
  DNA primer sequence
caaaatccttcccgctgag

SEQ ID NO: 43 - DNA, 21 bp, Artificial
  DNA primer sequence
tttgcgaactttgacaaatga

SEQ ID NO: 44 - DNA, 20 bp, Artificial
  DNA primer sequence
tcattaaggcccaggtcaag

SEQ ID NO: 45 - DNA, 20 bp, Artificial
  DNA primer sequence
ggaggaagggagaaatgagg

SEQ ID NO: 46 - DNA, 20 bp, Artificial
  DNA primer sequence
cacctccaaaagcttccttg

SEQ ID NO: 47 - DNA, 21 bp, Artificial
  DNA primer sequence
tgcaatttgaccattgaagaa

SEQ ID NO: 48 - DNA, 23 bp, Artificial
  DNA primer sequence
tggaagtgaaagtaagccttttg

SEQ ID NO: 49 - DNA, 20 bp, Artificial
  DNA primer sequence
caacaaaggtgggaatgctt

SEQ ID NO: 50 - DNA, 20 bp, Artificial
  DNA primer sequence
tcaaattggaatgctggaaa

SEQ ID NO: 51 - DNA, 20 bp, Artificial
  DNA primer sequence
tcccaaatgaaggatgaagg

SEQ ID NO: 52 - DNA, 20 bp, Artificial
  DNA primer sequence
gctcctgctgagcttcaact

SEQ ID NO: 53 - DNA, 20 bp, Artificial
  DNA primer sequence
cggacttgacaacctccaag

SEQ ID NO: 54 - DNA, 20 bp, Artificial
  DNA primer sequence
aagtggatgaggttcgctgt

SEQ ID NO: 55 - DNA, 21 bp, Artificial
  DNA primer sequence
gctcctagctcctaatcctga

SEQ ID NO: 56 - DNA, 20 bp, Artificial
  DNA primer sequence
agaagagggaaaccccagag

SEQ ID NO: 57 - DNA, 20 bp, Artificial
  DNA primer sequence
cactatagctgggcctcctg

SEQ ID NO: 58 - DNA, 21 bp, Artificial
  DNA primer sequence
tcctcttcatcacaaccttcc

SEQ ID NO: 59 - DNA, 20 bp, Artificial
  DNA primer sequence
cggggcttctgttttatctg

SEQ ID NO: 60 - DNA, 20 bp, Artificial
  DNA primer sequence
aaagcattctgcacccatgt

SEQ ID NO: 61 - DNA, 20 bp, Artificial
  DNA primer sequence
acccagaacgttttcacaga

SEQ ID NO: 62 - DNA, 20 bp, Artificial
  DNA primer sequence
ccacatccaggaaggtcagt

SEQ ID NO: 63 - DNA, 20 bp, Artificial
  DNA primer sequence
cgacattgaaagcattgaca

SEQ ID NO: 64 - DNA, 23 bp, Artificial
  DNA primer sequence
ccttcgtcatgagacctactttg

SEQ ID NO: 65 - DNA, 20 bp, Artificial
  DNA primer sequence
gaatgcaaaaacccttcacc

SEQ ID NO: 66 - DNA, 20 bp, Artificial
  DNA primer sequence
caggcatttccaagtcgttt

SEQ ID NO: 67 - DNA, 21 bp, Artificial
  DNA primer sequence
cgcattttaaagcaatgatcc

SEQ ID NO: 68 - DNA, 20 bp, Artificial
  DNA primer sequence
tggttgaagagagcagagca

SEQ ID NO: 69 - DNA, 20 bp, Artificial
  DNA primer sequence
aggcagcaaatgggaatttt

SEQ ID NO: 70 - DNA, 20 bp, Artificial
  DNA primer sequence
gagtgacatcacagggcaga

SEQ ID NO: 71 - DNA, 22 bp, Artificial
  DNA primer sequence
tgaagttcttgatcttggcact

SEQ ID NO: 72 - DNA, 22 bp, Artificial
  DNA primer sequence
tttttgaatctgcaactccttg

SEQ ID NO: 73 - DNA, 20 bp, Artificial
  DNA primer sequence
accaattgcttccgtgtcat

SEQ ID NO: 74 - DNA, 22 bp, Artificial
  DNA primer sequence
ctttgcagtgcagataaaaaca

SEQ ID NO: 75 - DNA, 20 bp, Artificial
  DNA primer sequence
attttccagcctcacgaatg

SEQ ID NO: 76 - DNA, 20 bp, Artificial
  DNA primer sequence
accggtaggtatgggtctcc

SEQ ID NO: 77 - DNA, 23 bp, Artificial
  DNA primer sequence
cacaagttcccttcttttcatgt

SEQ ID NO: 78 - DNA, 20 bp, Artificial
  DNA primer sequence
tctgggtcagaaaccccata

SEQ ID NO: 79 - DNA, 20 bp, Artificial
  DNA primer sequence
gtgtctcagaggctgcaatg

SEQ ID NO: 80 - DNA, 20 bp, Artificial
  DNA primer sequence
gatgaagcaagctgccttgt

SEQ ID NO: 81 - DNA, 20 bp, Artificial
  DNA primer sequence
gccatccagccttcttacaa

SEQ ID NO: 82 - DNA, 19 bp, Artificial
  DNA primer sequence
gtgctgggcactgtactgg

SEQ ID NO: 83 - DNA, 20 bp, Artificial
  DNA primer sequence
cctgcttgcagttgactgtg

SEQ ID NO: 84 - DNA, 20 bp, Artificial
  DNA primer sequence
ccctgttgagagggtgacat

SEQ ID NO: 85 - DNA, 20 bp, Artificial
  DNA primer sequence
gcccaaggataggcgtaaat

SEQ ID NO: 86 - DNA, 20 bp, Artificial
  DNA primer sequence
ccaacttcccaaggactgtg

SEQ ID NO: 87 - DNA, 20 bp, Artificial
  DNA primer sequence
tgcttgcccaaaagtattca

SEQ ID NO: 88 - DNA, 20 bp, Artificial
  DNA primer sequence
ggcagctctgaacaaaatcc

SEQ ID NO: 89 - DNA, 20 bp, Artificial
  DNA primer sequence
aatgcacaaatgccacacat

SEQ ID NO: 90 - DNA, 20 bp, Artificial
  DNA primer sequence
tccaagtgttccaagggtgt

SEQ ID NO: 91 - DNA, 20 bp, Artificial
  DNA primer sequence
tttgcgtgatggaatcaaga

SEQ ID NO: 92 - DNA, 20 bp, Artificial
  DNA primer sequence
ggaaaaggtcgtcccagagt

SEQ ID NO: 93 - DNA, 20 bp, Artificial
  DNA primer sequence
ggaaggaatgccgtgtagaa

SEQ ID NO: 94 - DNA, 20 bp, Artificial
  DNA primer sequence
atctggcccacagcaattta

SEQ ID NO: 95 - DNA, 20 bp, Artificial
  DNA primer sequence
gagccagagctgatgagagc

SEQ ID NO: 96 - DNA, 20 bp, Artificial
  DNA primer sequence
tatcaatggcaaccacatgc

SEQ ID NO: 97 - DNA, 20 bp, Artificial
  DNA primer sequence
cccaaggcagctcaattact

SEQ ID NO: 98 - DNA, 20 bp, Artificial
  DNA primer sequence
ggaaggggaatcactggttt

SEQ ID NO: 99 - DNA, 20 bp, Artificial
  DNA primer sequence
ctcggaaaatccctgctttc

SEQ ID NO: 100 - DNA, 20 bp, Artificial
  DNA primer sequence
ggccatgtagctcaggatgt

SEQ ID NO: 101 - DNA, 20 bp, Artificial
  DNA primer sequence
tgcacgaatgaaagatgctc

SEQ ID NO: 102 - DNA, 20 bp, Artificial
  DNA primer sequence
ttgttttgccattccagtca

SEQ ID NO: 103 - DNA, 20 bp, Artificial
  DNA primer sequence
tagacgcaggggtcaaattc

SEQ ID NO: 104 - DNA, 20 bp, Artificial
  DNA primer sequence
ctcaggtccaagtccgagtg

SEQ ID NO: 105 - DNA, 21 bp, Artificial
  DNA primer sequence
cattcaccatgtgccacaata

SEQ ID NO: 106 - DNA, 21 bp, Artificial
  DNA primer sequence
ccatggcagtcaattctatga

SEQ ID NO: 107 - DNA, 20 bp, Artificial
  DNA primer sequence
cttgccagatggctgtcata

SEQ ID NO: 108 - DNA, 20 bp, Artificial
  DNA primer sequence
gcttccttcatccctccttt

SEQ ID NO: 109 - DNA, 20 bp, Artificial
  DNA primer sequence
gctgccacatgcaagaagta

SEQ ID NO: 110 - DNA, 18 bp, Artificial
  DNA primer sequence
cagcacagtgtccgcatc

SEQ ID NO: 111 - DNA, 20 bp, Artificial
  DNA primer sequence
agatcacagcagccttcctg

SEQ ID NO: 112 - DNA, 20 bp, Artificial
  DNA primer sequence
ggatggagtggaagtgcttg

SEQ ID NO: 113 - DNA, 20 bp, Artificial
  DNA primer sequence
ggtctttccctgctctgaca

SEQ ID NO: 114 - DNA, 20 bp, Artificial
  DNA primer sequence
gtccacacaaccgctcctat

SEQ ID NO: 115 - DNA, 20 bp, Artificial
  DNA primer sequence
gccattgacttcttcccaag

SEQ ID NO: 116 - DNA, 20 bp, Artificial
  DNA primer sequence
gggaaagctgcttcctgaat

SEQ ID NO: 117 - DNA, 49 bp, Artificial
  DNA primer sequence
ctacaaacaaacaaacattatcaatagtaaaataaatccagagaaatca

SEQ ID NO: 118 - DNA, 49 bp, Artificial
  DNA primer sequence
ataccaataatccaattcatatcatagtaaaataaatccagagaaatcg

SEQ ID NO: 119 - DNA, 41 bp, Artificial
  DNA primer sequence
tcaaaatctcaaatactcaaatcacaggccaaaaggaagca

SEQ ID NO: 120 - DNA, 40 bp, Artificial
  DNA primer sequence
ttactacacaatatactcatcaataggccaaaaggaagcc

SEQ ID NO: 121 - DNA, 46 bp, Artificial
  DNA primer sequence
ttacctttatacctttctttttacgtcttggtgttcattatcttct

SEQ ID NO: 122 - DNA, 46 bp, Artificial
  DNA primer sequence
ttacttcactttctatttacaatcgtcttggtgttcattatcttcc

SEQ ID NO: 123 - DNA, 44 bp, Artificial
  DNA primer sequence
tcatttacctttaatccaataatccagctgtatagcagagttcg

SEQ ID NO: 124 - DNA, 44 bp, Artificial
  DNA primer sequence
caattcatttaccaatttaccaatcagctgtatagcagagttcc

SEQ ID NO: 125 - DNA, 48 bp, Artificial
  DNA primer sequence
tcaattaccttttcaatacaatacatacttagactactacctcgatga

SEQ ID NO: 126 - DNA, 45 bp, Artificial
  DNA primer sequence
cttttcaattacttcaaatcttcacttagactactacctcgatgg

SEQ ID NO: 127 - DNA, 45 bp, Artificial
  DNA primer sequence
aatcaatcttcattcaaatcatcatcaaagtgattttgggacaac

SEQ ID NO: 128 - DNA, 46 bp, Artificial
  DNA primer sequence
caatatcatcatctttatcattacctcaaagtgattttgggacaat

SEQ ID NO: 129 - DNA, 44 bp, Artificial
  DNA primer sequence
ttactcaaaatctacactttttcacttgtcaatagtcaagggga

SEQ ID NO: 130 - DNA, 43 bp, Artificial
  DNA primer sequence
ttcaatcattcaaatctcaactttttgtcaatagtcaaggggg

SEQ ID NO: 131 - DNA, 45 bp, Artificial
  DNA primer sequence
cttttacaatacttcaatacaatcaattacagaccttggatctcc

SEQ ID NO: 132 - DNA, 48 bp, Artificial
  DNA primer sequence
tcatttactcaacaattacaaatcaaaaattacagaccttggatctct

SEQ ID NO: 133 - DNA, 49 bp, Artificial
  DNA primer sequence
aatcctttctttaatctcaaatcaaatgtgaacttagcacttttatcaa

SEQ ID NO: 134 - DNA, 49 bp, Artificial
  DNA primer sequence
aatcctttttactcaattcaatcaaatgtgaacttagcacttttatcag

SEQ ID NO: 135 - DNA, 40 bp, Artificial
  DNA primer sequence
tcatcaatctttcaatttacttacgtgtaccctgactcgc

SEQ ID NO: 136 - DNA, 41 bp, Artificial
  DNA primer sequence
tcaattacttcactttaatccttttgtgtaccctgactcgt

SEQ ID NO: 137 - DNA, 49 bp, Artificial
  DNA primer sequence
ctaactaacaataatctaactaaccagaacctgatatgtacaaatatga

SEQ ID NO: 138 - DNA, 49 bp, Artificial
  DNA primer sequence
atactaactcaactaactttaaaccagaacctgatatgtacaaatatgg

SEQ ID NO: 139 - DNA, 45 bp, Artificial
  DNA primer sequence
ctacatattcaaattactacttacagataaaagatcactgccctt

SEQ ID NO: 140 - DNA, 45 bp, Artificial
  DNA primer sequence
tacatacactaataacatactcatagataaaagatcactgccctc

SEQ ID NO: 141 - DNA, 42 bp, Artificial
  DNA primer sequence
ctttatcaatacatactacaatcagagacttgggggagtttt

SEQ ID NO: 142 - DNA, 41 bp, Artificial
  DNA primer sequence
tcaatcattacacttttcaacaatagacttgggggagtttc

SEQ ID NO: 143 - DNA, 49 bp, Artificial
  DNA primer sequence
tacactttctttctttctttctttagattttattcttggttaggttgag

SEQ ID NO: 144 - DNA, 49 bp, Artificial
  DNA primer sequence
caatttactcatatacatcactttagattttattcttggttaggttgaa

SEQ ID NO: 145 - DNA, 49 bp, Artificial
  DNA primer sequence
cttttcatcaataatcttacctttgtgctgtctttgaaatgtaaacttt

SEQ ID NO: 146 - DNA, 48 bp, Artificial
  DNA primer sequence
cttctcattaacttacttcataattgctgtctttgaaatgtaaacttg

SEQ ID NO: 147 - DNA, 42 bp, Artificial
  DNA primer sequence
aatctaacaaactcatctaaatacgtggacactgaagagaca

SEQ ID NO: 148 - DNA, 42 bp, Artificial
  DNA primer sequence
caattaactacatacaatacatacgtggacactgaagagact

SEQ ID NO: 149 - DNA, 48 bp, Artificial
  DNA primer sequence
aatcatactcaactaatcattcaacataactttctacagaagttctgg

SEQ ID NO: 150 - DNA, 49 bp, Artificial
  DNA primer sequence
caatataccaatatcatcatttactcataactttctacagaagttctga

SEQ ID NO: 151 - DNA, 38 bp, Artificial
  DNA primer sequence
tcattcatatacataccaattcataagcaggcccaagg

SEQ ID NO: 152 - DNA, 38 bp, Artificial
  DNA primer sequence
taatcttctatatcaacatcttacaagcaggcccaagc

SEQ ID NO: 153 - DNA, 49 bp, Artificial
  DNA primer sequence
ctaaatacttcacaattcatctaaaacactcattgagcttatactacac

SEQ ID NO: 154 - DNA, 49 bp, Artificial
  DNA primer sequence
ctttctacattattcacaacattaaacactcattgagcttatactacat

SEQ ID NO: 155 - DNA, 46 bp, Artificial
  DNA primer sequence
aaacaaacttcacatctcaataatacttctgtaaaacacacgctac

SEQ ID NO: 156 - DNA, 46 bp, Artificial
  DNA primer sequence
tcaatcataatctcataatccaatacttctgtaaaacacacgctag

SEQ ID NO: 157 - DNA, 49 bp, Artificial
  DNA primer sequence
aaactaacatcaatacttacatcaatgaaaaattagaacaacagaaaca

SEQ ID NO: 158 - DNA, 49 bp, Artificial
  DNA primer sequence
tatactatcaactcaacaacatatatgaaaaattagaacaacagaaacg

SEQ ID NO: 159 - DNA, 42 bp, Artificial
  DNA primer sequence
attattcacttcaaactaatctacggattcaattcctcctgg

SEQ ID NO: 160 - DNA, 42 bp, Artificial
  DNA primer sequence
tatatacacttctcaataactaacggattcaattcctcctgc

SEQ ID NO: 161 - DNA, 49 bp, Artificial
  DNA primer sequence
tacactttaaacttactacactaaacactgatatcaagatactggattg

SEQ ID NO: 162 - DNA, 49 bp, Artificial
  DNA primer sequence
ctacttcatatactttatactacaacactgatatcaagatactggattc

SEQ ID NO: 163 - DNA, 49 bp, Artificial
  DNA primer sequence
caattcaaatcacaataatcaatcttcaaacaaatccaaagttatcaaa

SEQ ID NO: 164 - DNA, 49 bp, Artificial
  DNA primer sequence
aatctacaaatccaataatctcatttcaaacaaatccaaagttatcaag

SEQ ID NO: 165 - DNA, 45 bp, Artificial
  DNA primer sequence
ctttctatctttctactcaataatcttgaaggccttgtctataca

SEQ ID NO: 166 - DNA, 45 bp, Artificial
  DNA primer sequence
tcaactaactaatcatctatcaatcttgaaggccttgtctatact

SEQ ID NO: 167 - DNA, 41 bp, Artificial
  DNA primer sequence
tcaacaatcttttacaatcaaatcgggcagatccaagtagc

SEQ ID NO: 168 - DNA, 42 bp, Artificial
  DNA primer sequence
tcatttcaatcaatcatcaacaatagggcagatccaagtaga

SEQ ID NO: 169 - DNA, 49 bp, Artificial; identical to SEQ ID NO: 117
  DNA primer sequence

SEQ ID NO: 170 - DNA, 49 bp, Artificial; identical to SEQ ID NO: 118
  DNA primer sequence

SEQ ID NO: 171 - DNA, 41 bp, Artificial; identical to SEQ ID NO: 119
  DNA primer sequence

SEQ ID NO: 172 - DNA, 40 bp, Artificial; identical to SEQ ID NO: 120
  DNA primer sequence

SEQ ID NO: 173 - DNA, 46 bp, Artificial; identical to SEQ ID NO: 121
  DNA primer sequence

SEQ ID NO: 174 - DNA, 46 bp, Artificial; identical to SEQ ID NO: 122
  DNA primer sequence

SEQ ID NO: 175 - DNA, 49 bp, Artificial; identical to SEQ ID NO: 143
  DNA primer sequence

SEQ ID NO: 176 - DNA, 49 bp, Artificial; identical to SEQ ID NO: 144
  DNA primer sequence

SEQ ID NO: 177 - DNA, 44 bp, Artificial; identical to SEQ ID NO: 124
  DNA primer sequence

SEQ ID NO: 178 - DNA, 44 bp, Artificial; identical to SEQ ID NO: 123
  DNA primer sequence

SEQ ID NO: 179 - DNA, 48 bp, Artificial; identical to SEQ ID NO: 125
  DNA primer sequence

SEQ ID NO: 180 - DNA, 45 bp, Artificial; identical to SEQ ID NO: 126
  DNA primer sequence

SEQ ID NO: 181 - DNA, 45 bp, Artificial; identical to SEQ ID NO: 127
  DNA primer sequence

SEQ ID NO: 182 - DNA, 46 bp, Artificial; identical to SEQ ID NO: 128
  DNA primer sequence

SEQ ID NO: 183 - DNA, 44 bp, Artificial; identical to SEQ ID NO: 129
  DNA primer sequence

SEQ ID NO: 184 - DNA, 43 bp, Artificial; identical to SEQ ID NO: 130
  DNA primer sequence

SEQ ID NO: 185 - DNA, 45 bp, Artificial; identical to SEQ ID NO: 131
  DNA primer sequence

SEQ ID NO: 186 - DNA, 48 bp, Artificial; identical to SEQ ID NO: 132
  DNA primer sequence

SEQ ID NO: 187 - DNA, 49 bp, Artificial; identical to SEQ ID NO: 133
  DNA primer sequence

SEQ ID NO: 188 - DNA, 49 bp, Artificial; identical to SEQ ID NO: 134
  DNA primer sequence

SEQ ID NO: 189 - DNA, 40 bp, Artificial; identical to SEQ ID NO: 135
  DNA primer sequence

SEQ ID NO: 190 - DNA, 41 bp, Artificial; identical to SEQ ID NO: 136
  DNA primer sequence

SEQ ID NO: 191 - DNA, 49 bp, Artificial; identical to SEQ ID NO: 137
  DNA primer sequence

SEQ ID NO: 192 - DNA, 49 bp, Artificial; identical to SEQ ID NO: 138
  DNA primer sequence

SEQ ID NO: 193 - DNA, 48 bp, Artificial
  DNA primer sequence
tacatcaacaattcattcaatacaaaatttacaccactatactctcag

SEQ ID NO: 194 - DNA, 48 bp, Artificial
  DNA primer sequence
tcaatcaattacttactcaaatacaaatttacaccactatactctcaa

SEQ ID NO: 195 - DNA, 49 bp, Artificial
  DNA primer sequence
ctttttcaatcactttcaattcataccatttcagatacaccttttatac

SEQ ID NO: 196 - DNA, 49 bp, Artificial
  DNA primer sequence
aatccttttacattcattacttacaccatttcagatacaccttttatat

SEQ ID NO: 197 - DNA, 46 bp, Artificial
  DNA primer sequence
ctataaacatattacattcacatcgggtaaaattcagtaaggttgg

SEQ ID NO: 198 - DNA, 47 bp, Artificial
  DNA primer sequence
ctttctattcatctaaatacaaacagggtaaaattcagtaaggttga

SEQ ID NO: 199 - DNA, 48 bp, Artificial; identical to SEQ ID NO: 146
  DNA primer sequence

SEQ ID NO: 200 - DNA, 49 bp, Artificial; identical to SEQ ID NO: 145
  DNA primer sequence

SEQ ID NO: 201 - DNA, 42 bp, Artificial; identical to SEQ ID NO: 147
  DNA primer sequence

SEQ ID NO: 202 - DNA, 42 bp, Artificial; identical to SEQ ID NO: 148
  DNA primer sequence

SEQ ID NO: 203 - DNA, 48 bp, Artificial; identical to SEQ ID NO: 149
  DNA primer sequence

SEQ ID NO: 204 - DNA, 49 bp, Artificial; identical to SEQ ID NO: 150
  DNA primer sequence

SEQ ID NO: 205 - DNA, 38 bp, Artificial; identical to SEQ ID NO: 151
  DNA primer sequence

SEQ ID NO: 206 - DNA, 38 bp, Artificial; identical to SEQ ID NO: 152
  DNA primer sequence

SEQ ID NO: 207 - DNA, 49 bp, Artificial; identical to SEQ ID NO: 153
  DNA primer sequence

SEQ ID NO: 208 - DNA, 49 bp, Artificial; identical to SEQ ID NO: 154
  DNA primer sequence

SEQ ID NO: 209 - DNA, 46 bp, Artificial; identical to SEQ ID NO: 155
  DNA primer sequence

SEQ ID NO: 210 - DNA, 46 bp, Artificial; identical to SEQ ID NO: 156
  DNA primer sequence

SEQ ID NO: 211 - DNA, 49 bp, Artificial; identical to SEQ ID NO: 157
  DNA primer sequence

SEQ ID NO: 212 - DNA, 49 bp, Artificial; identical to SEQ ID NO: 158
  DNA primer sequence

SEQ ID NO: 213 - DNA, 42 bp, Artificial; identical to SEQ ID NO: 160
  DNA primer sequence

SEQ ID NO: 214 - DNA, 42 bp, Artificial; identical to SEQ ID NO: 159
  DNA primer sequence

SEQ ID NO: 215 - DNA, 45 bp, Artificial; identical to SEQ ID NO: 139
  DNA primer sequence

SEQ ID NO: 216 - DNA, 45 bp, Artificial; identical to SEQ ID NO: 140
  DNA primer sequence

SEQ ID NO: 217 - DNA, 42 bp, Artificial; identical to SEQ ID NO: 141
  DNA primer sequence

SEQ ID NO: 218 - DNA, 41 bp, Artificial; identical to SEQ ID NO: 142
  DNA primer sequence

SEQ ID NO: 219 - DNA, 47 bp, Artificial
  DNA primer sequence
ctaattactaacatcactaacaatacagagttaaatgaagagtctca

SEQ ID NO: 220 - DNA, 47 bp, Artificial
  DNA primer sequence
atcattacaatccaatcaattcatacagagttaaatgaagagtctcg

SEQ ID NO: 221 - DNA, 49 bp, Artificial
  DNA primer sequence
aatcttactacaaatcctttcttttggtggctaatacattaacattaat

SEQ ID NO: 222 - DNA, 49 bp, Artificial
  DNA primer sequence
ttcataactacaatacatcatcattggtggctaatacattaacattaac

SEQ ID NO: 223 - DNA, 48 bp, Artificial
  DNA primer sequence
tacacaatcttttcattacatcatagatcagttagaaattaaatgcag

SEQ ID NO: 224 - DNA, 48 bp, Artificial
  DNA primer sequence
cttttcaaatcaatactcaactttgatggccttacgagaactaaatat

SEQ ID NO: 225 - DNA, 48 bp, Artificial
  DNA primer sequence
aatctacactaacaatttcataaccagacgagttgtttaaaagaacta

SEQ ID NO: 226 - DNA, 47 bp, Artificial
  DNA primer sequence
atacaatctaacttcactattacaagacgagttgtttaaaagaactc

SEQ ID NO: 227 - DNA, 49 bp, Artificial
  DNA primer sequence
tacaaatcatcaatcactttaatcatgcaacgaaatcttagtttagaaa

SEQ ID NO: 228 - DNA, 49 bp, Artificial
  DNA primer sequence
tcaatcatctttatacttcacaatatgcaacgaaatcttagtttagaac

SEQ ID NO: 229 - DNA, 54 bp, Artificial
  DNA primer sequence
atactacatcataatcaaacatcagggaactttacattgtaagagaaaacaaaa

SEQ ID NO: 230 - DNA, 54 bp, Artificial
  DNA primer sequence
ataccaataatccaattcatatcagggaactttacattgtaagagaaaacaaag

SEQ ID NO: 231 - DNA, 52 bp, Artificial
  DNA primer sequence
tcataatctcaacaatctttctttttctctcggaaatcattaactgtctcat

SEQ ID NO: 232 - DNA, 50 bp, Artificial
  DNA primer sequence
ctatctatctaactatctatatcactctcggaaatcattaactgtctcac

SEQ ID NO: 233 - DNA, 54 bp, Artificial
  DNA primer sequence
aatcttactacaaatcctttcttttattgtttttcaacttctgcatcaaataat

SEQ ID NO: 234 - DNA, 54 bp, Artificial
  DNA primer sequence
tcatcaatcaatctttttcacttttattgtttttcaacttctgcatcaaataac

SEQ ID NO: 235 - DNA, 41 bp, Artificial
  DNA primer sequence
aatctaacaaactcatctaaatacgaccaccgagcagcgac

SEQ ID NO: 236 - DNA, 43 bp, Artificial
  DNA primer sequence
caattaactacatacaatacatacatgaccaccgagcagcgat

SEQ ID NO: 237 - DNA, 51 bp, Artificial
  DNA primer sequence
tcaattacttcactttaatcctttctctgagtaagatcttgctcaatctct

SEQ ID NO: 238 - DNA, 51 bp, Artificial
  DNA primer sequence
ttcacttttcaatcaactttaatcctctgagtaagatcttgctcaatctca

SEQ ID NO: 239 - DNA, 44 bp, Artificial
  DNA primer sequence
caattcaaatcacaataatcaatcaggaaggcaaacacctcctt

SEQ ID NO: 240 - DNA, 44 bp, Artificial
  DNA primer sequence
aatctacactaacaatttcataacaggaaggcaaacacctcctc

SEQ ID NO: 241 - DNA, 53 bp, Artificial
  DNA primer sequence
tacatacactaataacatactcatagattcagaaataaattggaaattgaaga

SEQ ID NO: 242 - DNA, 53 bp, Artificial
  DNA primer sequence
ctacaaacaaacaaacattatcaaagattcagaaataaattggaaattgaagt

SEQ ID NO: 243 - DNA, 45 bp, Artificial
  DNA primer sequence
aaactaacatcaatacttacatcatggcggcgattacagaaaaca

SEQ ID NO: 244 - DNA, 44 bp, Artificial
  DNA primer sequence
atcatacatacatacaaatctacaggcggcgattacagaaaacg

SEQ ID NO: 245 - DNA, 42 bp, Artificial
  DNA primer sequence
ctttaatctcaatcaatacaaatcatgtgggcatggctggac

SEQ ID NO: 246 - DNA, 43 bp, Artificial
  DNA primer sequence
ttacctttatacctttctttttacgatgtgggcatggctggat

SEQ ID NO: 247 - DNA, 39 bp, Artificial
  DNA primer sequence
tcaactaactaatcatctatcaatggcacaggcctggcg

SEQ ID NO: 248 - DNA, 39 bp, Artificial
  DNA primer sequence
atactaactcaactaactttaaacggcacaggcctggca

SEQ ID NO: 249 - DNA, 49 bp, Artificial
  DNA primer sequence
tcatttaccaatctttctttatacctcaccagagttcttctagcatgat

SEQ ID NO: 250 - DNA, 48 bp, Artificial
  DNA primer sequence
caattcatttaccaatttaccaattcaccagagttcttctagcatgac

SEQ ID NO: 251 - DNA, 45 bp, Artificial
  DNA primer sequence
tcaaaatctcaaatactcaaatcacctctcacaaaagacccctta

SEQ ID NO: 252 - DNA, 45 bp, Artificial
  DNA primer sequence
aatctacaaatccaataatctcatcctctcacaaaagaccccttc

(19) United States
(12) Patent Application Publication
(10) Pub. No.: US 2010/0311052 A1
(43) Pub. Date: Dec. 9, 2010

(54) PROGNOSTIC METHOD FOR THE DETERMINATION OF THE SUITABILITY OF BIOPHARMACEUTICAL TREATMENT

(75) Inventors: Klaus Bendtzen, Lynge (DK); Christian Enevold, Kobenhavn 0 (DK)

Correspondence Address: MCDERMOTT WILL & EMERY LLP, 600 13TH STREET, N.W., WASHINGTON, DC, 20005-3096, US

(73) Assignee: BioMonitor A/S, Copenhagen (DK)

(21) Appl. No.: 12/667,096

(22) PCT Filed: Jun. 26, 2008
(86) PCT No.: PCT/EP2008/058163

§ 371 Date: Aug. 24, 2010

(30) Foreign Application Priority Data
Jun. 29, 2007 (DK) PA 2007 00950

(51) Int. Cl. C12Q 1/68

(52) U.S. Cl. 435/6

(57) ABSTRACT

The invention refers to a method for the prognosis of a disease in a subject by the administration of a biopharmaceutical treatment in a subject suffering from, or likely to suffer from the disease, the method involving the analysis of SNP polymorphisms in the subjects pattern recognition receptor genes (PRRs), eg the analysis of polymorphisms' with the purpose of predicting the response of anti-TNFx antibody therapy in rheumatoid arthritis patients. Also the response to Beta-interferon in multiple sclerosis patients may be predicted. The genes whose polymorphisms are analysed may be TLRs, NOD-like receptors or retinoic acid-inducible gene I-like receptors (RLR).

### FIELD OF INVENTION

The present invention relates to methods for determining whether a patient is likely to respond to a medical treatment, such as monoclonal antibody treatment, by the identification of nucleic acid variants which are indicators for the prognosis for treatments with the biopharmaceutical.

### BACKGROUND TO THE INVENTION

According to the Pharmaceutical Research and Manufacturers of America (PhRMA) millions of people have benefited from medicines and vaccines developed through biotechnology, and according to recent reports there are numerous further biopharmaceuticals for the treatment of more than 100 diseases currently in development. In their survey, the PhRMA identified 324 biotechnology medicines in development for nearly 150 diseases. These include 154 medicines for cancer, 43 for infectious diseases, 26 for autoimmune diseases and 17 for AIDS/HIV and related conditions. These potential medicines, all of which are either in human clinical trials or under review by the Food and Drug Administration, will bolster the list of 108 biotechnology medicines already approved and available to patients. The report is available from http://www.phrma.org/new_medicines_in_development_for_biotechnology/, and is hereby incorporated by reference.

Key to this successful development of biopharmaceutical agents has been the creation of humanised or fully human protein agents which are designed to evade recognition by the human immune system as foreign agents.

However, despite the use of humanized or fully human biopharmaceutical agents (drugs), response failure is increasingly being realized in the use of biopharmaceuticals. One possible cause is that treatment responses of diseases related to innate immunity, e.g. chronic immunoinflammatory diseases, depend upon genetic variants of the major pattern recognition receptors (PRR) of the innate immune system, notably the Toll-like receptors (TLR), the NOD-like receptors (NLR), and the retinoic acid-inducible gene I-like receptors (RLR). Another cause of response failure is the development of host antibodies to the drugs, which can greatly decrease the efficacy of the biopharmaceutical drug, or completely obliterate the benefit of taking the drug, resulting in considerable wasted expenditure on ineffective therapy and lost time in the treatment of the disorder which can have catastrophic effects in terms of the development of irreversible tissue damage in the patient. Antibody development (=drug immunogenicity) depends upon a host of factors eventually triggering B-cells to produce anti-drug antibodies, and it has recently become clear that innate immune functions are central in triggering both T-cell-dependent and T-cell-independent antibody production by B-cells.

There is therefore a need for methods to determine the likelihood that an individual will benefit from biopharmaceutical treatments, a process that will save the patient from receiving ineffective, and possibly dangerous, treatments (e.g. in the case of a severe immune response against the biopharmaceutical), ensure early selection of appropriate treatment, and also considerably reduce expenditure on ineffective treatments.

WO 2007/025989 refers to a method of identifying a subject at risk of having an indication associated with altered innate immunity which comprises detecting nucleic acid variants, such as single nucleotide polymorphisms (SNPs) present in a Toll-Like Receptor gene (TLR). Whilst it is hypothesized in WO 2007/025989 that detection of TLR variants may be used to identify a subject at risk of having a modified response to a therapy for a disease; no data was presented in WO 2007/025989 which illustrates this hypothesis and notably the only therapies which were mentioned in this regards were NSAID therapy and vaccination.

The immune system is decisive in preventing infections, and the system is of central pathogenic importance in acute and chronic diseases characterized by inflammation, autoimmunity, tissue destruction and -repair, and ageing [1]. There are two major immune systems, the innate and the adaptive immune systems [2, 3]. The latter has been investigated for decades, also for roles in ageing. In contrast, the functions of the innate immune system are much less known, partly because the essential signal molecules of this system, the Pattern-Recognition Receptors (PRR), have only recently been recognized [4-6]. These receptors are now characterized as “the top of the pyramid” in the human immune system, because PRRs to a great extent govern the functions of both immune systems and therefore are likely to be of importance for many if not all processes influenced by immune cells, including antibody-producing plasma cells and the plasma cell-precursors, B-cells.

### Toll-Like Receptors (TLRs) and Other PRRs

Toll-like receptors (TLRs), NOD-like receptors (NLRs), and retinoic acid-inducible gene I (RIG-I) like receptors (RLRs) constitutes germline-encoded families of molecules essentially involved in innate immunity [3, 7]. Innate immunity is initiated or activated by structures referred to as pathogen-associated molecular patterns (PAMP), which are recognized by corresponding pattern recognition receptors (PRR). The best-characterized PAMPs are microbial peptidoglycans, lipopolysaccharides (LPS), flagellin, zymosan, mannans, bacterial and viral DNA and RNA and bacterial CpG-containing DNA, but ‘endogenous’ components such as heat-shock proteins and fibrinogen, may also be recognized. Dendritic cells (DC), macrophages (MØ) and B- and T-cells express PRR, and TLRs, NLRs, and RLRs constitute important subgroups of PRRs.

TLRs, NLRs, and RLRs are essential for detecting PAMPs, and by doing so execute the first line of defense for pathogen recognition [8, 9]. During these processes, TLRs, NLRs, and RLRs activate cells of the host defense, including but not limited to DC, MØ, B- and T-cells. As these cell types are critically involved not only in host defense but also in the pathogenesis of a vast range of acute and chronic immunoinflammatory diseases, TLRs, NLRs, and RLRs may to some extent govern induction and maintenance of common diseases [10-12]. These include the following and many others: rheumatic diseases (rheumatoid arthritis (RA), ankylosing spondylitis, etc), inflammatory bowel diseases (Crohn's disease, ulcerative colitis), inflammatory skin diseases (psoriasis, eczema, etc), inflammatory diseases of the brain and peripheral nerves (multiple sclerosis, various neuropathies, etc), vascular inflammatory diseases (arteriosclerosis), periodontitis, and inflammatory diseases of muscles (heart and skeletal), eyes, lungs, liver, kidneys, bone and endocrine organs, incl. type I and type 2 diabetes.

TLRs are divided into five subfamilies on the basis of amino acid sequence homology: TLR-1, 2, 6 and 10, TLR-3, TLR-4, TLR-5, and TLR-7, 8 and 9. The extracellular regions of TLRs contain leucine-rich repeats flanked by cysteine-rich motifs. The cytoplasmic regions of TLRs all contain a TOLL/IL-1 receptor (TIR) homology domain which is critical for signaling.

The NOD-like receptors (NLRs) are cytoplasmic proteins that may have a variety of functions in regulation of inflammatory and apoptotic responses. Approximately 20 of these proteins have been found in the mammalian genome and include two major subfamilies called NODs and NALPs, the MHC Class II transactivator (CIITA), and some other molecules (e.g. IPAF and BIRC1). The NLR family is known under several different names, including the CATERPILLER (or CLR) or NOD-LRR family.

RIG-1-like receptors (RLRs) are intracellular RNA helicase proteins that participate in the innate immune responses against viruses. They recognize double-stranded RNA produced during virus replication or from synthetic sources.

Because the specificity of TLRs, NLRs, and RLRs (and other innate immune receptors) cannot easily be changed in the course of evolution, these receptors recognize molecules that are constantly associated with ‘danger’ (i.e. pathogen or cell stress etc.), that are not subject to mutation, and are highly specific to these threats (i.e. cannot be mistaken for self molecules). Pathogen associated molecules that meet this requirement are usually critical to the pathogen's function and cannot be eliminated or changed through mutation; they are said to be evolutionarily conserved. It is therefore highly surprising that, as described herein, TLR polymorphisms are key determinants in how a subject will respond (or not) to biopharmaceutical treatment, particularly protein based pharmaceuticals which are based upon human protein sequences or designed to mimic human proteins (humanized biopharmaceuticals), such as monoclonal antibodies and beta-interferon. By contrast, pathogen vaccines are designed to present established pathogen antigens to the immune system.

The present invention is based upon the surprising observation that detection of TLR polymorphisms can be used as highly effective indicators of the likelihood of response failure to biopharmaceutical agents, particularly protein drugs, such as monoclonal antibodies and interferon drugs such as IFN-beta, and drugs, which typically, as opposed to vaccines, are designed to be similar or even identical to (human) ‘self’ proteins, and thereby evade the immune system.

### SUMMARY OF THE INVENTION

The present invention provides methods for the prognosis of the development of an immune response to a bio-agent in a subject, such as a biopharmaceutical or diagnostic monoclonal antibody, by the identification of one or more polymorphisms (such as SNPs) present in the genetic code of the subject which encodes one or more toll like receptors (TLRs), NOD-like receptors (NLRs), or RIG-I like receptors (RLRs). The method typically comprises steps a)-c) and optionally d), as referred to herein.

The present invention provides methods for determining whether a subject is likely to benefit from the administration of the bio-agent, such as a biopharmaceutical treatment or antibody diagnostic, by the identification of TLR, NLR, or RLR polymorphisms (such as SNPs) present in the genetic code of the subject. The method typically comprises steps a)-c) and optionally d), as referred to herein.

As disclosed herein, TLR, NLR, and RLR polymorphisms can be indicators for the (likely) prognosis of the development of an immune response to the biopharmaceutical/biodiagnostic and therefore the (likely) prognosis of treatments with the biopharmaceutical or diagnostic.

Therefore, the present invention provides for a method for the prognosis of the treatment of a disease in a subject, said treatment comprising the administration of a biopharmaceutical treatment to the subject, said method comprising the steps of:
a) Obtaining a sample comprising the genetic code from the subject;
b) Determining the presence or absence or copy number of at least 1 polymorphism, such as at least one single nucleotide polymorphism (SNP), in the genetic code (which encodes) for one or more TLRs, NLRs, or RLRs or combinations hereof;
c) Comparing the presence or absence or copy number of the at least one polymorphism, such as at least one SNP, identified in step b) with control data obtained from either
   i) At least one subject which has been successfully treated for the disease using the biopharmaceutical (negative control); and/or
   ii) At least one subject which has developed the disease and has a history of failed treatment of said disease (positive control).

Suitably, in a further step d), from the comparison of the data in step c) the likelihood of the success of the treatment of a disease or prevention of the development of a disease in the subject can be determined.

The method of the invention may be used in relation to preventative therapy, therefore the subject may be suffering from, or may be likely to suffer from the disease.

Therefore, the present invention provides for a method for determination of the suitability of using diagnostic antibody constructs specific for a disease epitope, for the in vivo detection of the disease in a subject, said method comprising the steps of:
a) Obtaining a sample comprising the genetic code from the subject;
b) Determining the presence or absence or copy number of at least 1 polymorphism, such as at least one single nucleotide polymorphism (SNP), in the genetic code (which encodes) for one or more TLRs, NLRs, or RLRs or combinations hereof;
c) Comparing the presence or absence or copy number of the at least one polymorphism, such as at least one SNP, identified in step b) with control data obtained from either
   i) At least one subject which has developed an immune response to the biopharmaceutical; and/or (positive control);
   ii) At least one subject which has not developed an immune response to the biopharmaceutical despite repeated administrations of the biopharmaceutical (negative control).

Suitably, from the comparison of the data in step c) the likelihood of the success of the diagnostic antibody constructs in determining the presence (or location) of a disease in the subject can be made, and therefore the suitability of the diagnostic antibody construct for the monitoring of the disease in the patient.

The invention further provides for a method for the identification of one or more polymorphisms of TLR, NLR, or RLR encoding genetic codes or combinations hereof, which are correlated to a prognosis of a subject for the development of an immune response to a bio-agent, such as a biopharmaceutical or diagnostic monoclonal antibody, said method comprising the steps of:
a) Collecting genetic material or information from
   i) a population of subjects which have a history of successful treatment or diagnosis with the bio-agent; and
   ii) a population of subjects which have a history of failed treatment or diagnosis with the bio-agent;
b) For each of the subjects, perform a series of genetic analyses to characterize the polymorphisms present in their PRR, such as TLR, NLR, or RLR genetic material, preferably using a multiplex reaction;
c) Perform statistical analysis of the data obtained in b) to identify polymorphism(s) having a significant correlation to either population i) or population ii).

### BRIEF DESCRIPTION OF THE FIGURES

FIG. 1, A and B: The uppermost graphs show the Median Fluorescence Intensity (MFI)-signals from a plate run, while the lower graphs show the ratios of the allelic MFIs (i.e. C-allelic ratio=C-allelic MFI/(C-allelic MFI+T-allelic MFI)). In FIG. 1A the graphs show the three genotype groups nicely separated with the heterozygous group being almost exactly in the middle, while the two homozygous groups are located in close vicinity of the axes, this is the ideal distribution. In FIG. 1B the graphs graph depicts one of the more difficult SNP-graphs with a skewed distribution of the groups.

### DETAILED DESCRIPTION OF THE INVENTION

The term ‘prognostic’ as used herein refers to an indicator of the likely course of a disease. In the case of the present invention, the prognosis is typically performed based on the likely response of the disease (or future disease) in the subject compared to the response if the treatment was not given. Suitably the prognosis may be positive, i.e. it is likely that the treatment will result in an improved prognosis of the disease (i.e. likely to benefit), possibly even a cure, or negative, i.e. the treatment will not result in an improved prognosis and may even cause excessive undesirable side effects.

The term ‘encodes’ within the context of the present invention is not necessarily limited to the coding sequence (of the TLR, NLR or RLR), but may in one embodiment also include the non-coding regions of the TLR genes, such as promoter elements, introns, 3′ and 5′ untranslated regions, and in one embodiment enhancer elements. In this respect the term ‘the genetic code which encodes one or more TLRs, NLRs, or RLRs is equivalent to the term TLR, NLR, or RLR genes’ and encompasses the coding sequence (of the TLR, NLR, or RLR), and the non-coding regions of the TLR, NLR, or RLR genes, such as promoter elements, introns, 3′ and 5′ untranslated regions, and in one embodiment enhancer elements.

In some embodiments the one or more polymorphisms is present in one or more PRR genes independently selected from the group consisting of the Toll-like receptors (TLR), the NOD-like receptors (NLR), and the retinoic acid-inducible gene I-like receptors (RLR).

The term ‘at least one’ includes ‘one or more’, such as at least two, at least three, at least four, at least five, etc. In one embodiment the term at least one may refer to 1, 2, 3, 4, 5, 6, 7, 8, 9 or 10 (such as in the number of TLRs, NLRs, or RLRs or TLR, NLR, or RLR polymorphisms. In the context of multiplexed reactions the term at least one, may refer to at least 5, such as at least 8, such as at least 10, at least 15, at least 20, at least 25, at least 30. In one embodiment the number of polymorphisms detected, e.g. in a multiplex reaction, may not exceed 40 or may not exceed 50.

The terms ‘biopharmaceutical’ or ‘biopharmaceutical agent” as used herein refers to protein based therapeutic agents, which are produced by means other than direct extraction from a native, non-engineered biological source. The biopharmaceutical according to the invention may be selected from the group consisting of: blood factors, such as Factor VII, Factor VIII and Factor IX, and thrombin, each one in activated or zymogen forms; thrombolytic agents, such as tissue plasminogen activator; hormones, such as insulin, growth hormone, and gonadotropins; haematopoietic growth factors, such as erythropoietin, and colony stimulating factors (GM-CSF, etc.); interferons (interferons-α, -β, -γ, -δ, -ω), cytokine-based products (interleukins, vascular endothelial growth factor (VEGF), etc.); tumour necrosis factors; monoclonal antibodies; and therapeutic enzymes. The biopharmaceutical may suitably be referred to as a protein drug. The biopharmaceuticals of the invention are preferably derived, at least in part, from mammalian/human protein sequences, (e.g. they share at least 80% such as at least 90%, such as at least 95%, such as at least 98% homology or even 100% homology (amino acid sequence identity) with the (equivalent) mammalian/human protein sequence from which they were derived). It is recognized that the biopharmaceuticals may not be 100% identical to the mammalian/human protein sequences from which they are derived—e.g. monoclonal antibodies typically comprise selected or engineered variable/hyper-variable sequences which may not have been directly from the mammalian/human source. In one embodiment, the biopharmaceuticals may also be a fragment of the mammalian/human protein sequence from which is derived (it may comprise, for example as at least 25%, at least 40%, at least 50%, at least 75%, or at least 90% of the mammalian/human protein sequence from which it is derived). In one embodiment, the biopharmaceutical agent may also be a fusion protein comprising protein sequences obtained from two (or more) mammalian/human proteins (or fragments thereof). Biopharmaceuticals may be produced from microbial cells (e.g. recombinant E. coli), mammalian cells, such as mammalian cell lines or transgenic mammals, insect cell culture, and plant cells, such as plant cell cultures or transgenic plants. For production in cell cultures, biopharmaceuticals are typically produced by heterologous expression in expression hosts which are grown in, and/or express the biopharmaceuticals in bioreactors of various configurations.

It is preferable that the term ‘biopharmaceutical’ as used herein does not include vaccines, particularly vaccines derived from pathogenic antigens (such as proteins) or active against pathogenic agents. The term vaccine refers to an antigenic preparation used to establish immunity to a disease. In this respect although the biopharmaceutical agent may cause an immune response in the subject, it is not a vaccine.

In some embodiment the term “biopharmaceutical” or “bio-agent” refers to a endogenous protein compound elicited by another therapeutic drug or medical treatment. In some embodiment the endogenous protein is elicited by a chemotherapy-induced immune response. In some embodiment the endogenous protein is elicited by a radiotherapy-induced immune response. In some embodiments this endogenous protein is high-mobility-group box 1 (HMGB1) alarmin protein.

Accordingly the term “biopharmaceutical treatment” may in some further embodiments encompass the treatment with a non-protein, such as chemotherapy or radiotherapy that elicits an endogenous biopharmaceutical required for the success of the therapy.

Commonly used biopharmaceuticals includes but are not limited to:

Erythropoietin—Treatment of anaemia,
Interferon-α—Treatment of leukaemia
Interferon-β—Treatment of multiple sclerosis
Monoclonal antibody—Treatment of rheumatoid arthritis, multiple sclerosis, Chron's disease.
Colony stimulating factors—Treatment of neutropenia
Glucocerebrosidase—Treatment of Gaucher's disease

The method according to the invention may, therefore, be used for the prognosis of treatment of the above disorders, such as with the above listed biopharmaceuticals.

### Diagnostics

Radio-labeled monoclonal antibodies are routinely used in the monitoring of diseases such as cancers, and some infectious diseases, where it is important to determine the size and/or location of the disease/agent—for example in identifying the presence/location of any secondary metastases. When the development of response failure (either primary or secondary) occurs unnoticed, the patient may be given the ‘all clear’—i.e. a false negative result, this can lead to the cessation of treatment and the latter re-appearance of the disease, often in a far more developed and possibly untreatable condition. Therefore, within the context of the present invention, in one embodiment, the term Bio-agent or biopharmaceutical includes ‘biodiagnostic monoclonal antibody’, such as a radiolabeled biodiagnostic monoclonal antibody.

Therefore in one embodiment, the method of the invention refers to a method for determination of the suitability of using diagnostic antibody constructs in vivo in a subject. Typically the diagnostic antibody constructs are used in the diagnosis or monitoring of a disease, such as cancer, particularly for the continued or repeated use of antibody constructs targeting e.g. cancer antigens to determine effects (efficacy) of repeated anti-cancer treatments. Therefore the present methods can be used to prognostically determine the likelihood of the subject developing host immunity to the diagnostic antibody constructs.

Suitably the subject in the method for determination of the suitability of using diagnostic antibody constructs is either being considered for or is already undergoing, or has already undergone treatment for the disease.

### Monoclonal Antibodies

In a preferred embodiment, the biopharmaceutical according to the invention is a monoclonal antibody.

The term “monoclonal antibody” as used herein typically refers to a single light chain biopharmaceutical which consists of an intact light chain immunoglobulin, or a fragment thereof which comprises at least a variable domain, and at least part of the light chain constant region. The monoclonal antibody is typically free of heavy chain immunoglobulins. Table 1 provides a list of monoclonal antibodies which are suitable biopharmaceuticals according to the invention.

Heavy chain antibodies typically have a molecular weight of approximately 50 kDa, whereas the light chains typically have a molecular weight of approximately 25 kDa. The light and heavy chains are joined together by a disulfide bond near the carboxyl terminus of the light chain. The heavy chain is divided into an Fc portion, which is at the carboxyl terminal (the base of the Y), and a Fab portion, which is at the amino terminal (the arm of the Y). Carbohydrate chains are attached to the Fc portion of the molecule. The Fc portion of the Ig molecule is composed only of heavy chains. The Fc region contains protein sequences common to all Igs as well as determinants unique to the individual classes. These regions are referred to as the constant regions because they do not vary significantly among different Ig molecules within the same class. The Fab portion of the Ig molecule contains both heavy and light chains joined together by a single disulfide bond. One heavy and one light chain pair combine to form the antigen binding site of the antibody. Human light chain antibodies can be of either lambda or kappa isotypes.

The term “intact light chain” refers to a polypeptide which consists of both one or more variable regions and a constant regions (or part thereof) a light chain isotype polypeptide. The intact light chain is the product of the expression of a light chain encoding polynucleotide, taking into account post-translational modifications which may occur during production within the expression system.

### Interferon

Interferon (IFN) is a group of natural proteins produced by many cell types in response to challenge by infectious agents, primarily viruses, but also bacteria and parasites. Natural, partly purified IFN preparations have been used for many years, primarily as therapies against viral infections and certain cancers. From the 1980s recombinant gene technologies allowed mass cultivation and purification from bacterial and mammalian cell cultures. This paved the way for use of IFN in many diseases, including the use of human recombinant IFN-beta in patients with multiple myeloma and multiple sclerosis (MS). Hence, IFN-beta is the first-line treatment of patients with relapsing-remitting MS, as it has been shown to reduce the progression of disability and suppress signs and severity of the disease. However, the development of host antibodies targeting the recombinant IFN greatly reduces the effectiveness of treatment.

Type 1 IFNs, mainly IFN-alpha, have been used as therapy for patients with viral infections, including hepatitis B and C virus, as well as patients with malignant conditions. Composed of a group of at least 23 subtypes of 19-26 kDa (glyco)proteins, IFN-alpha is produced primarily by virus-infected leukocytes but also by many other cell types.

IFN-beta is produced primarily by virus-infected fibroblasts and consists of a group of at least 2 members of 23-42 kDa glycoproteins called IFN-beta1 and IFN-beta3 (IFN-beta2, also known as interleukin-6, does not belong to this group). In contrast to IFN-alpha, IFN-beta is strictly species-specific in that IFN-beta of other species is inactive in human cells. Both IFN-alpha and -beta interfere with replication of many viruses in almost all cell types and, in addition, have antiproliferative and immunomodulatory functions.

There are currently two main therapeutic preparations of recombinant IFN-beta:

IFN-beta-1b is produced by Berlex Laboratories (Montville N.J., USA) and Bayer-Schering (Berlin, Germany) under the trade names Betaseron® and Betaferon® and was the first in use in MS patients. It is produced in E. coli and is therefore non-glycosylated, unlike its natural counterpart. In addition, IFN-beta-1b differs from wild-type IFN-beta in that it lacks the N-terminal amino acid (methionine) and that one amino acid in position 17 is different (cysteine substituted with serine). IFN-beta-1a is produced by Biogen (Cambridge, Mass., USA) under the trade name Avonex® and by Serono Inc. (Rockland, Mass., USA) under the trade name Rebif®. IFN-beta-1a preparations are produced in mammalian Chinese Hamster Ovary cells. The amino acid sequence is identical to native IFN-beta, and it is glycosylated although not exactly equal to the wild-type human IFN-beta.

In one embodiment the biopharmaceutical is beta-interferon, and typically the disease is multiple sclerosis.

A list of interferon-based biopharmaceuticals is provided in table 1.

### Single Nucleotide Polymorphisms

The term ‘Single nucleotide polymorphism’ or ‘SNP’ is a genetic (DNA) sequence variation occurring when a single nucleotide—A, T, C, or G—in the genome (or other shared sequence) differs between members of a species (or between paired chromosomes in an individual). For example, two sequenced DNA fragments from different individuals, AAGCCTA to AAGCTTA, contain a difference in a single nucleotide. In this case there are two alleles: C and T. Almost all common SNPs have only two alleles.

The ‘sample’ is typically a composition which comprises the genomic genetic code of the subject, (i.e. at least the genetic code which comprises genetic code for the one or more PRR, such as the TLR, NLR, or RLR genetic code or a fraction of the PRR, such as TLR, NLR, or RLR genetic code which encompasses the site of the SNP or SNPs). The sample may be in the form of information, e.g. in silico—e.g. the sample may be the genome sequence of the subject. Typically the sample is obtained from the subject in the form of a tissue (e.g. blood) sample, from which the genetic code is obtained or extracted.

The term ‘subject’ as used herein refers to an individual who is either: (i) being considered for treatment, or undergoing treatment, or previously received treatment, wherein the treatment involves the administration of a biopharmaceutical (bio-agent), or (ii) is being considered for diagnosis, or undergoing diagnosis, or has previously undergone diagnosis for a disorder or a disease, wherein the diagnosis involves the administration of a labeled (typically radio-labeled) monoclonal antibody into the body of the subject, wherein the monoclonal antibody (bio-agent) is used to specifically detect and/or localize the presence of the disorder or disease or disease causing agent (see method ‘for determination of the suitability of using diagnostic antibody constructs specific for a disease epitope’ as described herein).

Determining the Presence or Absence or Copy Number of at Least 1 Single Nucleotide Polymorphisms (SNP) in the genetic code which encodes for one or more PRRs, such as TLR, NLR, or RLR.

Single nucleotide polymorphisms may fall within coding sequences of genes, noncoding regions of genes, or in the intergenic regions between genes. SNP's within a coding sequence will not necessarily change the amino acid sequence of the protein that is produced, due to degeneracy of the genetic code. A SNP in which both forms lead to the same polypeptide sequence is termed synonymous (sometimes called a silent mutation)—if a different polypeptide sequence is produced they are termed non-synonymous. SNP's that are not in protein coding regions may still have consequences for gene splicing, transcription factor binding, or the sequence of non-coding RNA. In one embodiment the at least one SNP according to the invention is a SNP present in the coding sequence of the PRR, such as TLR, NLR, or RLR, and preferably introducing an amino acid substitution in the PRR, such as TLR, NLR, or RLR. In one embodiment the at least one SNP according to the invention is present in a non-coding region, such as the untranslated regions (5′UTR and/or 3′UTR), or PRR, such as TLR, NLR, or RLR gene promoter regions (or enhancer elements), or PRR, such as TLR, NLR, or RLR intron sequences, or PRR, such as TLR, NLR, or RLR intron/exon boundaries.

In one embodiment, the characterization of the at least one SNP in step b comprises determining the copy number of the specific SNP—such as determining whether the patient genetic sample is heterozygous or homozygous for the at least one SNP in step b).

Suitably, in one embodiment, the at least one SNP includes at least on SNP within the genetic code which encodes a TLR selected from the group consisting of TLR1, TLR2, TLR3, TLR4, TLR5, TLR6, TLR7, TLR8, TLR9 and TLR 10.

Suitably, in one embodiment, the at least one SNP includes at least on SNP within the genetic code which encodes a TLR selected from the group consisting of TLR2, TLR4, TLR5, and TLR9.

Suitably, in one embodiment, the at least one SNP includes at least on SNP within the genetic code which encodes a TLR selected from the group consisting of TLR3, TLR7 and TLR8.

Suitably, in one embodiment, the at least one SNP includes at least one SNP within the genetic code which encodes a NLR.

Suitably, in one embodiment, the at least one SNP includes at least one SNP within the genetic code which encodes a NLR selected from the group consisting of Nucleotide-binding oligomerization domain protein 1 (NOD1) (also known as CARD4) and Nucleotide-binding oligomerization domain protein 2 (NOD2) (also known as CARD15).

Suitably, in one embodiment, the at least one SNP includes at least one SNP within the genetic code which encodes a RLR.

Suitably, in one embodiment, the at least one SNP includes at least on SNP within the genetic code which encodes a RLR selected from the group consisting of Retinoic acid-inducible gene I (RIG-I), also known as DEAD/H box 58 (DDX58) and Interferon induced with helicase C domain protein 1 (IFIH1), also known as Melanoma differentiation-associated gene 5 (MDA5).

In one embodiment, the at least one SNP may be selected from the group consisting of the SNPs shown in Table 2, table 3 or in table 2 of WO 2007/025989.

In one embodiment, the at least one SNP may be a SNP found in the genetic code which encodes a TLR selected from the group consisting of TLR5, TLR7, TLR8 and TLR 9.

In one embodiment, the at least one of the SNP is an SNP found in the genetic code which encodes a TLR selected from the group consisting of TLR-1, 2, 6 and 10, such as TLR2.2, TLR6.3, TLR9.1, TLR10.4, and TLR10.5, or any combination thereof.

In one embodiment, the at least one of the SNP is an SNP found in the genetic code which encodes a TLR-1.

In one embodiment, the at least one of the SNP is an SNP found in the genetic code which encodes a TLR-2, such as TLR2.2.

In one embodiment, the at least one of the SNP is an SNP found in the genetic code which encodes a TLR-6, such as TLR6.3.

In one embodiment, the at least one of the SNP is an SNP found in the genetic code which encodes a TLR-10, such as TLR10.4, TLR10.5, or any combination hereof.

In one embodiment, the at least one of the SNP is an SNP found in the genetic code which encodes a TLR-4.

In one embodiment, the at least one of the SNP is an SNP found in the genetic code which encodes a TLR-5, such as TLR5.3.

In one embodiment, the at least one of the SNP is an SNP found in the genetic code which encodes a TLR selected from the group consisting of TLR-7, 8 and 9.

In one embodiment, the at least one of the SNP is an SNP found in the genetic code which encodes a TLR-7.

In one embodiment, the at least one of the SNP is an SNP found in the genetic code which encodes a TLR-8, such as TLR8.1.

In one embodiment, the at least one of the SNP is an SNP found in the genetic code which encodes a TLR-9, such as TLR9.1.

In one embodiment, the at least one of the SNP is an SNP found in the genetic code which encodes a IFIH1, such as IFIH1.2, IFIH1.3, or any combination hereof.

In one embodiment, the at least one of the SNP is an SNP found in the genetic code which encodes a DDX58, such as DDX58.2.

In one embodiment, the at least one of the SNP is an SNP found in the genetic code which encodes a NOD1, such as NOD1.2, NOD1.3, NOD1.4, or any combination hereof.

In one embodiment, the at least one of the SNP is an SNP found in the genetic code which encodes a NOD2, such as NOD2.3, NOD2.4, or any combination hereof.

It will be recognized that the SNPs referred to herein may be the polymorphisms which are analyzed in step b) of the method for the identification of polymorphisms of PRR, such as TLR, NLR, or RLR encoding genetic code which is correlated to a prognosis of a subject for the development of an immune response to a bio-agent, according to the invention.

In one embodiment, the at least one of the SNP is a SNP found in the genetic code which encodes a TLR selected from the group consisting of TLR2.2, TLR5.3, TLR6.3, TLR7.1, TLR8.1, TLR9.1, TLR10.4, TLR10.5, IFIH1.2, IFIH1.3, DDX58.2, NOD1.2, NOD1.3, NOD1.4, NOD2.3, and NOD2.4.

### Multiplexed Reactions

In one embodiment, step b) comprises determining the presence or absence of at least 2, (such as at least 2, at least 3, at least 4, at least 5, at least 6, at least 7, or at least 8) SNPs in the genetic code which encodes a PRR, such as TLR, NLR, and/or RLR, or more than one PRR, such as TLR, NLR, and/or RLR such as at least 2, (such as at least 3, at least 4, at least 5, at least 6, at least 7, or at least 8 PRRs, such as TLR, NLR, and/or RLR.

In one embodiment, step b) of the prognostic method comprises determining the presence or absence of at least five SNPs in the genetic code which encodes one or more PRRs, such as TLR, NLR, or RLR.

In one embodiment, the at least five SNPs are present in at least 3 independent PRRs, such as TLR, NLR, or RLR.

In one embodiment, step b) comprises determining the presence or absence of at least eight SNPs in the genetic code which encodes at least three independent PRRs, such as TLR, NLR, or RLR.

In one preferred embodiment, the determining the presence or absence (and/or copy number) of at least 2 SNPs referred to in step b) occurs concurrently (such as simultaneously within the same experiment/method), typically in the same ‘pot’ or reaction, i.e. a multiplexed reaction.

Suitably, step b) may comprise a multiplexed PCR reaction for the co-amplification of said at least two SNPs.

In one embodiment said at least 5, such as said at least 8 SNPs are detected or co-amplified concurrently (such as simultaneously within the same experiment/method).

In one embodiment, step b) comprises the following sequential steps:
i) a multiplexed PCR reaction in which the SNPs are amplified,
ii) an allele-specific primer extension reaction (ASPE) in which label moieties are incorporated into the ASPE-primers which match the genotype of the sample,
iii) isolating the extension reaction products into separate populations of individual SNP amplification products.

In one embodiment, the labeled moiety referred to in step ii) is a biotin label, such as a biotinylated nucleotide. Further alternative labels include phycoerythrin (PE)-labeled moieties (such as nucleotide). Alternatively, one could use radio-labeled moiety.

In one embodiment, step iii) Comprises a hybridisation based isolation of individual populations of SNP amplification products, such as bead-array hybridisation.

In one embodiment of the prognostic method according to the invention, the heterozygosity of the at least one SNP is determined.

It will be recognized that alternative methods of labeling the multiplex products other than ASPE, such as single base chain extension (SBCE), Oligonucleotide ligation assay (OLA), or alternatively the PCR products may be directly hybridised to (SNP specific) probe-coupled beads based on the presence or absence of the SNP.

SBCE differs from ASPE in several ways; the allele-specific primers 3′-ends overlap one of the nucleotides located right next to the SNP-loci on either the 3′- or the 5′-side of the SNP. When an allele-specific primer hybridizes to a SNP-locus the polymerase elongates it incorporating a biotinylated dideoxy-dNTP (ddNTP), this method has the advantage that a single allele-specific primer can be used to detect up to four different alleles at a given locus, the drawback being that the reaction has to be performed in four different tubes corresponding to the four possible nucleotides ddATP, ddCTP, ddGTP and ddTTP.

Oligonucleotide ligation assay (OLA): The OLA-assay is based on the ability of two oligonucleotides, one labeled the other allele-specific, to anneal immediately adjacent to each other on a complementary target DNA molecule. The two oligonucleotides are then joined covalently by the action of a DNA ligase, provided that the nucleotides at the junction are correctly base-paired. In this way only a primer matching the present allele at a polymorphic locus will be joined to the labeled oligonucleotide and hence emit detectable fluorescence.

Probe-bead based assay: In the probe-bead based assay a multiplex PCR is performed on the SNP-sites of interest with at least one of the primers in each primer-pair being labeled. An allele-specific probe overlapping a suitable area of the polymorphic locus is then prepared and coupled covalently to suitable microspheres. With all other than the perfectly matching PCR-product, the probe will form a loop because of the mismatching base-pair in the middle of the probe-PCR product hybridization complex and this significantly decreases the melting temperature of the complex ensuring that only perfectly hybridized oligonucleotides will remain attached to the probe and hence emit detectable fluorescence.

ASPE, SBCE, OLA and the probe-bead based assays are all suited for the Luminex platform, but different solid base supports such as microarray chips or possibly other beads available for FACS-cytometers etc. could easily be substituted for the Luminex platform. References for these assays can be found herein ([36]-[40]):

In a preferred embodiment, the method for the identification of polymorphisms of PRR, such as TLR, NLR, or RLR encoding genetic code which is correlated to a prognosis of a subject for the development of an immune response to a bio-agent is performed using a multiplexed reaction. This allows for the efficient identification of polymorphisms (such as SNPs) on numerous PRR, such as TLR, NLR, or RLR SNPs simultaneously, thereby allowing the identification of specific SNPs which correlate to a specific prognosis.

The multiplex reaction may comprise analysis of SNPs within the genetic codes which encode TLR1, TLR2, TLR3, TLR4, TLR5, TLR6, TLR7, TLR8, TLR9 and TLR 10.

The multiplex reaction may comprise analysis of SNPs within the genetic codes which encode TLR2, TLR4, TLR5, and TLR9.

The multiplex reaction may comprise analysis of SNPs within the genetic codes which encode TLR3, TLR7 and TLR8.

The multiplex reaction may comprise analysis of SNPs within the genetic codes which encode IFIH1, DDX58, NOD1, and NOD2.

The multiplex reaction may comprise analysis of SNPs within the genetic codes which encode NOD1 and NOD2.

The multiplex reaction may comprise analysis of SNPs within the genetic codes which encode the SNPs shown in Table 2, table 3, or in table 2 of WO 2007/025989.

The multiplex reaction may comprise analysis of SNPs within the genetic codes which encode TLR5, TLR7, TLR8 and TLR 9.

The multiplex reaction may comprise analysis of SNPs within the genetic codes which encode TLR-1, 2, 6 and 10.

The multiplex reaction may comprise analysis of SNPs within the genetic codes which encode TLR-1.

The multiplex reaction may comprise analysis of SNPs within the genetic codes which encode TLR-2.

The multiplex reaction may comprise analysis of SNPs within the genetic codes which encode TLR-6.

The multiplex reaction may comprise analysis of SNPs within the genetic codes which encode TLR-10.

The multiplex reaction may comprise analysis of SNPs within the genetic codes which encode TLR-4.

The multiplex reaction may comprise analysis of SNPs within the genetic codes which encode TLR-5.

The multiplex reaction may comprise analysis of SNPs within the genetic codes which encode

The multiplex reaction may comprise analysis of SNPs within the genetic codes which encode TLR-7, 8 and 9.

The multiplex reaction may comprise analysis of SNPs within the genetic codes which encode TLR-7.

The multiplex reaction may comprise analysis of SNPs within the genetic codes which encode TLR-8.

The multiplex reaction may comprise analysis of SNPs within the genetic codes which encode TLR-9.

The multiplex reaction may comprise analysis of SNPs within the genetic codes which encode IFIH1.

The multiplex reaction may comprise analysis of SNPs within the genetic codes which encode DDX58.

The multiplex reaction may comprise analysis of SNPs within the genetic codes which encode NOD1.

The multiplex reaction may comprise analysis of SNPs within the genetic codes which encode NOD2.

The multiplex reaction may comprise analysis of SNPs within the genetic codes which encode NOD1 and NOD2.

Therefore, it will be recognized that the SNPs referred to herein may be the polymorphisms which are analysed in step b) of the method for the identification of polymorphisms of PRR, such as TLR, NLR, or RLR encoding genetic code which is correlated to a prognosis of a subject for the development of an immune response to a bio-agent, according to the invention.

Comparing the Presence or Absence or Copy Number of the at Least One SNPs Identified in Step b) with Control Data

The prognosis is determined by comparing the SNP data obtained in step b) with control data. Typically the control data is obtained from either a subject which has developed the disease; and/or a subject which has developed the disease and has a history of failed treatment of said disease. In relation to bio-diagnostics the control data is suitably obtained from (ii) subjects which have a history of failed or incorrect diagnosis and/or (iv) subjects which have a history of successful diagnosis, in relation to the bio-diagnostics agent.

Suitably, the control data referred to in step c) is obtained by performing comparative SNP analysis on one or more subject groups selected from the subject groups consisting of:
i) One or more subjects which have developed the disease;
ii) One or more subjects which have developed the disease and have also history of failed treatment of said disease using the biopharmaceutical agent;
iii) One or more subjects which have not developed the disease;
iv) One or more subjects which have developed the disease but have shown a positive response to therapeutic treatment.

Suitably, the most useful control data is the data obtained from ii) and/or iv).

Suitably, the comparative SNP analysis may be performed either prior to, concurrently or subsequent to step c). It is recognized that the comparative SNP analysis may already have been performed prior to the claimed method, either within the context of the same experiment, or, as is more likely, by one or more previous experiments, the results of which, for example, may be available via publications or from third parties.

Clearly, by comparing the data obtained in step b) with the control data referred to in step c), the method of the present invention enables a determination of the likelihood of the success of the treatment of a disease or prevention of the development of a disease in the subject.

The invention further provides for a kit for use in the prognostic method according to the invention, said kit comprising means for detecting at least one SNP (SNP) in the genetic code which encodes for one or more Pattern recognition receptors (PRRs), such as TLR, NLR, or RLR.

The invention further provides for a kit for use in the prognostic method according to the invention, said kit comprising:
a) A means for detecting at least one SNP (SNP) in the genetic code which encodes for one or more Pattern recognition receptors (PRRs), such as TLR, NLR, or RLR;
b) A means for comparing the presence or absence of the at least one SNP identified in step a) with control data obtained from a subject which has developed the disease and has a history of failed treatment of said disease.

In some embodiments the kit comprises at least one primer set, such as a primer set according to table 4 or 5, such as a polynucleotide comprising a nucleotide sequence corresponding to any one sequence of SEQ ID NO:1-252; and optionally
one or more elements selected from
   i) a control sample, such as DNA-samples with known genotypes for the at least one polymorphic locus;
   ii) instructions for use;
   iii) a PCR-reagent mixture;
   iv) a piece of software capable of performing data analysis; and
   v) a biopharmaceutical according to the biopharmaceutical treatment.

In some embodiments at least one primer set according to the following table is used in the methods and kits according to the present invention:

| | | SEQ ID NOs for ASPE- | SEQ ID NOs for |
|---|---|---|---|
| | SEQ ID NOs for PCR-primers | primers | FlexMAP bead-sets |
| TLR2.2 | 3 + 4, 39 + 40 | 119 + 120, 171 + 172 | 18 + 41 |
| TLR5.3 | 11 + 12, 55 + 56 | 133 + 134, 187 + 188 | 21 + 22 |
| TLR6.3 | 65 + 66 | 197 + 198 | 69 + 93 |
| TLR7.1 | 17 + 18, 67 + 68 | 145 + 146, 199 + 200 | 65 + 47 |
| TLR8.1 | 25 + 26, 75 + 76 | 153 + 154, 207 + 208 | 90 + 40 |
| TLR9.1 | 13 + 14, 81 + 82 | 139 + 140, 215 + 216 | 64 + 82 |
| TLR10.4 + 5 | 89 + 90, 91 + 92 | 225 + 226, 227 + 228 | 99 + 83, 11 + 52 |
| CARD4.1 + 2 + 3 | 103 + 104, 105 + 106, 107 + 108 | 239 + 240, 241 + 242, | 5 + 99, 82 + 28, |
| | | 243 + 244 | 87 + 10 |
| CARD15.4 | 115 + 116 | 251 + 252 | 18 + 60 |

### Human Toll Like Receptors

Toll-Like Receptor (TLR): Toll-Like Receptors is a class of highly conserved type 1 trans-membrane proteins that form a key part of the innate immune system, and, in vertebrates are able to stimulate activation of the adaptive immune system, thereby linking the innate and acquired immune responses. Most mammalian species have between 10-15 Toll-like receptor proteins, and ten have been identified in humans (TLR1-TLR10). Reference sequences for TLRs are provided as SEQ IDs No 1-10 of WO 2007/025989 (which are hereby incorporated by reference).

As used herein the term ‘toll-like receptor’ refers to one of the following proteins which are available via Genbank, and include allelic variants thereof (i.e. variants which exist at the same (allelic) genomic position, but comprise one or more sequence polymorphisms, such as single nucleotide polymorphisms, but suitably retain at least 95% homology (such as at least 96, 97, 98, or 99% homology) at the DNA level to the following sequences.

At the time of preparing this specification, the following Genbank references (NCBI) were available and refer to human toll like receptors (1-10) proteins and are hereby incorporated by reference. The respective nucleotide sequences, available from NCBI are also hereby incorporated by reference.

### TLR1—AAI09095 AAI09094 AAH89403 EAW92901 AAC34137

### TLR-2 AAM23001 AAH33756 EAX04953 EAX04952 AAC34133

### TLR-3 AAH96335 AAH96333 AAC34134 AAH94737 EAX04628 ABE01399 AAH59372

### TLR-4 AAI17423 AAF07823 AAF05316 AAC34135 AAF89753

### TLR-5 EAW93263 EAW93262 AAI09119 AAI09120 AAC34136

### TLR-6 AAI11756 EAW92902

### TLR-7 AAF78035 AAF60188 EAW98807 AAH33651 AAQ88659

### TLR-8 AAI01076 AAI01077 AAI01075 AAI01078 AAF78036 AAF64061 EAW98809 EAW98808 AAQ88663

### TLR-9 AAF72189 AAF61307 AAF78037 EAW65191 AAH32713 AAQ89443 AAF72190 AAF72190 AAG01736 AAG01735 AAG01734

### TLR-10 AAK26744 EAW92900 EAW92899 EAW92898 EAW92897 AAI09113 AAI09112 AAH89406 AAQ88667.

Further TLRs, their genomic DNA, mRNA and protein sequences are provided in table 1 of WO 2007/025989 (table 1 of WO 2007/025989, and the respective sequences referred to therein and as disclosed in WO 2007/025989, are hereby incorporated by reference).

### TLR Polymorphisms

The polymorphisms include those referred to in table 2 of WO 2007/025989, and table 2 of WO 2007/025989 is hereby incorporated by reference.

TABLE 2
Further TLR1-10 polymorphisms causing
amino-acid substitutions or changes to
the promoter, 3′UTR, 5′UTR, or
intronic sequences.

| SNP | rs# | Mutation |
|---|---|---|
| TLR1 | rs5743611 | Arg80Thr |
| | rs4833095 | Asn248Ser |
| | rs3923647 | His305Leu |
| | rs5743618 | Ser602Ile |
| TLR2 | rs1898830 | Promotor |
| | rs5743704 | Pro631His |
| | rs5743708 | Arg753Gln |
| TLR3 | rs3775291 | Leu412Phe |
| TLR4 | rs7873784 | 3′UTR |
| | rs16906079 | Thr175Ala |
| | rs4986790 | Asp299Gly |
| | rs5031050 | Phe342Tyr |
| | rs4986791 | Thr399Ile |
| TLR5 | rs764535 | Thr83Ile |
| | rs5744168 | Arg392STOP |
| | rs2072493 | Asn592Ser |
| | rs5744174 | Phe616Leu |
| | rs5744176 | Asp694Gly |
| TLR6 | rs5743815 | Val427Ala |
| | rs5743813 | His345Tyr |
| | rs5743810 | Ser249Pro |
| TLR7 | rs2302267 | Intron 1 |
| | | (exon/intron |
| | | boundary) |
| | rs179008 | Gln11Leu |
| | rs5743781 | Ala448Val |
| | rs3853839 | 3′UTR |
| TLR8 | rs5741883 | Promoter |
| | rs3764879 | Promoter |
| | rs3764880 | Met1Val |
| | | (Startcodon) |
| | rs5744088 | Exon |
| TLR9 | rs187084 | Promoter |
| | rs5743836 | Promoter |
| | rs5743837 | Promoter |
| | rs5743841 | Exon |
| | rs5743844 | Pro99Leu - |
| | | P99L |
| | rs5743847 | Exon |
| TLR10 | rs4129008 | Arg800Gln |
| | rs4129009 | Ile775Val |
| | rs11466658 | Arg525Trp |
| | rs11466657 | Ile473Thr |
| | rs11466656 | Arg469Gly |
| | rs11096955 | Ile369Leu |
| | rs11466653 | Met326Thr |
| | rs11466651 | Val298Ile |
| | rs11096957 | Asn241His |
| | rs11466650 | Leu167Pro |
| | rs11466649 | Ala163Ser |

See table 3 for nomenclature of specific SNPs.

In one embodiment, step b) comprises the determination of the presence, absence or copy number of at least one SNP within the genetic code which encodes a TLR selected from the group consisting of TLR1, TLR2, TLR3, TLR4, TLR5, TLR6, TLR7, TLR8, TLR9 and TLR10.

In one embodiment, step b) comprises the determination of the presence, absence or copy number of at least one SNP within the genetic code which encodes a NLR selected from the group consisting of NOD1 (CARD4) and NOD2 (CARD15).

In one embodiment, step b) comprises the determination of the presence, absence or copy number of at least one SNP within the genetic code which encodes a RLR selected from the group consisting of MDA5 (IFIH1) and RIG-I (DDX58).

In one embodiment, the at least one SNP is selected from the group consisting of the SNPs shown in Table 2, table 3, or in table 2 of WO 2007/025989.

In one embodiment, the at least one SNP is a SNP found in the genetic code which encodes a TLR selected from the group consisting of TLR5, TLR7, TLR8 and TLR9.

In one embodiment, the at least one SNP is a SNP found in the genetic code which encodes a PRR selected from the group consisting of IFIH1 (MDA5) and DDX58 (RIG-I).

In one embodiment, the at least one SNP is a SNP found in the genetic code which encodes a PRR selected from the group consisting of NOD1 (CARD4) and NOD2 (CARD15).

Preferred TLR polymorphisms include polymorphisms present in TLR9. As shown in the examples the SNP located in the promoter region (TLR 9.1) was found to be associated to the response to treatment of rheumatoid arthritis using either of Infliximab and Adalimumab. Reference is made to FIG. 2.

Preferred TLR polymorphisms include polymorphisms present in TLR7. As shown in the examples the SNP located in TLR7 (TLR7.1) was found to be associated to the response to treatment of rheumatoid arthritis using Adalimumab.

Preferred TLR polymorphisms include polymorphisms present in TLR8. As shown in the examples the SNP located in TLR8 (TLR8.1) was found to be associated to the response to treatment of rheumatoid arthritis using Adalimumab.

Preferred TLR polymorphisms include polymorphisms present in TLR2. As shown in table 7 the SNP located in TLR2 (TLR2.2) was found to be associated to the development of neutralizing antibodies against beta-interferon in multiple sclerosis.

Preferred TLR polymorphisms include polymorphisms present in TLR6. As shown in table 7 the SNP located in TLR6 (TLR6.3) was found to be associated to the development of neutralizing antibodies against beta-interferon in multiple sclerosis and to the severity (MSSS) of the disease.

Preferred TLR polymorphisms include polymorphisms present in TLR8. As shown in table 7 the SNP located in TLR8 (TLR8.1) was found to be associated to the development of neutralizing antibodies against beta-interferon in multiple sclerosis.

Preferred TLR polymorphisms include polymorphisms present in TLR9. As shown in table 7 the SNP located in TLR9 (TLR9.1) was found to be associated to the development of neutralizing antibodies against beta-interferon in multiple sclerosis.

Preferred TLR polymorphisms include polymorphisms present in TLR10. As shown in table 7 the SNPs located in TLR10 (TLR10.4 and TLR10.5) was found to be associated to the development of neutralizing antibodies against beta-interferon in multiple sclerosis.

Preferred TLR polymorphisms include polymorphisms present in DDX58 (RIG-I). As shown in table 7 the SNP located in DDX58 (DDX58.2) was found to be associated to the development of neutralizing antibodies against beta-interferon, the rate of steroid-requiring attacks, and to interferon-respondership in multiple sclerosis.

Preferred TLR polymorphisms include polymorphisms present in NOD1 (CARD4). As shown in table 7 the SNPs located in NOD1 (NOD1.3 and NOD1.4) was found to be associated to the time to first attack after initiation of interferon treatment and to interferon-respondership in multiple sclerosis.

Preferred TLR polymorphisms include polymorphisms present in NOD2 (CARD15). As shown in table 7 the SNP located in NOD2 (NOD2.4) was found to be associated to the development of neutralizing antibodies against beta-interferon and to the time to first attack after initiation of interferon treatment in multiple sclerosis.

The method according to the invention may, for example, be used for identifying likely primary, non-, or low-responders of treatment with the biopharmaceutical. These may, for example, be patients that happen to have an innate immune response to the biopharmaceutical agents, or specific biopharmaceutical agents. Where the bio-agent is a diagnostic antibody, the identification of primary non- or -low responders can ensure the selection of a suitable diagnostic agent for each individual patient.

The method according to the invention may, for example, be used for identifying patients with secondary response failure. Secondary response failures can be asymptomatic, i.e. the only symptoms are that the treatment has become less effective or even non-effective. In this instance the use of the method according to the invention can be used to identify the likelihood of the development of secondary response failure before the start of therapy or during therapy but prior to the patient or medical practitioner has noticed that the treatment is less effective. A higher dosage of treatment may be applied to ensure the correct in vivo concentration is achieved, or alternative treatments can be selected, or a combination thereof. When the bio-agent is a diagnostic, the development of secondary response failure can be particularly catastrophic. Radio-labeled monoclonal antibodies are routinely used in the monitoring of diseases such as cancers, and some infectious diseases, where it is important to determine the size and/or location of the disease/agent—for example in identifying the presence/location of any secondary metastases. When the development of response failure (either primary or secondary) occurs unnoticed, the patient may be given the ‘all clear’—i.e. a false negative result, this can lead to the cessation of treatment and the latter re-appearance of the disease, often in a far more developed and possibly untreatable condition.

A further category of response failure is the development of (e.g. secondary) response failure associated with adverse side effects. Although rare, the development of a host-immune response in a subject can be accompanied by deleterious or unpleasant side effects. These may be caused by the development of antibodies which recognize the biopharmaceutical, but may then fail to distinguish with other host immunoglobulins.

In a highly preferred embodiment, the single light chain subtype bio-agent, such as biopharmaceutical/biodiagnostic, is a monoclonal antibody which comprises the lambda or kappa single light chain sub-type. In one embodiment, the monoclonal antibody comprises either lambda or kappa single light chain sub-types, but not both.

In one embodiment, the biopharmaceutical/biodiagnostic is either a humanised or a fully-human biopharmaceutical, such as a humanised or a fully-human biopharmaceutical monoclonal antibody.

The term ‘humanised’ refers to biopharmaceuticals which are derived, at least in part from a protein (sequence) which is not found in the species to which the subject belongs (typically human), but which has been modified to eliminate non-human epitopes which are or may be recognised as foreign by the human (typically acquired) immune system. Humanised biopharmaceuticals may for example be fusion proteins between a variable region obtained from a non-human source within the context of a human derived immunoglobulin protein sequence. A fully-human biopharmaceutical is derived from the (or a) human sequence.

In one embodiment, the biopharmaceutical is an antibody which specifically binds a target selected from the group consisting of: TNF-alpha, TNF-beta, IL-1, IL-6, GM-CSF, and VEGF, preferably TNF-alpha.

Clearly one major application area for the method of the present invention is in the selection and management of treatment regimes which involve the administration of biopharmaceuticals to patients. Therefore, the prognostic method, as described herein, can be incorporated into a method of treatment of a disease or a disorder. By performing the prognostic method, the selection and/or administration of the biopharmaceutical agent can be tailored to ensure maximum therapeutic benefit to the patient, whilst ensuring cost effective use of expensive biopharmaceutical agents.

The method according to the invention may be used to determine which therapy (such as biopharmaceutical) is used, or to optimise the dosage regime of the biopharmaceutical.

The therapeutic method may involve a periodic assessment of the serum concentration or bioavailability of the biopharmaceutical in the patient.

The invention provides for a method of determining whether the lack of treatment response in a patient is likely to be due to the ability of the patient to produce immunoglobulins directed against the biopharmaceutical.

The invention provides for a method of selecting the appropriate drug treatment for a patient suffering from a disease which is treatable with a biopharmaceutical (using the method steps referred to herein).

The invention provides for a prognostic method for the determination of the likelihood of whether a patient will develop secondary response failure to a biopharmaceutical (using the method steps referred to herein).

### Suitable Biopharmaceuticals and Disorders

An extensive list of biopharmaceuticals therapeutics in clinical development and approved products are disclosed in the 2006 PhRMA Report entitled ‘418 Biotechnology Medicines in Testing Promise to Bolster the Arsenal Against Disease’.

A preferred class of biopharmaceuticals are anti-TNF-alpha single chain monoclonal antibodies which are used in treatment of numerous autoimmune diseases, such as—rheumatoid arthritis, juvenile idiopathic arthritis, ankylosing spondylitis (Bechterew's disease), inflammatory bowel diseases (Crohn's diseases and ulcerative colitis), severe psoriasis, chronic uveitis, severe sarcoidosis and Wegener's granulomatosis, and other chronic immunoinflammatory diseases.

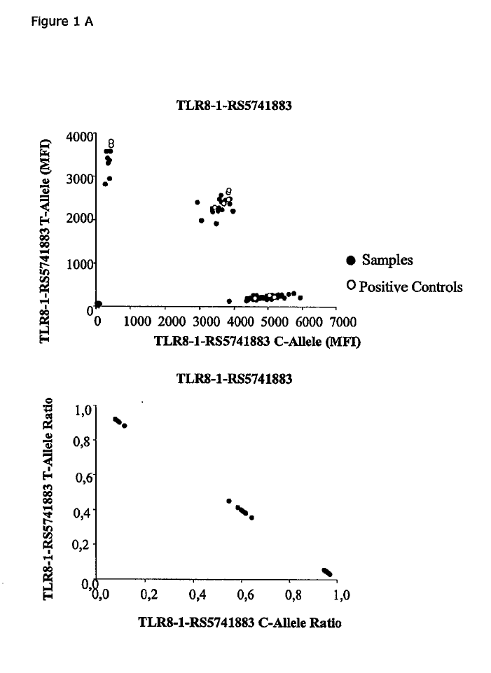

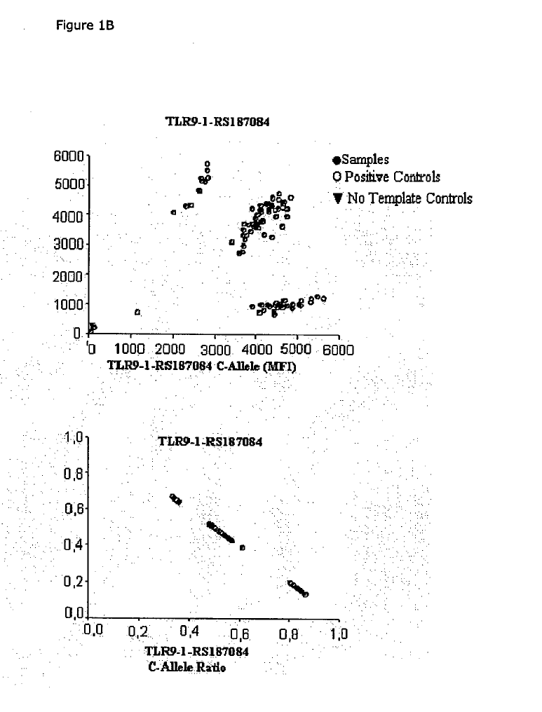

One particularly preferred group of biopharmaceuticals are the anti-TNFalpha monoclonal antibodies, which include (see FIG. 1) Remicade™ (infliximab), a mouse-human IgG1-kappa anti-TNF-alpha monoclonal antibody, 2) Enbrel™ (etanercept), a fusion protein of human TNF receptor 2 and human IgG1, and 3) Humira™ (adalimumab), a fully human IgG1-kappa anti-TNF-alpha monoclonal antibody. Two other anti-TNF-alpha antibody constructs have shown promise in pivotal phase III trials in patients with some of the same diseases: 4) Cimzia™ CDP870 (certolizumab pegol), a PEGylated Fab fragment of a humanized anti-TNF-alpha monoclonal antibody, and 5) CNTO 148 (golimumab), a fully human IgG1-kappa anti-TNF-alpha monoclonal antibody.

Another particularly preferred group of biopharmaceuticals are the recombinant interferons, which include Betaferon™ (interferon beta-1b), Betaseron™ (interferon beta-1b), Avonex™ (interferon beta-1a), and Rebif™ (interferon beta-1a).

### Diseases:

The following diseases are treated using biopharmaceuticals, and as such the disease, as referred to in the method according to the invention, may be selected from the group consisting of:

Infectious diseases, such as respiratory syncytial virus (RSV), HIV, anthrax, candidiasis, staphylococcal infections, hepatitis C, sepsis;
Autoimmune diseases, such as rheumatoid arthritis, Crohn's disease, B-cell non hodgkin's lymphoma, Multiple sclerosis, SLE, ankylosing spondylitis, lupus, psoriatic arthritis, erythematosus;
Inflammatory disorders such as rheumatoid arthritis (RA), juvenile idiopathic arthritis, ankylosing spondylitis (Bechterew's disease), inflammatory bowel diseases (Crohn's diseases and ulcerative colitis), severe psoriasis, chronic uveitis, sarcoidosis, Wegener's granulomatosis, and other diseases with inflammation as a central feature;
Blood disorders, such as sepsis, septic shock, paroxysmal nocturnal hemoglobinuria, and hemolytic uremic syndrome (also included under infectious diseases);
Cancers, such as colorectal cancer, non-Hodgkin's lymphoma, B-cell chronic lymphocytic leukemia, anaplastic large-cell-lymphoma, squamous cell cancer of the head and neck, treatment of HER2-overexpressing metastatic breast cancer, acute myeloid leukemia, prostate cancer (e.g. adenocarcinoma), small-cell lung cancer, thyroid cancer, malignant melanoma, solid tumors, breast cancer, early stage HER2-positive breast cancer, first-line non-squamous NSCLC cancers, AML, hairy cell leukemia, neuroblastoma, renal cancer, brain cancer, myeloma, multiple myeloma, bone metastases, SCLC, head/neck cancer, first-line pancreatic, SCLC, NSCLC, head and neck cancer, hematologic and solid tumors, advanced solid tumors, gastrointestinal cancer, pancreatic cancers, cutaneous T-cell lymphoma, non-cutaneous T-cell lymphoma, CLL, ovarian, prostate, renal cell cancers, mesothelin-expressing tumors, glioblastoma, metastatic pancreatic, hematologic malignancies, cutaneous anaplastic large-cell MAb lymphoma, AML, myelodysplastic syndromes;
Cardiovascular diseases, such as atherosclerosis acute myocardial infarction, cardiopulmonary bypass, angina, stroke;
Metabolic disorders such as diabetes, such as type-1 and type-2 diabetes mellitus;
Digestive disorders, such as Crohn's disease, C. difficile disease, ulcerative colitis;
Eye disorders such as uveitis;
Genetic Disorders such as paroxysmal nocturnal hemoglobinuria (PNH);
Neurological Disorders such as osteoarthritis pain and Alzheimer's disease;
Respiratory Disorders such as respiratory diseases, asthma, chronic obstructive pulmonary disorders (COPD, nasal polyposis, pediatric asthma);
Skin diseases, such as psoriasis, including chronic moderate to severe plaque psoriasis, and eczma; and
Transplant rejection, such as acute and chronic rejections of kidneys, heart, lungs, liver, pancreas and pancreatic islets and bone marrow, or Graft-versus-host disease in bone-marrow transplantations.

In one embodiment, the disease is selected from the group consisting of: rheumatic diseases (rheumatoid arthritis (RA), ankylosing spondylitis, etc), inflammatory bowel diseases (Crohn's disease, ulcerative colitis), inflammatory skin diseases (psoriasis, eczema, etc), inflammatory diseases of the brain and peripheral nerves (multiple sclerosis, various neuropathies, etc), vascular inflammatory diseases (arteriosclerosis), periodontitis, and inflammatory diseases of muscles (heart and skeletal), eyes, lungs, liver, kidneys, bone and endocrine organs, incl. diabetes.

In one embodiment the disease is selected from one or more of the above groups or specific diseases/disorder. Preferred diseases are diseases where repeated dosages of the bio-agent are used, such as autoimmune diseases. Particularly preferred disorders are chronic autoimmune conditions.

TABLE 1
Therapeutic and diagnostic monoclonal antibodies (Approved are underlined)

| | | |
|---|---|---|
| Product Name | Sponsor | Indication |

Infectious diseases

| | | |
|---|---|---|
| Synagis ® | MedImmune | prevention of respiratory syncytial virus (RSV) |
| palivizumab | | |
| anti-HIV-1 MAb | Polymun Scientific | HIV infection treatment |
| | Vienna, Austria | |
| CCR5 MAb | Human Genome | HIV infection |
| | Sciences Rockville, MD | |
| Cytolin ® | CytoDyn | HIV infection |
| anti-CD8 MAb | Santa Fe, NM | |
| NM01 | SRD Pharmaceuticals | HIV infection |
| | Los Angeles, CA | |
| PRO 140 | Progenics | HIV infection |
| | Pharmaceuticals | |
| | Tarrytown, NY | |
| TNX | 355 Tanox MAb HIV | 355 Tanox MAb HIV infection Phase II |
| | infection Phase II | |
| ABthrax ™ | Human Genome | anthrax |
| raxibacumab | Sciences | |
| Anthim ™ | Elusys Therapeutics | anthrax |
| (ETI-204) | | |
| (Orphan Drug) | | |
| anti-hsp90 MAb | NeuTec Pharma | candidiasis |
| anti-staph MAb | MedImmune | Prevention of staphylococcal infections |
| Aurexis | Inhibitex | prevention and treatment of S. aureus |
| tefibazumab | | bacteremia |
| bavituximab | Peregrine | hepatitis C treatment |
| | Pharmaceuticals | |
| MDX-1303 | Medarex | anthrax |
| | PharmAthene | |
| Numax ™ | MedImmune | RSV |
| motavizumab | | |
| Tarvacin ™ | Peregrine | hepatitis C |
| bavituximab | Pharmaceuticals | |
| XTL 6865 | XTL Biopharmaceuticals | hepatitis C |

Autoimmune disorders

| | | |
|---|---|---|
| Humira ® | Abbott Laboratories | rheumatoid arthritis |
| adalimumab | | |
| Remicade ™ | Centocor | Crohn's disease, rheumatoid arthritis |
| infliximab | | |
| Rituxan ® | Genentech | B-cell non hodgkin's lymphoma, relapse in |
| ritiximab | Biogen Idec | patients following rituxan treatment. |
| | | Rheumatoid arthritis |
| Tysabri ® | Biogen Idec | Multiple sclerosis |
| natalizumab | | |
| ABT 874 | Abbott Laboratories | multiple sclerosis, |
| Actemra | Roche | rheumatoid arthritis, |
| AME 527 | Applied Molecular | rheumatoid arthritis |
| AMG 108 | Amgen | rheumatoid arthritis |
| AMG 714 | Amgen | rheumatoid arthritis |
| anti-CD16 MAb | MacroGenics | immune thrombocytopenic |
| CNTO 1275 | Centocor | multiple sclerosis |
| | Horsham, PA | |
| daclizumab | PDL BioPharma | multiple sclerosis |
| (anti-CD25 MAb) | Fremont, CA | (see also respiratory) |
| | Biogen Idec | |
| | Cambridge, MA | |
| denosumab | Amgen | rheumatoid arthritis |
| (AMG 162) | Thousand Oaks, CA | |
| ETI-201 | Elusys Therapeutics | SLE |
| | Pine Brook, NJ | |
| golimumab | Centocor | rheumatoid arthritis |
| | Horsham, PA | |
| HuMax-CD20 | Genmab | rheumatoid arthritis |
| (ofatumumab) | Princeton, NJ | |
| Humira ® | Abbott Laboratories | ankylosing spondylitis |
| adalimumab | | juvenile rheumatoid arthritis |
| HuZAF ™ | PDL BioPharma | rheumatoid arthritis |
| fontolizumab | Fremont, CA | |
| | Biogen Idec | |
| | Cambridge, MA | |
| IMMU-106 | Immunomedics | autoimmune disease |
| (hCD20) | Morris Plains, NJ | |
| LymphoStat-B ™ | Human Genome | rheumatoid arthritis, SLE |
| belimumab | Sciences | |
| | Rockville, MD | |
| MEDI-545 | Medarex | lupus |
| (MDX-1103) | Princeton, NJ | |
| | MedImmune | |
| | Gaithersburg, MD | |
| MLN 1202 | Millennium | multiple sclerosis |
| | Pharmaceuticals | |
| | Cambridge, MA | |
| ocrelizumab | Genentech | rheumatoid arthritis |
| (2nd anti-CD20) | South San Francisco, | |
| (R1594) | CA | |
| | Biogen Idec | |
| | Cambridge, MA | |
| | Roche | |
| | Nutley, NJ | |
| OKT3-gamma-1 | Johnson & Johnson | psoriatic arthritis |
| | Pharmaceutical | |
| | Research | |
| | & Development | |
| | Raritan, NJ | |
| Rituxan ® | Genentech | rheumatoid arthritis |
| rituximab | South San Francisco, | (DMARD inadequate |
| | CA | responders), lupus, |
| | Biogen Idec | primary progressive |
| | Cambridge, MA | multiple sclerosis, SLE |
| | | (see also cancer) relapsing-remitting |
| | | multiple sclerosis |
| TRX 1 | TolerRx | cutaneous lupus |
| (anti-CD4) | Cambridge, MA | erythematosus |

Blood disorders

| | | |
|---|---|---|
| ReoPro ® | Centocor | anti-platelet prevention of blood clots (PTCA), |
| abciximab | Eli Lilly | angina (PTCA) |
| urtoxazumab | Teijin Pharma | hemolytic uremic |
| Afelimomab | Abbot Laboratories | Sepsis, septic shock |
| Eculizumab | Alexion | Paroxysmal nocturnal hemoglobinurea. |
| | Pharmaceuticals | |

Cancer

| | | |
|---|---|---|
| Avastin ™ | Genentech | metastatic colorectal cancer |
| bevacizumab | | |
| Bexxar ® | GlaxoSmithKline | non-Hodgkin's lymphoma |
| tositumomab, | | |
| iodine I 131 | | |
| tositumomab | | |
| Campath ® | Berlex Laboratories | B-cell chronic lymphocytic leukemia |
| Alemtuzumab | Genzyme | |
| Erbitux ™ | Bristol-Myers Squibb | colorectal cancer |
| cetuximab | Medarex | squamous cell cancer of the head and neck |
| Herceptin ® | Genentech | treatment of HER2-overexpressing metastatic |
| Trastuzumab | | breast cancer |
| Mylotarg ™ | Wyeth | Acute myeloid leukemia |
| gemtuzumab | | |
| ozogamicin | | |
| OncoScint ® | CYTOGEN | detection, staging and follow-up of colorectal |
| CR/OV | | cancers |
| satumomab | | |
| pendetide | | |
| ProstaScint ® | CYTOGEN | detection, staging and follow-up of prostate |
| capromab | | adenocarcinoma |
| pentetate | | |
| Rituxan ® | Genentech | B-cell non hodgkin's lymphoma, relapse in |
| ritiximab | Biogen Idec | patients following rituxan treatment. |
| Verluma ® | DuPont | detection of small-cell lung cancer |
| Nofetumomab | Pharmaceuticals | |
| Zevalin ™ | IDEC Pharmaceuticals | Non-hodgkin's lymphoma |
| ibritumomab | | |
| tiuxetan | | |
| 1311-huA33 | Life Science | colorectal cancer |
| | Pharmaceuticals | |
| | Greenwich, CT | |
| 1D09C3 | GPC Biotech | relapsed/refractory |
| | Waltham, MA | B-cell lymphomas |
| AGS-PSCA MAb | Agensys | prostate cancer |
| | Santa Monica, CA | |
| | Merck | |
| | Whitehouse Station, NJ | |
| AMG 102 | Amgen Thousand Oaks, | cancer |
| | CA | |
| AMG 479 | Amgen Thousand Oaks, | cancer |
| | CA | |
| AMG 623 | Amgen Thousand Oaks, | B-cell chronic lymphocytic leukemia (CLL) (see |
| | CA | also autoimmune) |
| AMG 655 | Amgen Thousand Oaks, | cancer |
| | CA | |
| AMG 706 | Amgen Thousand Oaks, | imatinib-resistant GIST, advanced thyroid |
| | CA | cancer |
| anti-CD23 MAb | Biogen Idec Cambridge, | CLL |
| | MA | |
| anti-CD80 MAb | Biogen Idec Cambridge, | non-Hodgkin's B-cell lymphoma |
| | MA | |
| anti-idiotype | Viventia Biotech | malignant melanoma |
| cancer vaccine | Toronto, Ontario | |
| anti-lymphotoxin | Biogen Idec Cambridge, | solid tumors |
| beta receptor | MA | |
| MAb | | |
| anti-PEM MAb | Somanta | cancer |
| | Pharmaceuticals Irvine, | |
| | CA | |
| anti-Tac(Fv)- | National Cancer | leukemia, lymphoma |
| PE38 | Institute Bethesda, MD | |
| immunotoxin | | |
| Avastin ® | Genentech | relapsed metastatic |
| bevacizumab | South San Francisco, | colorectal cancer |
| | CA | first-line metastatic breast, |
| | | first-line non-squamous NSCLC cancers |
| AVE 9633 | sanofi-aventis | AML |
| maytansin- | Bridgewater, NJ | |
| loaded | | |
| anti-CD33 MAb | | |
| bavituximab | Peregrine | solid cancers (see also infectious) |
| | Pharmaceuticals Tustin, | |
| | CA | |
| CAT 3888 | Cambridge Antibody | hairy cell leukemia |
| | Technology | |
| chimeric MAb | National Cancer | neuroblastoma |
| | Institute | |
| CNTO 328 | Centocor | renal cancer |
| Cotara ™ | Peregrine | brain cancer |
| | Pharmaceuticals | |
| bivatuzumab | Boehringer Ingelheim | cancer |
| | Pharmaceuticals | |
| | Ridgefield, CT | |
| CP-751,871 | Pfizer | multiple myeloma |
| CS 1008 | Daiichi Sankyo | cancer |
| | Sankyo Pharma | |
| | Development | |
| | Parsippany, NJ | |
| BrevaRex ™ | ViRexx | breast cancer, multiple |
| antibody-based | Edmonton, Alberta | myeloma |
| immunotherapy | | |
| denosumab | Amgen | bone loss induced by hormone ablation therapy |
| | | for breast or prostate |
| | | cancer, prolonging bonemetastases- |
| | | free survival |
| | | (see also autoimmune, other) |
| | | bone metastases in |
| | | breast cancer |
| ecromeximab | Kyowa Hakko USA | malignant melanoma |
| EMD 273063 | EMD Lexigen | solid tumors malignant melanoma, |
| | | neuroblastoma, SCLC |
| Erbitux ™ | Bristol-Myers Squibb | head/neck cancer, first-line pancreatic, first-line |
| | | NSCLC, -second-line NSCLC, first |
| | | line colorectal, second-line |
| | | colorectal cancers |
| GMK | Progenics | prevention of recurrence following surgery to |
| | Pharmaceuticals | remove primary melanoma |
| | | in high-risk patients |
| Campath ® | National Cancer | leukemia, lymphoma |
| alemtuzumab | Institute | |
| | Bethesda, MD | |
| | Berlex Laboratories | |
| | Montville, NJ | |
| Herceptin ® | Genentech | early stage HER2-positive |
| trastuzumab | South San Francisco, | breast cancer |
| | CA | first-line metastatic HER2- |
| | | positive breast cancer in combination with |
| | | Taxotere ® |
| HGS-ETR1 | Human Genome | hematologic and |
| | Sciences | solid tumors |
| | Rockville, MD | |
| HGS-ETR2 | Human Genome | hematologic and |
| (mapatumumab) | Sciences | solid tumors |
| | Rockville, MD | |
| HGS-TR2J | Human Genome | advanced solid tumors |
| | Sciences | |
| | Rockville, MD | |
| HuC242-DM4 | ImmunoGen | colorectal, gastrointestinal, |
| | Cambridge, MA | NSCLC, pancreatic |
| | | cancers |
| HuMax-CD4 | Genmab | cutaneous T-cell |
| (zanolimumab) | Princeton, NJ Serono | lymphoma |
| | Rockland, MA | non-cutaneous T-cell |
| | | lymphoma |
| HuMax-CD20 | Genmab | CLL, non-Hodgkin's |
| (ofatumumab) | Princeton, NJ | lymphoma |
| | | (see also autoimmune) |
| HuMax-EGFr | Genmab | head and neck cancer |
| | Princeton, NJ | |
| huN901-DM1 | ImmunoGen | SCLC |
| | Cambridge, MA | multiple myeloma |
| ipilimumab | Bristol-Myers Squibb | melanoma monotherapy |
| (MDX-010) | Medarex, Princeton, | leukemia, lymphoma, ovarian, prostate, renal |
| | | cell cancers |
| | | melanoma |
| | | (MCX-010 +/−DTIC) |
| | | second-line metastatic |
| | | melanoma |
| | | (MDX-010 disomotide/ |
| | | overmotide MDX-1379) |
| M195-bismuth | Actinium | AML |
| 213 conjugate | Pharmaceuticals | |
| | Florham Park, NJ | |
| M200 | PDL BioPharma | advanced solid tumors |
| (volociximab) | Fremont, CA Biogen | |
| | Idec Cambridge, MA | |
| MAb HeFi-1 | National Cancer | lymphoma, non-Hodgkin's lymphoma |
| | Institute Bethesda, MD | |
| MDX-060 | Medarex Princeton, NJ | Hodgkin's disease, anaplastic large-cell- |
| (iratumumab) | | lymphoma |
| MDX-070 | Medarex Princeton, NJ | prostate cancer |
| MDX-214 | Medarex | EGFR-expressing cancers |
| | Princeton, NJ | |
| MEDI-507 | MedImmune | T-cell lymphoma |
| siplizumab | Gaithersburg, MD | infections |
| MEDI-522 | MedImmune | melanoma, prostate cancer |
| | Gaithersburg, MD | solid tumors |
| | National Cancer | |
| | Institute | |
| | Bethesda, MD | |
| | MedImmune | |
| | Gaithersburg, MD | |
| MORAb 003 | Morphotek | ovarian cancer |
| | Exton, PA | |
| MORAb 009 | Morphotek | mesothelin-expressing |
| | Exton, PA | tumors |
| neuradiab | Bradmer | glioblastoma |
| | Pharmaceuticals | |
| | Louisville, KY | |
| nimotuzumab | YM Biosciences | metastatic pancreatic, |
| (Orphan Drug) | Mississauga, Ontario | NSCLC |
| ocrelizumab | Genentech | hematologic malignancies |
| (2nd anti-CD20) | South San Francisco, | (see also autoimmune) |
| (R1594) | CA | |
| | Biogen Idec | |
| | Cambridge, MA | |
| | Roche | |
| | Nutley, NJ | |
| Omnitarg ™ | Genentech | ovarian cancer |
| pertuzumab | South San Francisco, | |
| | CA | |
| OvaRex ® | ViRexx MAb Edmonton, | ovarian cancer |
| oregovomab | Alberta | |
| PAM 4 | Merck Whitehouse | pancreatic cancer |
| | Station, NJ | |
| panitumumab | Abgenix | colorectal cancer |
| (rHuMAb-EGFr) | | |
| Proleukin ® | Chiron Emeryville, CA | Non-hodgkin's lymphoma |
| PSMA | Progenics | prostate cancer |
| | Pharmaceuticals | |
| | Tarrytown, NY | |
| R1550 | Roche Nutley, NJ | metastatic breast cancer |
| RadioTheraCIM | YM BioSciences | glioma |
| | Mississauga, Ontario | |
| RAV 12 | Raven Biotechnologies cancer | |
| | South San Francisco, | |
| | CA | |
| Rencarex ® | Wilex Munich, Germany | renal cancer |
| G250 | | |
| Rituxan ® | Genentech | indolent non-Hodgkin's |
| rituximab | South San Francisco, | lymphoma induction |
| | CA | therapy |
| | Biogen Idec | (see also autoimmune) |
| | Cambridge, MA | relapsed or refractory CLL |
| SGN-30 | Seattle Genetics | cutaneous anaplastic large- |
| (Orphan Drug) | Bothell, WA | cell MAb lymphoma, |
| | | systemic anaplastic large- |
| | | cell lymphoma, Hodgkin's |
| | | disease |
| SGN-33 | Seattle Genetics | AML, myelodysplastic |
| (lintuzumab) | Bothell, WA | syndromes |
| SGN-40 | Seattle Genetics | CLL |
| | Bothell, WA | multiple myeloma, non- |
| | | Hodgkin's lymphoma |
| sibrotuzumab | Life Science | colorectal, head and neck, |
| | Pharmaceuticals | lung cancers |
| | Greenwich, CT | |
| Tarvacin ™ | Peregrine | solid tumors |
| bavituximab | Pharmaceuticals | (see also infectious) |
| | Tustin, CA | |
| ticilimumab | Pfizer New York, NY | metastatic melanoma |
| | | prostate cancer |
| TNX-650 | Tanox Houston, TX | Hodgkin's disease |
| Zevalin ™ | National Cancer | leukemia, lymphoma non-Hodgkin's lymphoma |
| ibritumomab | Institute Bethesda | |
| tiuxetan | Biogen, | |

Cardiovascular disease

| | | |
|---|---|---|
| MLN 1202 | Millennium | atherosclerosis |
| | Pharmaceuticals | (see also autoimmune) |
| | Cambridge, MA | |
| pexelizumab | Alexion | acute myocardial |
| | Pharmaceuticals | infarction, cardiopulmonary |
| | Cheshire, CT | bypass |
| | Procter & Gamble | |
| | Pharmaceuticals | |
| | Mason, OH | |

Diabetes and Related Conditions

| | | |
|---|---|---|
| anti-CD3 MAb | MacroGenics Rockville, | type-1 diabetes mellitus |
| | MD | |
| OKT3-gamma-1 | Johnson & Johnson | type-1 diabetes mellitus |
| | Pharmaceutical | |
| | Research | |
| | & Development | |
| TRX 4 | TolerRx | type-1 diabetes mellitus |
| (anti-CD3) | Cambridge, MA | |

Digestive Disorders

| | | |
|---|---|---|
| Remicade ™ | Centocor | Crohn's disease, |
| infliximab | | |
| ABT 874 | Abbott Laboratories | Crohn's disease |
| | Abbott Park, IL | (see also autoimmune) |
| CNTO 1275 | Centocor | Crohn's disease Phase II |
| | Horsham, PA | (see also autoimmune, skin) (610) 651-6000 |
| Humira ® | Abbott Laboratories | Crohn's disease Phase III |
| adalimumab | Abbott Park, IL | (see also autoimmune, skin) (847) 936-1189 |
| MDX-066 | Medarex | C. difficile disease |
| (CDA-1) | Princeton, NJ | |
| MDX-1100 | Medarex | ulcerative colitis |
| | Princeton, NJ | |
| MLN-02 | Millennium | ulcerative colitis |
| | Pharmaceuticals | |
| | Cambridge, MA | |
| Nuvion ® | PDL BioPharma | I.V. steroid-refractory |
| visilizumab | Fremont, CA | ulcerative colitis |
| | | Crohn's disease |
| Tysabri ® | Biogen Idec | Crohn's disease |
| natalizumab | Cambridge, MA | |

Eye Conditions

| | | |
|---|---|---|
| golimumab | Centocor Horsham, PA | uveitis (see also autoimmune) |

Genetic Disorders

| | | |
|---|---|---|
| Soliris ™ | Alexion Pharmaceuticals | paroxysmal nocturnal |
| eculizumab | Cheshire, CT | hemoglobinuria (PNH) |
| (Orphan Drug) | | |

Neurological Disorders

| | | |
|---|---|---|
| RN624 | Rinat Neuroscience | osteoarthritis pain |
| | South San Francisco, CA | |
| RN1219 | Rinat Neuroscience | Alzheimer's disease |
| | South San Francisco, CA | |

Respiratory Disorders

| | | |
|---|---|---|
| ABN 912 | Novartis Pharmaceuticals | asthma, chronic |
| | East Hanover, NJ | obstructive pulmonary |
| | | disorders (COPD) |
| ABX-IL8 | Amgen | COPD |
| | Thousand Oaks, CA | |
| AMG 317 | Amgen | asthma |
| | Thousand Oaks, CA | |
| daclizumab | Protein Design Labs | asthma |
| (anti-CD25 | Fremont, CA Roche | (see also autoimmune) |
| MAb) | Nutley, NJ | |
| MEDI-528 | MedImmune | asthma |
| anti-IL-9 MAb | Gaithersburg, MD | |
| mepolizumab | GlaxoSmithKline | asthma and nasal polyposis |
| (anti-IL5 MAb) | Philadelphia, PA | (see also other) |
| | Rsch. Triangle Park, NC | |
| TNX-832 | Tanox | respiratory diseases |
| | Houston, TX | |
| Xolair ® | Genentech | pediatric asthma |
| omalizumab | South San Francisco, CA | (see also other) |
| | Novartis Pharmaceuticals | |

Skin Disorders

| | | |
|---|---|---|
| Raptiva ® | Genentech | chronic moderate to severe plaque psoriasis |
| efalizumab | XOMA | |
| CNTO 1275 | Centocor | psoriasis |
| | | see also autoimmune, |
| | | digestive) |
| Humira ® | Abbott Laboratories | psoriasis |
| adalimumab | | see also autoimmune, |
| | | digestive) |
| TRX 4 | TolerRx | psoriasis |
| | | (see also diabetes) |

Transplatation

| | | |
|---|---|---|
| ORTHOCLONE | Ortho Biotech | acute kidney transplant rejection, reversal of heart |
| OKT ® 3 | | and liver transplant |
| muromonab- | | rejection |
| CD3 | | |
| Simulect ® | Novartis | prevention of renal transplant rejection |
| basiliximab | Pharmaceuticals | |
| Zenapax ® | Roche | prophylaxis of acute kidney transplant rejection |
| daclizumab | Protein Design Labs | |
| OKT3-gamma-1 | Johnson & Johnson | renal transplant rejection |
| | | (see also autoimmune, diabetes) |

Other

| | | |
|---|---|---|
| NeutroSpec ™ | Palatin Technologies | diagnosis of appendicitis |
| technetium | | |
| 99m | | |
| Tc | | |
| fanolesomab | | |
| CR 0002 | CuraGen | kidney inflammation |
| denosumab | Amgen | Postmenopausal osteoporosis, see also |
| (AMG 162) | | autoimmune and cancer |
| mepolizumab | GlaxoSmithKline | hypereosinophilic |
| (anti-IL5 MAb) | | syndrome, eosinophilic |
| | | esophagitis (see also respiratory) |
| Xolair ® | Genentech | peanut allergy(see also respiratory) |
| omalizumab | Tanox | |

Interferon Biopharmaceuticals

| | | |
|---|---|---|
| Actimmune ® | InterMune | management of chronic granulomatous disease |
| interferon | | malignant osteopetrosis |
| gamma-1b | | |
| Alferon N | Hemispherx | genital warts |
| Injection ® | Biopharma | |
| Interferon alfa-n2 | | |
| Avonex ® | Biogen Idec | relapsing multiple sclerosis |
| interferon beta-1a | | treatment after initial multiple sclerosis attack if |
| | | brain MRI shows abnormalities characteristic |
| | | of the disease |
| Betaseron ® | Berlex Laboratories | relapsing-remitting multiple sclerosis |
| recombinant | | relapsing forms of multiple sclerosis |
| interferon beta-1b | | |
| Infergen ® | Amgen | treatment of chronic hepatitis C viral infection |
| interferon alfacon-1 | | |
| Intron ® A | Schering-Plough | hairy cell leukemia |
| interferon alfa-2b | | genital warts |
| (recombinant) | | AIDS-related Kaposi's sarcoma |
| | | hepatitis C |
| | | hepatitis B |
| | | malignant melanoma |
| | | follicular lymphoma |
| | | hepatitis B in pediatric patients |
| Pegasys ® | Roche | chronic hepatitis C |
| peginterferon | | hepatitis B |
| alfa-2a | | |
| Rebetron ™ | Schering-Plough | treatment of chronic hepatitis C |
| ribavirin/interferon | | |
| alfa-2b, | | |
| combination | | |
| therapy | | |
| Rebif ® | Serono | relapsing forms of multiple sclerosis |
| interferon beta-1a | | |
| Roferon ®- | Roche | hairy cell leukemia |
| Ainterferon alfa- | | AIDS-related Kaposi's sarcoma |
| 2a, recombinant | | Chronic myelogenous leukemia |
| | | hepatitis C |
| Alferon N | Hemispherx | multiple sclerosis |
| Injection ® | Biopharma | |
| Avonex ® | Biogen | chronic inflammatory, demyelinating |
| interferon beta-1a | | polyneuropathy (CIDP) |
| interferon alpha | Amarillo Biosciences | Sjogren's syndrome, fibromyalgia, human |
| | | papillomavirus |
| Tauferon ™- | Pepgen | multiple sclerosis |
| interferon-tau | | |
| Veldona ® | Amarillo Biosciences | Behcet's disease, essential thrombocythemia |
| Lozenges - | | and polycythemia vera |
| natural human | | |
| interferon alpha | | |
| BLX 883 | Biolex | Cancer, hepatitis C treatment |
| interferon alpha- | Aradigm | Cancer, hepatitis C treatment |
| 2b - inhalation | | |
| PEG-Intron ® | Schering-Plough | malignant melanoma |
| peginterferon | | |
| alfa-2b | | |
| Albuferon ™ | Human Genome | chronic hepatitis C |
| albumin-interferon | Sciences | |
| alpha | | |
| Alferon LDO ® | Hemispherx | antiviral indications |
| | Biopharma | |
| Alferon N | Hemispherx | West Nile Virus |
| interferon | Biopharma | |
| Injection ® | | |
| Alpha-n3 | | |
| interferon omega | Intarcia Therapeutics | hepatitis C |
| Locteron ™ | Biolex Therapeutics | hepatitis C |
| controlled-release | | |
| alfa interferon | | |

### Kit of Parts:

As described above one aspect of the present invention relates to a kit-of-parts suitable for practising the methods according to the invention.

Accordingly the kit should comprise the reagents necessary for obtaining the genomic genetic code for at least one polymorphic locus in at least one PRR-gene, such as a PRR gene chosen from the group consisting of TLR1, TLR2, TLR3, TLR4, TLR5, TLR6, TLR7, TLR8, TLR9, TLR10, IFIH1 (MDA5), DDX58 (RIG-I), NOD1 (CARD4), and NOD2 (CARD15).

Optionally the kit may further comprise a control sample, such as DNA-samples with known genotypes for the at least one polymorphic locus.

Optionally the kit may further comprise instructions for use, such as described in the examples.

Optionally the kit may further comprise a piece of software capable of performing the genotype calls based on MFI-values, such as described in the section “Genotype Calls”.

The kit may comprise at least one PCR-primer set, such as at least one primer set as provided in table 4.

The kit may comprise at least one PCR-primer set such as a polynucleotide comprising a nucleotide sequence corresponding to any one sequence of SEQ ID NO: 1-252.

The kit may comprise at least one ASPE-primer set, such as a primer set as provided in table 5, such as the ASPE-primer sets corresponding anti-tag coupled bead-set, such as a FlexMAP® bead-set, such as provided in table 5, such as primer sets described in the examples section, such as a polynucleotide comprising a nucleotide sequence corresponding to any one sequence of SEQ ID NO: 117-252.

The PCR-primer set consisting of at least one forward and at least one reverse primer sequence, such as provided in table 4, such as a polynucleotide comprising a nucleotide sequence corresponding to any one sequence of SEQ ID NO: 1-116. The primer sequence being capable of mediating the amplification of a sequence of genetic material, such as DNA, containing at least one polymorphic locus, such as at least one SNP, when subjected to an appropriate PCR-thermocycling sequence and in combination with an appropriate PCR-reagent mixture.

In some embodiments the kit comprises at least one primer set according to the following table:

| | SEQ ID NOs for PCR- | SEQ ID NOs for ASPE- | SEQ ID NOs for |
|---|---|---|---|
| | primers | primers | FlexMAP bead-sets |
| TLR2.2 | 3 + 4, 39 + 40 | 119 + 120, 171 + 172 | 18 + 41 |
| TLR5.3 | 11 + 12, 55 + 56 | 133 + 134, 187 + 188 | 21 + 22 |
| TLR6.3 | 65 + 66 | 197 + 198 | 69 + 93 |
| TLR7.1 | 17 + 18, 67 + 68 | 145 + 146, 199 + 200 | 65 + 47 |
| TLR8.1 | 25 + 26, 75 + 76 | 153 + 154, 207 + 208 | 90 + 40 |
| TLR9.1 | 13 + 14, 81 + 82 | 139 + 140, 215 + 216 | 64 + 82 |
| TLR10.4 + 5 | 89 + 90, 91 + 92 | 225 + 226, 227 + 228 | 99 + 83, 11 + 52 |
| CARD4.1 + 2 + 3 | 103 + 104, 105 + 106, | 239 + 240, 241 + 242, | 5 + 99, 82 + 28, 87 + 10 |
| | 107 + 108 | 243 + 244 | |
| CARD15.4 | 115 + 116 | 251 + 252 | 18 + 60 |

The kit may further comprise a PCR-reagent mixture. The PCR-reagent mixture preferably comprise of at least a polymerase, such as a thermophilic polymerase, such as a temporarily inactivated thermophilic polymerase capable of regaining its activity if exposed to an appropriate thermocycling programme or activation step, such as described in the example section.

The kit or PCR-reagent mixture may further comprise the necessary molecular building blocks for creating a genetic sequence, such as at least the nucleotides deoxyadenosine-triphosphate (dATP), deoxyguanosine-triphosphate (dGTP), deoxycytidine-triphosphate (dCTP), and deoxythymidine-triphosphate (dTTP), such as at least the nucleotides dATP, dGTP, dCTP, and deoxyuridine-triphosphate (dUTP), such as at least the nucleotides dATP, dGTP, dCTP, dTTP, and dUTP, or corresponding nucleic acid analogues such as locked nucleic acids (LNA)®, or any combination thereof, and appropriate PCR-buffer salts and PCR-reaction enhancing additives, such as described in the examples section,

The kit or PCR-reagent mixture may further comprise water such as deionized or distilled water, such as DEPC-treated water, such as sterile filtered water, or any combination thereof.

The ASPE-primer set consists of at least two ASPE-primer sequences comprising an allele-specific nucleotide in any one end of the sequence, such as in the 3′-end, and a capture sequence, such as a FlexMAP® tag-sequence, in the opposite end, such as the 5′-end, and joined by a nucleotide sequence, capable of adhering to the sequence immediately next to the polymorphic locus.

The ASPE-primer sequences should be capable of being elongated by a polymerase if a nucleotide or nucleotide analogue that is complementary to the allele-specific nucleotide is present in the polymorphic locus, when subjected to an appropriate ASPE-thermocycling sequence and in combination with an appropriate ASPE-reagent mixture.

The ASPE-reagent mixture may consist of at least a polymerase, such as a thermophilic polymerase, such as a temporarily inactivated thermophilic polymerase capable of regaining its activity if exposed to an appropriate thermocycling programme or activation step, and at least one labelled nucleotide or nucleotide analogue, such as biotinylated-dCTP, such as biotinylated-dUTP, such as biotinylated-dATP, such as biotinylated-dGTP, such as biotinylated-dTTP, or any combination thereof, such as biotinylated-dCTP in combination with biotinylated-dUTP, such as described in the examples section.

In a preferred embodiment, the kit is suitable for performing an allele-specific primer extension (ASPE)-based assay, such as described in the example section.

In some embodiments, the kit comprises a PCR-reagent mixture corresponding to the commercially available Qiagen Multiplex PCR Kit, such as Qiagen catalog number: 206143, such as Qiagen catalog number: 206145.

In some embodiments, the kit comprises an ASPE-reagent mixture corresponding to the commercially available Platinum® Genotype Tsp Polymerase Kit, such as Invitrogen catalog number: 11448-024, such as Invitrogen catalog number: 11448-032.

In some embodiments, the anti-tag coupled bead-sets constitute FlexMAP® bead-sets.

In some embodiments, the kit comprises the reagents necessary for genotyping at least one polymorphic locus in at least one PRR-gene, such as at least two polymorphic loci in at least one PRR-gene, such as at least three polymorphic loci in at least one PRR-gene, such as at least five polymorphic loci in at least one PRR-gene, such as at least ten polymorphic loci in at least one PRR-gene.

In some embodiments, the kit comprises the reagents necessary for genotyping at least two polymorphic loci in at least two PRR-genes, such as at least two polymorphic loci in at least two PRR-genes, such as at least three polymorphic loci in at least three PRR-genes, such as at least four polymorphic loci in at least four PRR-genes, such as at least four polymorphic loci in at least three PRR-genes.

In some embodiments, the kit comprises at least one PCR-primer set provided in table 4, and at least one corresponding ASPE-primer set provided in table 5, and at least one FlexMAP® bead-set provided in table 5.

In some embodiments, the kit further comprises the biopharmaceutical according to the biopharmaceutical treatment.

In some embodiments, the kit further comprises means for performing the methods of the invention, such as PCR tubes and plates, package inserts with instructions for use, and data carriers containing software capable of performing the data analysis according to the methods of the invention.

### REFERENCES

1. Bendtzen K, Marker O, Svehag S-E, Svejgaard A, Kilian M. Basal og klinisk Immunologi. København: FADL's Forlag, 2000
2. Carroll M C, Janeway C A. Innate immunity. Curr Opin Immunol 1999; 11:11-2.
3. Janeway C A, Jr. How the immune system works to protect the host from infection: A personal view. Proc Natl Acad Sci USA 2001; 5:1-8.
4. Medzhitov R, Janeway C, Jr. The Toll receptor family and microbial recognition. Trends Microbiol 2000; 8:452-6.
5. Bendtzen K, Nielsen H, Petersen J. Treatment of rheumatoid arthritis with anti-TNF antibodies [Article in Danish]. Ugeskr Laeger 1995; 157:1689-90.
6. Cook D N, Pisetsky D S, Schwartz D A. Toll-like receptors in the pathogenesis of human disease. Nat Immunol 2004; 5:975-9.
7. Bendtzen K, Bovin L F, Andersen V, Rieneck K, Svenson M. Toll-like receptors. What can we learn about the immune system from banana flies and a mouse strain [Article in Danish]. Ugeskr Laeger 2001; 163:4562-5.
8. Akira S, Takeda K. Toll-like receptor signalling. Nature Rev Immunol 2004; 4:499-511.
9. Creagh E M, O'Neill L A. TLRs, NLRs and RLRs: a trinity of pathogen sensors that co-operate in innate immunity. Trends Immunol 2006; 27:352-7.
10. O'Neill L A. Targeting signal transduction as a strategy to treat inflammatory diseases. Nat Rev Drug Discov 2006; 5:549-63.
11. Marshak-Rothstein A. Toll-like receptors in systemic autoimmune disease. Nat Rev Immunol 2006; 6:823-35.
12. Matzinger P. Friendly and dangerous signals: is the tissue in control? Nat Immunol 2007; 8:11-3.
13. Feldmann M, Brennan F M, Foxwell B M, Taylor P C, Williams R O, Maini R N. Anti-TNF therapy: where have we got to in 2005? J Autoimmun 2005; 25 Suppl:26-8.
14. Jarvis B, Faulds D. Etanercept: a review of its use in rheumatoid arthritis. Drugs 1999; 57:945-66.
15. Genovese M C, Kremer J M. Treatment of rheumatoid arthritis with etanercept. Rheum Dis Clin North Am 2004; 30:311-28, vi-vii.
16. Klareskog L, van der Heijde D, de Jager J P, Gough A, Kalden J, Malaise M, Martin Mola E, Pavelka K, Sany J, Settas L, Wajdula J, Pedersen R, Fatenejad S, Sanda M. Therapeutic effect of the combination of etanercept and methotrexate compared with each treatment alone in patients with rheumatoid arthritis: double-blind randomised controlled trial. Lancet 2004; 363:675-81.
17. Feldmann M, Elliott M J, Woody J N, Maini R N. Anti-tumor necrosis factor-a therapy of rheumatoid arthritis. Adv Immunol 1997; 64:283-350.
18. Moreland L W. Drugs that block tumour necrosis factor: experience in patients with rheumatoid arthritis. Pharmacoeconomics 2004; 22:39-53.
19. Weisman M H, Moreland L W, Furst D E, Weinblatt M E, Keystone E C, Paulus H E, Teoh L S, Velagapudi R B, Noertersheuser P A, Granneman G R, Fischkoff S A, Chartash E K. Efficacy, pharmacokinetic, and safety assessment of adalimumab, a fully human anti-tumor necrosis factor-alpha monoclonal antibody, in adults with rheumatoid arthritis receiving concomitant methotrexate: a pilot study. Clin Ther 2003; 25:1700-21.
20. Scheinfeld N. Adalimumab (HUMIRA): a review. J Drugs Dermatol 2003; 2:375-7.
21. Bendtzen K. Adalimumab in the treatment of rheumatoid arthritis [Article in Danish]. Ugeskr Laeger 2004; 166:2552-4.
22. Bender N K, Heilig C E, Droll B, Wohlgemuth J, Armbruster F P, Heilig B. Immunogenicity, efficacy and adverse events of adalimumab in RA patients. Rheumatol Int 2006;
23. Wolbink G J, Voskuyl A E, Lems W F, de Groot E, Nurmohamed M T, Tak P P, Dijkmans B A, Aarden L. Relationship between serum trough infliximab levels, pretreatment C reactive protein levels, and clinical response to infliximab treatment in patients with rheumatoid arthritis. Ann Rheum Dis 2005; 64:704-7.
24. Wolbink G J, Vis M, Lems W, Voskuyl A E, de Groot E, Nurmohamed M T, Stapel S, Tak P P, Aarden L, Dijkmans B. Development of antiinfliximab antibodies and relationship to clinical response in patients with rheumatoid arthritis. Arthritis Rheum 2006; 54:711-5.
25. Bendtzen K, Geborek P, Svenson M, Larsson L, Kapetanovic M C, Saxne T. Individualized monitoring of drug bioavailability and immunogenicity in rheumatoid arthritis patients treated with the tumor necrosis factor alpha inhibitor Infliximab. Arthritis Rheum 2006; 54:3782-9.
26. Russell E, Zeihen M, Wergin S, Litton T. Patients receiving etanercept may develop antibodies that interfere with monoclonal antibody laboratory assays. Arthritis Rheum 2000; 43:944.
27. Gudbrandsdottir S, Bliddal H, Petri A, Terslev L, Danneskiold-Samsoe B, Bjørnhart B, Bendtzen K, Müller K. Plasma TNF binding capacity profiles during treatment with etanercept in rheumatoid arthritis. Scand J Rheumatol 2004; 33:385-8.
28. Anderson P J. Tumor necrosis factor inhibitors: Clinical implications of their different immunogenicity profiles. Semin Arthritis Rheum 2005; 34:19-22.
29. Bartelds G M, Wolbink G J, Stapel S, Aarden L, Lems W F, Dijkmans B A, Nurmohamed M T. High levels of human anti-human antibodies to adalimumab in a patient not responding to adalimumab treatment.[letter]. Ann Rheum Dis 2006; 65(9):1249-50.
30. Schellekens H. Factors influencing the immunogenicity of therapeutic proteins. Nephrol Dial Transplant 2005; 20 Suppl 6:vi3-9.
31. Blander J M. Coupling Toll-like receptor signaling with phagocytosis: potentiation of antigen presentation. Trends Immunol 2007; 28:19-25.
32. Palmer S M, Burch L H, Davis R D, Herczyk W F, Howell D N, Reinsmoen N L, Schwartz D A. The role of innate immunity in acute allograft rejection after lung transplantation. Am J Respir Crit Care Med 2003; 168:628-32.
33. Methe H, Zimmer E, Grimm C, Nabauer M, Koglin J. Evidence for a role of toll-like receptor 4 in development of chronic allograft rejection after cardiac transplantation. Transplantation 2004; 78:1324-31.
34. Ducloux D, Deschamps M, Yannaraki M, Ferrand C, Bamoulid 3, Saas P, Kazory A, Chalopin J M, Tiberghien P. Relevance of Toll-like receptor-4 polymorphisms in renal transplantation. Kidney Int 2005; 67:2454-61.
35. Rozen S, Skaletsky H. Primer3 on the WWW for general users and for biologist programmers. In: Krawetz S, Misener S, eds. Bioinformatics Methods and Protocols: Methods in Molecular Biology. Totowa, N.J., USA: Humana Press, 2000:365-86.
36. Ye F, Li M S, Taylor J D, Nguyen Q, Colton H M, Casey W M, Wagner M, Weiner M P, Chen J., Fluorescent microsphere-based readout technology for multiplexed human single nucleotide polymorphism analysis and bacterial identification., Hum Mutat. 2001 April; 17(4):305-16.
37. Taylor J D, Briley D, Nguyen Q, Long K, Iannone M A, Li M S, Ye F, Afshari A, Lai E, Wagner M, Chen J, Weiner M P., Flow cytometric platform for high-throughput single nucleotide polymorphism analysis., Biotechniques. 2001 March; 30(3):661-6, 668-9.
38. Chen J, Iannone M A, Li M S, Taylor J D, Rivers P, Nelsen A J, Slentz-Kesler K A, Roses A, Weiner M P., A microsphere-based assay for multiplexed single nucleotide polymorphism analysis using single base chain extension., Genome Res. 2000 April; 10(4):549-57.
39. Landegren U, Kaiser R, Sanders 3, Hood L., A ligase-mediated gene detection technique., Science. 1988 Aug. 26; 241(4869):1077-80.
40. Dunbar S A, Jacobson J W., Rapid screening for 31 mutations and polymorphisms in the cystic fibrosis transmembrane conductance regulator gene by Lminex xMAP suspension array., Methods Mol. Med. 2005; 114:147-71

### EXAMPLES

### Materials

Carboxylated fluorescent microspheres with covalently attached FlexMAP anti-TAG sequences (FlexMAP beads).
HPLC-purified PCR amplification primers for each target resuspended in sterile ddH₂O.
HPLC-purified ASPE primers with 5′ TAG modification resuspended in sterile ddH₂O.
Qiagen Multiplex PCR kit (Qiagen Cat. No. 206143)
Platinum Tsp, ASPE 10× Buffer, 50 mM MgCl₂ (Invitrogen Cat. No. 11448-024)
dNTPs at 100 mM each (Invitrogen Cat. No. 10297-018)
Biotin-14-dCTP at 0.4 mM (Invitrogen Cat. No. 19518-018)
Biotin-11-dUTP at 0.4 mM (Yorkshire Bioscience Ltd. Cat. No. P1611)
1.5×TMAC hybridization solution (see appendix A)
1×TMAC hybridization solution (see appendix A)
Streptavidin-R-phycoerythrin (ProZyme Cat. No. PJ331S)
96 well V-bottom PCR plate and cover, Pipette tips, disposable gloves, PCR-tubes, etc.
Genomic DNA samples

### Procedures

### Multiplexed PCR Reaction

### Each Final Reaction (10 uL) Contained:

1× Qiagen Multiplex PCR Mastermix
0.2 μM each primer
10 ng template

| | |
|---|---|
| 2X Qiagen Multiplex PCR | 5.0 μL |
| Mastermix | |
| Genomic Template (10 ng/μL) | 1.0 μL |
| PrimerMix (1 pmol/μL each | 2.0 μL |
| primer) | |
| dH₂O | 2.0 μL |
| | 10.0 μL |

### PCR Cycling Parameters:

| | |
|---|---|
| HOLD: | 95° C., 15 minutes (for enzyme activation) |
| CYCLE: | 94° C., 30 seconds |
| | 60° C., 3 minutes |
| | 72° C., 90 seconds |
| | 40 CYCLES |
| HOLD | 68° C., 15 minutes |
| HOLD | 4° C., FOREVER |

Samples were stored at ≦−18 C.° as soon as possible after having reached the final step of the PCR-cycle, until further use in the subsequent reactions.

### Multiplex ASPE Reaction:

### Each 5 μL a Final Reaction Contained:

1×ASPE Buffer (20 mM Tris-HCl, pH 8.4; 50 mM KCl)
1.25 mM MgCl₂
25 nM each TAG-ASPE primer
0.375 U Tsp DNA polymerase
5 μM dATP, dGTP
5 μM biotin-dCTP, biotin-dUTP
0.2 μL PCR reaction
dH₂O to 5 μL

We have found that using multiple, such as two labels, such as two radio labels, in the ASPE reaction step it is possible to increase the number of SNPs which can be detected in a single multiplex reaction.

### ASPE Reaction Mix (5 μL/Reaction):

| | | |
|---|---|---|
| 10X ASPE reaction buffer | 0.5 | μL |
| 50 mM MgCl₂ | 0.125 | μL |
| 20X TAG-ASPE primer mix | 0.25 | μL (diluted 100 μM (=100 pmol/μL) stocks 1:200 for 20X mix) |
| (500 nM each) | | |
| 5 U/μL Tsp DNA polymerase | 0.075 | μL |
| 20X dNTP mix (dATP and | 0.25 | μL (diluted 100 mM stocks 1:1000 for 20X mix) |
| dGTP) (100 μM each) | | |
| 400 μM biotin-dCTP | 0.0625 | μL (0.4 mM, 50 nmol, 125 μL per tube) |
| 400 μM biotin-dUTP | 0.0625 | μL (10 mM, 500 nmol, 50 μL per tube - dilute 1:25) |
| Qiagen Multiplex PCR-product | 0.2 | μL |
| dH₂O | 3.4875 | μL |
| | 5.0 | μL |

### ASPE Cycling Parameters:

| | |
|---|---|
| HOLD: | 96° C., 2 minutes |
| CYCLE: | 94° C., 30 seconds |
| | 50° C., 1 minute |
| | 74° C., 2 minutes |
| | 30 CYCLES |
| HOLD | 4° C., FOREVER |

Samples were stored at ≦−18 C.° as soon as possible after having reached the final step of the PCR-cycle, until further use in subsequent reactions.

### Hybridization to FlexMAP Microspheres:

(Microspheres were protected from prolonged exposure to light throughout this procedure.)

The following steps were completed in order to achieve this objective:
1. The appropriate FlexMAP microsphere sets were selected and resuspend by vortex and sonicated for approximately 30 seconds.
2. 250 microspheres of each set were combined per reaction (1 μL of each selected beadset per reaction) (A surplus of 1.5×TMAC buffer was added).
3. The FlexMAP microsphere mixture was concentrated by centrifugation at ≧8000×g for 3 minutes.
4. The supernatant was removed and resuspended to 250 of each microsphere set per 45 μL (45 μL per reaction) in 33 μL 1.5×TMAC Hybridization Buffer and 12 μL H₂O by vortex and sonication for approximately 20 seconds.
5. Aliquoted 45 μL of the FlexMAP microsphere mixture to each well.
6. Added 5 μL of each ASPE reaction to appropriate wells.
7. (Adjusted the total volume to 50 μL by adding the appropriate volume of dH₂O to each sample well, where necessary.)
8. Covered the plate to prevent evaporation and denature at 96° C. for 5 minutes.
9. Hybridized at 37° C. for 60 minutes.
10. Pelleted the FlexMAP microspheres by centrifugation at ≧2400× rcf for 3 minutes and removed the supernatant.
11. Resuspended the pelleted FlexMAP microspheres in 100 μL of 1×SSPET Stringent Wash Buffer.
12. Pelleted the FlexMAP microspheres by centrifugation at 2400× rcf for 3 minutes and removed the supernatant.
13. Resuspended microspheres in 70 μL of 1× TMAC Hybridization Buffer containing 8 μg/mL streptavidin-R-phycoerythrin.
14. Incubated at 4° C. (approx.) overnight, typically for between 16 and 24 hours. Analyze 70 μL at 37° C. on the Luminex100 analyzer according to the system manual.

### APPENDIX A

20% Sarkosyl 250 mL

| | | Final | Amount/ |
|---|---|---|---|
| Reagent | Catalog Number | Concentration | 250 mL |
| Sarkosyl | Sigma L-9150 | 20% | 50 g |
| (N-Lauroylsarcosine) | | | |

Filter Sterilized and stored at Room Temperature

1.5 X TMAC Hybridization Solution
(MICROSPHERE DILUENT) 250 mL

| | | Final | Amount/ |
|---|---|---|---|
| Reagent | Catalog Number | Concentration | 250 mL |
| 5 M TMAC | Sigma T-3411 | 4.5 M | 225 mL |
| 20% Sarkosyl | — | 0.15% | 1.88 mL |
| 1 M Tris-HCl, pH 8.0 | Sigma T-3038 | 75 mM | 18.75 mL |
| 0.5 M EDTA pH 8.0 | Invitrogen 15575- | 6 mM | 3.0 mL |
| | 020 | | |
| H₂O | — | — | 1.37 mL |

Stored at Room Temperature

1 X TMAC Hybridization Solution (DETECTION BUFFER) 250 mL

| | | Final | Amount/ |
|---|---|---|---|
| Reagent | Catalog Number | Concentration | 250 mL |
| 5 M TMAC | Sigma T-3411 | 3 M | 150 mL |
| 20% Sarkosyl | — | 0.1% | 1.25 mL |
| 1 M Tris-HCl, pH 8.0 | Sigma T-3038 | 50 mM | 12.5 mL |
| 0.5 M EDTA pH 8.0 | Invitrogen 15575- | 4 mM | 2 mL |
| | 020 | | |
| H₂O | — | — | 84.25 mL |

Stored at Room Temperature

6X SSPET (STRINGENT WASH BUFFER) 250 mL

| | | Final | Amount/ | |
|---|---|---|---|---|
| Reagent | Catalog Number | Concentration | 250 mL | |
| SSPE (20X | Sigma S-2015 | 6X concentrate | 75 | mL |
| concentrate) - Saline, | | | | |
| Sodium Phosphate, | | | | |
| EDTA | | | | |
| Triton X-100 | Sigma T-9284 | 0.005%-0.01% | 12.5-25 | μL |

Filter Sterilized and stored at Room Temperature

### Selection of Single Nucleotide Polymorphisms (SNPs)

The SNPs selected for the assays were primarily SNPs causing non-conservative amino-acid substitutions but also SNPs in promoter regions, 3′-untranslated regions (UTR), exons and exon/intron boundary regions were included (table 3). All SNPs were selected based on informations available at the dbSNP (http://www.ncbi.nlm.nih.gov/SNP/), SNPper (http://snpper.chip.org/bio/) and IIPGA (http://www.innateimmunity.net/) databases. Only bi-allelic SNPs that were found in persons of Caucasian descent, with a heterozygote frequency of at least 1%, according to previous findings in the above-mentioned databases, were included in the assays.

### Assay Description

Four assays were developed capable of analyzing 13 SNPs located in the human TLR2, 4, 5 and 9 genes-9 SNPs located in the human TLR 3, 7 and 8 genes-34 SNPs located in the TLR1 through 10 genes and 11 SNPs located in the human MDA5, DDX58, CARD4 and CARD15 genes respectively.

SNPs were determined in a multiplexed fashion, using flow cytometric, bead-based assays and a Luminex 100IS flow cytometer (Luminex Corporation, Austin, Tex., USA). These assays were comprised of 4 consecutive multiplexed steps:
1. A multiplexed polymerase chain reaction (PCR) in which the SNP sites of interest were amplified.
2. An allele-specific primer extension reaction (ASPE) in which biotinylated nucleotides (biotin-dCTP, and -dUTP) was incorporated into ASPE-primers matching the genotype of the sample.
3. Sorting on the FlexMAP bead-array by hybridization.
4. Detection using the Luminex 100IS flow cytometer.

The FlexMAP-bead array consists of a predefined set of 100 fluorescently labeled polystyrene microbeads with a diameter of 5.6 μM, which are well suited for the capture, and analysis of ASPE-primers in a multiplexed fashion. Each of the FlexMAP-beadsets is coupled to an “anti-tag” sequence, a 24-mer oligonucleotide complementary to a “tag” sequence that is used to identify the individual ASPE-primers. Each ASPE-primer is constructed so the 3′-end defines the SNP site, with the 3′-end nucleotide overlapping the polymorphic site, while the 5′-end is composed of a tag-sequence enabling easy sorting of the up to 100 different tagged primers, in a single reaction tube. For each existing allele at each polymorphic site, one ASPE-primer was constructed. The alleles present in the sample form perfect hybridizations with their respective ASPE-primers including the 3′-end of the primer, enabling the polymerase to elongate the primers incorporating biotinylated dCTP and -dUTP, while the ASPE-primers of alleles not present in the sample, will not form perfect 3′-end hybridizations and consequently will not be elongated by the polymerase. In this way ASPE-primers corresponding to alleles present in the sample are biotinylated, while ASPE-primers corresponding to alleles not present in the sample are not. The ensuing hybridization to FlexMAP-beads and incubation with streptavidin-phycoerythrin (SA-PE) reporter then enabled easy identification of each ASPE-primer and whether or not the corresponding allele was present in the sample by assessment of the median fluorescence intensity (MFI) associated with each bead set.

All PCR- and ASPE reactions as well as bead-hybridizations were performed in 96-well, 0.2 mL PCR-plates on either an Opticon 2 thermal cycler (MJ Research, Waltham, Mass., USA) or a GeneAmp PCR System 9600 (Perkin Elmer Corporation, Wellesley, Mass., USA).

### Multiplex PCR

The primer sequences for the multiplex PCR-reactions were designed using Primer3 [35], producing primers of 19-22 nucleotides (table 4).

Multiplexed PCR reactions were performed using Qiagen Multiplex Mastermix (Qiagen GmbH, Hilden, Germany) following the guidelines provided by the manufacturer except for the fact that we used 10 μL of combined reaction mixture instead of 50 μL as suggested by the manufacturer. The specific multiplex PCR conditions, such as annealing temperature and time, number of cycles etc. were established in a series of preliminary experiments (data not shown). Each PCR reaction contained 1× Qiagen Multiplex Mastermix, 0.2 μM of each HPLC-purified PCR-primer (TAG Copenhagen A/S, Copenhagen, Denmark), 3 mM MgCl₂ and 10 ng genomic DNA in a total reaction volume of 10 μL. All primers where added at equimolar concentrations. The reactions were held at 95° C. for 15 min. to activate the polymerase, followed by 40 cycles at 94° C. for 30 sec., 60° C. for 3 min. and 72° C. for 90 sec. After a final extension at 68° C. for 15 min., the reactions were cooled to 4° C. and where then stored at ≦−18 C.° until use in the ASPE reactions.

### Multiplex ASPE

Since the ASPE-primers 3′-ends has to overlap the SNP-sites being questioned, these primers were designed manually, using their 3′-end as a fix-point and Primer3 (Rozen et al., 2000) to assess their corresponding melting temperatures, producing primers of 14-25 nucleotides with predicted melting temperatures ranging from 47.0 to 58.5 degrees Celsius (table 5). In some cases modifications of the primers were necessary due to the presence of additional SNPs in the primer sequence, or unintended cross reactivity with other primers or PCR-products, etc. In these cases the primer sequence was ether shortened appropriately or a new primer was created for the complementary allele sequence.

Subsequently all ASPE-primer sequences were ‘tagged’, i.e. each ASPE-primer sequence was appended to one of the 100 possible tags in the FlexMAP array, using Tag-IT software from TM Bioscience (Toronto, Ontario, USA), bringing the ASPE-primers to final lengths of 38 to 49 nucleotides.

Each ASPE reaction contained 0.375 U Platinum Genotype Tsp Polymerase, 20 mM Tris-HCl (pH 8.4), 50 mM KCl, 1.25 mM MgCl₂, 5 μM dATP, dTTP, dGTP and 5 μM biotin-dCTP (Invitrogen Corporation, Carlsbad, Calif., USA), 25 nM each HPLC-purified ASPE-primer (TAG Copenhagen A/S, Copenhagen, Denmark) and 0.2 μL PCR-product in a total reaction volume of 5 μL. The reactions were held at 96° C. for 2 min. to activate the polymerase, followed by 30 cycles at 94° C. for 30 sec., 50° C. for 1 min. and 74° C. for 2 min. finally the reactions were cooled to 4° C. and where then stored at ≦−18 C.° until sorting by hybridization to FlexMAP microspheres.

### FlexMAP Array Sorting and Detection on the Luminex 100IS XYP Platform.

For hybridization reactions approximately 250 of each of the appropriate anti-tag-coupled FlexMAP microspheres (Luminex Corporation, Austin, Tex., USA) were mixed, isolated by centrifugation and resuspended in 1.1× tetramethylammonium chloride (TMAC) buffer (3.3 M TMAC, 0.11% sarkosyl, 55 mM Tris-HCl, 4.4 mM EDTA) (Sigma-Aldrich, St. Louis, Mo., USA). 45 μL of this microsphere suspension were added to 5 μL of ASPE-product and the samples were hybridized by heating them to 96° C. for 2 min., followed by 37° C. for 60 min. The microspheres were then washed once in 100 μL of refrigerator-cold 1×SSPET (0.2 M phosphate buffer, pH 7.4, 2.98 M NaCl, 0.02 M EDTA, 0.01% Triton X-100) (Sigma-Aldrich, St. Louis, Mo., USA), resuspended in 70 μL of reporter solution containing 8 mg/mL SA-PE (ProZyme, San Leandro, Calif., USA) in 1.0×TMAC buffer, and incubated at 4° C. (approx.) overnight, typically for between 16 and 24 hours before being analyzed on the Luminex 100IS.

### Positive and Negative Controls and Random Re-Runs

To serve as positive controls with known genotypes, DNA from seven different individuals were purchased from the Coriell Cell Repository (CCR) at the Coriell Institute for Medical Research (Camden, N.J., USA), additionally DNA from four different individuals with known genotypes working at the Danish National University Hospital also served as assay controls (table 6). DNA from hospital employees was genotyped by sequencing at MWG-Biotech AG (Ebersberg, Germany). The positive controls were included in each assay run. A no-template PCR negative control was included in each assay run. Before further use of PCR-products, the no template negative control sample, along with one of the positive control samples and one of the samples to be genotyped, were analyzed on a Cambrex Flashgel (Rockland, N.Y., USA) (data not shown) to verify the production of PCR-products in the positive control sample and sample to be genotyped, while if any visible bands appeared in the no template negative control sample the entire plate was assumed contaminated and consequently discarded.

Likewise, an ASPE-reaction performed on the no-template PCR negative control was included in each assay run as a negative control.

After each plate run, at least five randomly chosen samples were re-typed on a new plate to verify the genotypes obtained in the first run.

### Genotype Calls

The genotype calls were performed for each SNP on each plate individually, using the MFI-values of the positive controls as a guideline for setting thresholds (FIG. 1). For each SNP the ratios of the MFIs for the two alleles were first calculated as: Allele A ratio=MFI_{allele A}/(MFI_{allele A}+MFI_{allele B}). Allelic ratios were then plotted in graphs (FIG. 1) and inspected visually to determine threshold settings for each SNP in each plate run. If the allelic ratio of one of the two alleles was high (usually above 0.80) the sample was considered a homozygote for that allele, and vice versa. If the allelic ratios of the two alleles were approximately equal (usually 0.30 to 0.70), the sample was considered a heterozygote.

If one or more of the SNP genotype calls for a given sample failed, the genotypes for the entire sample were discarded and the sample was run again. If one or more SNP genotype calls for a sample failed on three separate occasions, the sample was discarded and excluded from further analysis.

### Results

Respondership to TNF-a Blockade is Associated with SNPs in TLR7, TLR8 and TLR9 and is Differently Distributed in Infliximab Compared with Adalimumab.

Emerging evidence supports the role of Toll-like receptors (TLR) both in the initiation of the innate immune response as well as the tuning of the adaptive immune response. Since both innate and adaptive immune responses are thought to be important during rheumatoid arthritis, we here investigated whether SNPs in the various TLR subtypes are associated with respondership to the TNF-a neutralizing therapies Infliximab and Adalimumab. Among all tested SNPs, only one SNP located in the promoter region of TLR9 (TLR9.1) was clearly associated with responder ship to both Infliximab and Adalimumab. Whereas only 26.7% of the patients treated with Infliximab and who were classified as good responders carried the TLR9.1 C-allele, over 93% of the moderate responders and over 95% of the non-responders carried the TLR9.1 risk allele. Similarly, of the responders to Adalimumab, 17.6% of the patients were found to be heterozygous for the TLR9.1 C-allele whereas 5.9% were homozygous. In sharp contrast, 100% of the moderate responders and 90% of the non-responders carried at least one of the TLR9.1 risk alleles. For both the patients using Infliximab and Adalimumab, carriers of the TLR9.1 risk-allele were significantly more common in those who were classified as non-responders compared to those who only had a partial response to therapy.

Interestingly, SNPs in the TLR7 (TLR7.1) and TLR8 (TLR8.1) genes were associated with respondership to TNF-α only in those patients treated with Adalimumab. Of the patients that responded well to Adalimumab, 5.9% of the patients carried (only heterozygous patients) the TLR7.1 G-allele and 17.6% and 11.8% were found to carry one or two copies of the TLR8.1 T-allele, respectively. In contrast, 42.9% of the moderate responders and 70% of the non-responders carried at least one copy of the TLR7.1 risk allele. In line with this, 57.1% of the moderate responders and 70% of the non-responders carried at least one copy of the TLR8.1 risk allele. In contrast with that observed for the TLR9.1 SNP, the frequency of carrier ship of the TLR7.1 and TLR8.1 SNP was not significantly different among moderate versus non-responders.

### TLRs and Host Response to Protein Drugs

TABLE 3

Overview of investigated single nucleotide polymorphisms (SNPs)*
Overview of investigated nucleotide polymorphisms
(45 polymorphisms totally)

| | | | Major | Minor | Freq. | | Freq. | |
|---|---|---|---|---|---|---|---|---|
| PRR | rs# | Assay | allele | allele | Major | Heterozygous | Minor | Mutation |
| TLR1.1 | rs5743611 | C | G | C | 0.944 | 0.111 | 0.056 | Arg80Thr |
| TLR1.2 | rs4833095 | C | A | G | 0.800 | 0.300 | 0.200 | Asn248Ser |
| TLR1.3 | rs3923647 | C | A | T | 0.950 | 0.100 | 0.050 | His305Leu |
| TLR1.4 | rs5743618 | C | G | T | 0.750 | 0.400 | 0.250 | Ser602Ile |
| TLR2.1 | rs1898830 | A, C | A | G | 0.705 | 0.416 | 0.295 | Promoter |
| TLR2.2 | rs5743704 | A, C | C | A | 0.977 | 0.045 | 0.023 | Pro631His |
| TLR2.3 | rs5743708 | A, C | G | A | 0.975 | 0.050 | 0.025 | Arg753Gln |
| TLR3.1 | rs3775291 | B, C | G | A | 0.700 | 0.300 | 0.300 | Leu412Phe |
| TLR4.1 | rs7873784 | A, C | G | C | 0.826 | 0.287 | 0.174 | 3′UTR |
| TLR4.2 | rs4986790 | A, C | A | G | 0.957 | 0.083 | 0.043 | Asp299Gly |
| TLR4.3 | rs4986791 | A, C | C | T | 0.962 | 0.073 | 0.038 | Thr399Ile |
| TLR5.1 | rs764535 | A, C | G | A | 0.963 | 0.071 | 0.037 | Thr82Ile |
| TLR5.2 | rs5744168 | A, C | C | T | 0.935 | 0.122 | 0.065 | Arg392STOP |
| TLR5.3 | rs2072493 | A, C | A | G | 0.804 | 0.315 | 0.196 | Asn592Ser |
| TLR5.4 | rs5744174 | A, C | T | C | 0.652 | 0.454 | 0.348 | Phe616Leu |
| TLR5.5 | rs5744176 | A, C | A | G | 0.978 | 0.043 | 0.022 | Asp694Gly |
| TLR6.1 | rs5743815 | C | T | C | 0.950 | 0.100 | 0.050 | Val427Ala |
| TLR6.2 | rs5743813 | C | C | T | 0.992 | 0.017 | 0.008 | His345Tyr |
| TLR6.3 | rs5743810 | C | C | T | 0.500 | 0.500 | 0.500 | Ser249Pro |
| TLR7.1 | rs2302267 | B, C | T | G | 0.913 | 0.159 | 0.087 | Exon/intron |
| | | | | | | | | boundary |
| TLR7.2 | rs179008 | B, C | A | T | 0.783 | 0.340 | 0.217 | Gln11Leu |
| TLR7.3 | rs5743781 | B, C | C | T | 0.957 | 0.083 | 0.043 | Ala448Val |
| TLR7.4 | rs3853839 | B, C | C | G | 0.795 | 0.325 | 0.205 | 3′UTR |
| TLR8.1 | rs5741883 | B, C | C | T | 0.752 | 0.373 | 0.248 | Promoter |
| TLR8.2 | rs3764879 | B, C | C | G | 0.569 | 0.490 | 0.431 | Promoter |
| TLR8.3 | rs3764880 | B, C | A | G | 0.658 | 0.158 | 0.342 | Met1Val |
| | | | | | | | | (Startcodon) |
| TLR8.4 | rs5744088 | B, C | G | C | 0.755 | 0.370 | 0.245 | 3′UTR |
| TLR9.1 | rs187084 | A, C | T | C | 0.609 | 0.476 | 0.391 | Promoter |
| TLR9.2 | rs5743836 | A, C | T | C | 0.848 | 0.258 | 0.152 | Promoter |
| TLR10.1 | rs4129008 | C | G | A | 0.975 | 0.050 | 0.025 | Arg800Gln |
| TLR10.2 | rs4129009 | C | A | G | 0.850 | 0.300 | 0.150 | Ile775Val |
| TLR10.3 | rs11466657 | C | T | C | 0.950 | 0.095 | 0.100 | Ile473Thr |
| TLR10.4 | rs11096955 | C | A | C | 0.725 | 0.450 | 0.275 | Ile369Leu |
| TLR10.5 | rs11096957 | C | A | C | 0.725 | 0.450 | 0.275 | Asn241His |
| MDA5.1 | rs10930046 | D | A | G | 0.978 | 0.043 | 0.022 | His460Arg |
| MDA5.2 | rs3747517 | D | C | T | 0.727 | 0.364 | 0.273 | Arg843His |
| MDA5.3 | rs1990760 | D | T | C | 0.608 | 0.517 | 0.392 | Thr946Ala |
| DDX58.1 | rs10813831 | D | G | A | 0.686 | 0.356 | 0.314 | Arg7Cys |
| DDX58.2 | rs17217280 | D | A | T | 0.854 | 0.208 | 0.146 | Asp580Glu |
| CARD4.1 | rs2075820 | D | G | A | 0.842 | 0.283 | 0.158 | Glu266Lys |
| CARD4.2 | rs5743335 | D | A | T | 0.967 | 0.067 | 0.033 | Exon 3 |
| CARD4.3 | rs2906766 | D | A | G | 0.850 | 0.200 | 0.150 | Exon 2 |
| CARD15.1 | rs2066842 | D | C | T | 0.700 | 0.500 | 0.300 | Pro268Ser |
| CARD15.2 | rs5743277 | D | C | T | 0.992 | 0.017 | 0.008 | Arg703Cys |
| CARD15.3 | rs5743291 | D | G | A | 0.950 | 0.100 | 0.050 | Val955Ile |
| CARD15.4 | rs3135499 | D | A | C | 0.600 | 0.625 | 0.400 | 3′ UTR |

*Designations in the SNP-column are in-house SNP-names consisting of gene name and a sequential number. The RS-numbers are SNP-identification codes as applied in most public nucleic acid polymorphism databases. The Assay column indicates to which in-house multiplex nucleic acid polymorphism assay(s) the corresponding polymorphism belongs. The in-house multiplex nucleic acid polymorphism assays are named A, B, C and D. Some of the nucleic acid polymorphisms can be tested using one of two (or both) possible assays. Major allele is the most frequently observed allele at a locus, i.e. the wildtype-allele, while minor allele is a nucleic acid variant that occurs less frequently. Mutation indicates the location and/or effect of the polymorphism on the resulting receptor. The frequencies given are estimated population frequencies based on information disclosed in NCBI's databases.

TABLE 4
PCR-primer sequences* (SEQ ID Nos in brackets)

| | | | |
|---|---|---|---|
| | | | Size |
| Amplikon | Forward Primer (SEQ ID NO) | Reverse Primer (SEQ ID NO) | (bp) |

Assay A: TLR2, 4, 5, 9 assay (7 amplikons comprising 13 polymorphic loci)

| | | | |
|---|---|---|---|
| TLR2.1 | GAAAAATGAATGAGCAAGCAAA (1) | ATGGCCTCCTGCTTATGTCA (2) | 270 |
| TLR2.2 + 3 | GTTTCCATGGCCTGTGGTAT (3) | CAAAATCCTTCCCGCTGAG (4) | 493 |
| TLR4.1 | GGAGGAAGGGAGAAATGAGG (5) | CACCTCCAAAAGCTTCCTTG (6) | 208 |
| TLR4.2 + 3 | TGCAATTTGACCATTGAAGAA (7) | TCAAATTGGAATGCTGGAAA (8) | 463 |
| TLR5.1 | TCCCAAATGAAGGATGAAGG (9) | GCTCCTGCTGAGCTTCAACT (10) | 353 |
| TLR5.2 + 3 + 4 + 5 | CGGACTTGACAACCTCCAAG (11) | AAAGCATTCTGCACCCATGT (12) | 1134 |
| TLR9.1 + 2 | CCTGCTTGCAGTTGACTGTG (13) | GTGCTGGGCACTGTACTGG (14) | 379 |

Assay B: TLR3, 7, 8 assay (7 amplikons comprising 13 polymorphic loci)

| | | | |
|---|---|---|---|
| TLR3.1 | TGGCTAAAATGTTTGGAGCAC (15) | CCTGTGAGTTCTTGCCCAAT (16) | 290 |
| TLR7.1 | CGCATTTTAAAGCAATGATCC (17) | TGGTTGAAGAGAGCAGAGCA (18) | 156 |
| TLR7.2 | AGGCAGCAAATGGGAATTTT (19) | GAGTGACATCACAGGGCAGA (20) | 192 |
| TLR7.3 | TGAAGTTCTTGATCTTGGCACT (21) | TTTTTGAATCTGCAACTCCTTG (22) | 240 |
| TLR7.4 | ACCAATTGCTTCCGTGTCAT (23) | CTTTGCAGTGCAGATAAAAACA (24) | 276 |
| TLR8.1 + 2 + 3 | ATTTTCCAGCCTCACGAATG (25) | TCTGGGTCAGAAACCCCATA (26) | 744 |
| TLR8.4 | GTGTCTCAGAGGCTGCAATG (27) | GATGAAGCAAGCTGCCTTGT (28) | 162 |

Assay C: TLR1-10 assay (32 amplikons comprising 34 polymorphic loci)

| | | | |
|---|---|---|---|
| TLR1.1 | AAACGGTCTCATCCACGTTC (29) | CCAAGTGCTTGAGGTTCACA (30) | 260 |
| TLR1.2 | CATTGTGTTCCCCACAAACA (31) | CGAACACATCGCTGACAACT (32) | 359 |
| TLR1.3 | GGGTCAGCTGGACTTCAGAG (33) | AAAATCCAAATGCAGGAACG (34) | 214 |
| TLR1.4 | AGGGCTGGCCTGATTCTTAT (35) | GAAGGCAAATCTGCATACCTTC (36) | 363 |
| TLR2.1B | GAAAAATGAATGAGCAAGCAA (37) | GTCTTGCCAGAGGTTCATCA (38) | 188 |
| TLR2.2 | GTTTCCATGGCCTGTGGTAT (39) | TTCTCCACCCAGTAGGCATC (40) | 139 |
| TLR2.3 | CTGGAGCCCATTGAGAAAAA (41) | CAAAATCCTTCCCGCTGAG (42) | 116 |
| TLR3.1B | TTTGCGAACTTTGACAAATGA (43) | TCATTAAGGCCCAGGTCAAG (44) | 150 |
| TLR4.1 | GGAGGAAGGGAGAAATGAGG (45) | CACCTCCAAAAGCTTCCTTG (46) | 208 |
| TLR4.2 | TGCAATTTGACCATTGAAGAA (47) | TGGAAGTGAAAGTAAGCCTTTTG (48) | 241 |
| TLR4.3 | CAACAAAGGTGGGAATGCTT (49) | TCAAATTGGAATGCTGGAAA (50) | 224 |
| TLR5.1 | TCCCAAATGAAGGATGAAGG (51) | GCTCCTGCTGAGCTTCAACT (52) | 353 |
| TLR5.2 | CGGACTTGACAACCTCCAAG (53) | AAGTGGATGAGGTTCGCTGT (54) | 291 |
| TLR5.3 | GCTCCTAGCTCCTAATCCTGA (55) | AGAAGAGGGAAACCCCAGAG (56) | 191 |
| TLR5.4 | CACTATAGCTGGGCCTCCTG (57) | TCCTCTTCATCACAACCTTCC (58) | 102 |
| TLR5.5 | CGGGGCTTCTGTTTTATCTG (59) | AAAGCATTCTGCACCCATGT (60) | 143 |
| TLR6.1 | ACCCAGAACGTTTTCACAGA (61) | CCACATCCAGGAAGGTCAGT (62) | 395 |
| TLR6.2 | CGACATTGAAAGCATTGACA (63) | CCTTCGTCATGAGACCTACTTTG (64) | 297 |
| TLR6.3 | GAATGCAAAAACCCTTCACC (65) | CAGGCATTTCCAAGTCGTTT (66) | 217 |
| TLR7.1 | CGCATTTTAAAGCAATGATCC (67) | TGGTTGAAGAGAGCAGAGCA (68) | 156 |
| TLR7.2 | AGGCAGCAAATGGGAATTTT (69) | GAGTGACATCACAGGGCAGA (70) | 192 |
| TLR7.3 | TGAAGTTCTTGATCTTGGCACT (71) | TTTTTGAATCTGCAACTCCTTG (72) | 240 |
| TLR7.4 | ACCAATTGCTTCCGTGTCAT (73) | CTTTGCAGTGCAGATAAAAACA (74) | 276 |
| TLR8.1 | ATTTTCCAGCCTCACGAATG (75) | ACCGGTAGGTATGGGTCTCC (76) | 167 |
| TLR8.2 + 3 | CACAAGTTCCCTTCTTTTCATGT | TCTGGGTCAGAAACCCCATA (78) | 239 |
| | (77) | | |
| TLR8.4 | GTGTCTCAGAGGCTGCAATG (79) | GATGAAGCAAGCTGCCTTGT (80) | 162 |
| TLR9.1 | GCCATCCAGCCTTCTTACAA (81) | GTGCTGGGCACTGTACTGG (82) | 228 |
| TLR9.2 | CCTGCTTGCAGTTGACTGTG (83) | CCCTGTTGAGAGGGTGACAT (84) | 156 |
| TLR10.1 + 2 | GCCCAAGGATAGGCGTAAAT (85) | CCAACTTCCCAAGGACTGTG (86) | 191 |
| TLR10.3 | TGCTTGCCCAAAAGTATTCA (87) | GGCAGCTCTGAACAAAATCC (88) | 217 |
| TLR10.4 | AATGCACAAATGCCACACAT (89) | TCCAAGTGTTCCAAGGGTGT (90) | 208 |
| TLR10.5 | TTTGCGTGATGGAATCAAGA (91) | GGAAAAGGTCGTCCCAGAGT (92) | 164 |

Assay D: PRR assay (11 amplikons comprising 11 polymorphic loci)

| | | | |
|---|---|---|---|
| MDA5.1 | GGAAGGAATGCCGTGTAGAA (93) | ATCTGGCCCACAGCAATTTA (94) | 238 |
| MDA5.2 | GAGCCAGAGCTGATGAGAGC (95) | TATCAATGGCAACCACATGC (96) | 217 |
| MDA5.3 | CCCAAGGCAGCTCAATTACT (97) | GGAAGGGGAATCACTGGTTT (98) | 228 |
| DDX58.1 | CTCGGAAAATCCCTGCTTTC (99) | GGCCATGTAGCTCAGGATGT (100) | 199 |
| DDX58.2 | TGCACGAATGAAAGATGCTC (101) | TTGTTTTGCCATTCCAGTCA (102) | 168 |
| CARD4.1 | TAGACGCAGGGGTCAAATTC (103) | CTCAGGTCCAAGTCCGAGTG (104) | 217 |
| CARD4.2 | CATTCACCATGTGCCACAATA | CCATGGCAGTCAATTCTATGA (106) | 250 |
| | (105) | | |
| CARD4.3 | CTTGCCAGATGGCTGTCATA (107) | GCTTCCTTCATCCCTCCTTT (108) | 244 |
| CARD15.1 | GCTGCCACATGCAAGAAGTA (109) | CAGCACAGTGTCCGCATC (110) | 243 |
| CARD15.2 | AGATCACAGCAGCCTTCCTG (111) | GGATGGAGTGGAAGTGCTTG (112) | 153 |
| CARD15.3 | GGTCTTTCCCTGCTCTGACA (113) | GTCCACACAACCGCTCCTAT (114) | 218 |
| CARD15.4 | GCCATTGACTTCTTCCCAAG (115) | GGGAAAGCTGCTTCCTGAAT (116) | 239 |

*The Primer sequences are in the 5′ -> 3′ direction. The amplikon column indicates the locus amplified using the nomenclature of table 3 (i.e. amplikon TLR1.1 discloses primers for amplification of the polymorphic locus comprising rs5743611. Size is the predicted size in basepairs (bp) of the PCR-product that is produced, using the primers indicated.

TABLE 5
ASPE-primer sequences*

| | | | | |
|---|---|---|---|---|
| | | | | SEQ |
| | | FlexMAP- | | ID |
| Polymorphism | Allele | beadset | ASPE-primer | NO: |

Assay A: TLR2, 4, 5, 9 assay (13-plex)

| | | | | |
|---|---|---|---|---|
| TLR2.1 | Major | LUA-28 | CTACAAACAAACAAACATTATCAATAGTAAAATAAATCCAGA | 117 |
| RS1898830 | | | GAAATCA | |
| | Minor | LUA-70 | ATACCAATAATCCAATTCATATCATAGTAAAATAAATCCAGA | 118 |
| | | | GAAATCG | |
| TLR2.2 | Major | LUA-18 | TCAAAATCTCAAATACTCAAATCACAGGCCAAAAGGAAGCA | 119 |
| RS5743704 | Minor | LUA-41 | TTACTACACAATATACTCATCAATAGGCCAAAAGGAAGCC | 120 |
| TLR2.3 | Major | LUA-30 | TTACCTTTATACCTTTCTTTTTACGTCTTGGTGTTCATTATCT | 121 |
| RS5743708 | | | TCT | |
| | Minor | LUA-88 | TTACTTCACTTTCTATTTACAATCGTCTTGGTGTTCATTATCT | 122 |
| | | | TCC | |
| TLR4.1 | Major | LUA-72 | TCATTTACCTTTAATCCAATAATCCAGCTGTATAGCAGAGTT | 123 |
| RS7873784 | | | CG | |
| | Minor | LUA-7 | CAATTCATTTACCAATTTACCAATCAGCTGTATAGCAGAGTT | 124 |
| | | | CC | |
| TLR4.2 | Major | LUA-24 | TCAATTACCTTTTCAATACAATACATACTTAGACTACTACCT | 125 |
| RS4986790 | | | CGATGA | |
| | Minor | LUA-25 | CTTTTCAATTACTTCAAATCTTCACTTAGACTACTACCTCGA | 126 |
| | | | TGG | |
| TLR4.3 | Major | LUA-16 | AATCAATCTTCATTCAAATCATCATCAAAGTGATTTTGGGAC | 127 |
| RS4986791 | | | AAC | |
| | Minor | LUA-57 | CAATATCATCATCTTTATCATTACCTCAAAGTGATTTTGGGA | 128 |
| | | | CAAT | |
| TLR5.1 | Major | LUA-26 | TTACTCAAAATCTACACTTTTTCACTTGTCAATAGTCAAGGG | 129 |
| RS764535 | | | GA | |
| | Minor | LUA-23 | TTCAATCATTCAAATCTCAACTTTTTGTCAATAGTCAAGGGGG | 130 |
| TLR5.2 | Major | LUA-20 | CTTTTACAATACTTCAATACAATCAATTACAGACCTTGGATC | 131 |
| RS5744168 | | | TCC | |
| | Minor | LUA-67 | TCATTTACTCAACAATTACAAATCAAAAATTACAGACCTTGG | 132 |
| | | | ATCTCT | |
| TLR5.3 | Major | LUA-21 | AATCCTTTCTTTAATCTCAAATCAAATGTGAACTTAGCACTT | 133 |
| RS2072493 | | | TTATCAA | |
| | Minor | LUA-22 | AATCCTTTTTACTCAATTCAATCAAATGTGAACTTAGCACTT | 134 |
| | | | TTATCAG | |
| TLR5.4 | Major | LUA-49 | TCATCAATCTTTCAATTTACTTACGTGTACCCTGACTCGC | 135 |
| RS5744174 | Minor | LUA-33 | TCAATTACTTCACTTTAATCCTTTTGTGTACCCTGACTCGT | 136 |
| TLR5.5 | Major | LUA-80 | CTAACTAACAATAATCTAACTAACCAGAACCTGATATGTACA | 137 |
| RS5744176 | | | AATATGA | |
| | Minor | LUA-96 | ATACTAACTCAACTAACTTTAAACCAGAACCTGATATGTACA | 138 |
| | | | AATATGG | |
| TLR9.1 | Major | LUA-64 | CTACATATTCAAATTACTACTTACAGATAAAAGATCACTGCC | 139 |
| RS187084 | | | CTT | |
| | Minor | LUA-82 | TACATACACTAATAACATACTCATAGATAAAAGATCACTGCC | 140 |
| | | | CTC | |
| TLR9.2 | Major | LUA-2 | CTTTATCAATACATACTACAATCAGAGACTTGGGGGAGTTTT | 141 |
| RS5743836 | | | | |
| | Minor | LUA-38 | TCAATCATTACACTTTTCAACAATAGACTTGGGGGAGTTTC | 142 |

Assay B: TLR3, 7, 8 assay (9-plex)

| | | | | |
|---|---|---|---|---|
| TLR3.1 | Major | LUA-12 | TACACTTTCTTTCTTTCTTTCTTTAGATTTTATTCTTGGTTAG | 143 |
| RS3775291 | | | GTTGAG | |
| | Minor | LUA-56 | CAATTTACTCATATACATCACTTTAGATTTTATTCTTGGTTAG | 144 |
| | | | GTTGAA | |
| TLR7.1 | Major | LUA-65 | CTTTTCATCAATAATCTTACCTTTGTGCTGTCTTTGAAATGT | 145 |
| RS2302267 | | | AAACTTT | |
| | Minor | LUA-47 | CTTCTCATTAACTTACTTCATAATTGCTGTCTTTGAAATGTA | 146 |
| | | | AACTTG | |
| TLR7.2 | Major | LUA-76 | AATCTAACAAACTCATCTAAATACGTGGACACTGAAGAGACA | 147 |
| RS179008 | Minor | LUA-77 | CAATTAACTACATACAATACATACGTGGACACTGAAGAGACT | 148 |
| TLR7.3 | Major | LUA-98 | AATCATACTCAACTAATCATTCAACATAACTTTCTACAGAAG | 149 |
| RS5743781 | | | TTCTGG | |
| | Minor | LUA-50 | CAATATACCAATATCATCATTTACTCATAACTTTCTACAGAA | 150 |
| | | | GTTCTGA | |
| TLR7.4 | Major | LUA-34 | TCATTCATATACATACCAATTCATAAGCAGGCCCAAGG | 151 |
| RS3853839 | Minor | LUA-9 | TAATCTTCTATATCAACATCTTACAAGCAGGCCCAAGC | 152 |
| TLR8.1 | Major | LUA-90 | CTAAATACTTCACAATTCATCTAAAACACTCATTGAGCTTAT | 153 |
| RS5741883 | | | ACTACAC | |
| | Minor | LUA-40 | CTTTCTACATTATTCACAACATTAAACACTCATTGAGCTTAT | 154 |
| | | | ACTACAT | |
| TLR8.2 | Major | LUA-48 | AAACAAACTTCACATCTCAATAATACTTCTGTAAAACACACG | 155 |
| RS3764879 | | | CTAC | |
| | Minor | LUA-62 | TCAATCATAATCTCATAATCCAATACTTCTGTAAAACACACG | 156 |
| | | | CTAG | |
| TLR8.3 | Major | LUA-87 | AAACTAACATCAATACTTACATCAATGAAAAATTAGAACAAC | 157 |
| RS3764880 | | | AGAAACA | |
| | Minor | LUA-89 | TATACTATCAACTCAACAACATATATGAAAAATTAGAACAAC | 158 |
| | | | AGAAACG | |
| TLR8.4 | Major | LUA-32 | ATTATTCACTTCAAACTAATCTACGGATTCAATTCCTCCTGG | 159 |
| RS5744088 | Minor | LUA-55 | TATATACACTTCTCAATAACTAACGGATTCAATTCCTCCTGC | 160 |

Assay C: TLR1-10 assay (34-plex)

| | | | | |
|---|---|---|---|---|
| TLR1.1 | Major | LUA-95 | TACACTTTAAACTTACTACACTAAACACTGATATCAAGATAC | 161 |
| RS5743611 | | | TGGATTG | |
| | Minor | LUA-63 | CTACTTCATATACTTTATACTACAACACTGATATCAAGATAC | 162 |
| | | | TGGATTC | |
| TLR1.2 | Major | LUA-5 | CAATTCAAATCACAATAATCAATCTTCAAACAAATCCAAAGT | 163 |
| RS4833095 | | | TATCAAA | |
| | Minor | LUA-60 | AATCTACAAATCCAATAATCTCATTTCAAACAAATCCAAAGT | 164 |
| | | | TATCAAG | |
| TLR1.3 | Major | LUA-94 | CTTTCTATCTTTCTACTCAATAATCTTGAAGGCCTTGTCTAT | 165 |
| RS3923647 | | | ACA | |
| | Minor | LUA-84 | TCAACTAACTAATCATCTATCAATCTTGAAGGCCTTGTCTAT | 166 |
| | | | ACT | |
| TLR1.4 | Major | LUA-6 | TCAACAATCTTTTACAATCAAATCGGGCAGATCCAAGTAGC | 167 |
| RS5743618 | Minor | LUA-51 | TCATTTCAATCAATCATCAACAATAGGGCAGATCCAAGTAGA | 168 |
| TLR2.1 | Major | LUA-28 | CTACAAACAAACAAACATTATCAATAGTAAAATAAATCCAGA | 169 |
| RS1898830 | | | GAAATCA | |
| | Minor | LUA-70 | ATACCAATAATCCAATTCATATCATAGTAAAATAAATCCAGA | 170 |
| | | | GAAATCG | |
| TLR2.2 | Major | LUA-18 | TCAAAATCTCAAATACTCAAATCACAGGCCAAAAGGAAGCA | 171 |
| RS5743704 | Minor | LUA-41 | TTACTACACAATATACTCATCAATAGGCCAAAAGGAAGCC | 172 |
| TLR2.3 | Major | LUA-30 | TTACCTTTATACCTTTCTTTTTACGTCTTGGTGTTCATTATCT | 173 |
| RS5743708 | | | TCT | |
| | Minor | LUA-88 | TTACTTCACTTTCTATTTACAATCGTCTTGGTGTTCATTATCT | 174 |
| | | | TCC | |
| TLR3.1 | Major | LUA-12 | TACACTTTCTTTCTTTCTTTCTTTAGATTTTATTCTTGGTTAG | 175 |
| RS3775291 | | | GTTGAG | |
| | Minor | LUA-56 | CAATTTACTCATATACATCACTTTAGATTTTATTCTTGGTTAG | 176 |
| | | | GTTGAA | |
| TLR4.1 | Major | LUA-7 | CAATTCATTTACCAATTTACCAATCAGCTGTATAGCAGAGTT | 177 |
| RS7873784 | | | CC | |
| | Minor | LUA-72 | TCATTTACCTTTAATCCAATAATCCAGCTGTATAGCAGAGTT | 178 |
| | | | CG | |
| TLR4.2 | Major | LUA-24 | TCAATTACCTTTTCAATACAATACATACTTAGACTACTACCT | 179 |
| RS4986790 | | | CGATGA | |
| | Minor | LUA-25 | CTTTTCAATTACTTCAAATCTTCACTTAGACTACTACCTCGA | 180 |
| | | | TGG | |
| TLR4.3 | Major | LUA-16 | AATCAATCTTCATTCAAATCATCATCAAAGTGATTTTGGGAC | 181 |
| RS4986791 | | | AAC | |
| | Minor | LUA-57 | CAATATCATCATCTTTATCATTACCTCAAAGTGATTTTGGGA | 182 |
| | | | CAAT | |
| TLR5.1 | Major | LUA-26 | TTACTCAAAATCTACACTTTTTCACTTGTCAATAGTCAAGGG | 183 |
| RS764535 | | | GA | |
| | Minor | LUA-23 | TTCAATCATTCAAATCTCAACTTTTTGTCAATAGTCAAGGGGG | 184 |
| TLR5.2 | Major | LUA-20 | CTTTTACAATACTTCAATACAATCAATTACAGACCTTGGATC | 185 |
| RS5744168 | | | TCC | |
| | Minor | LUA-67 | TCATTTACTCAACAATTACAAATCAAAAATTACAGACCTTGG | 186 |
| | | | ATCTCT | |
| TLR5.3 | Major | LUA-21 | AATCCTTTCTTTAATCTCAAATCAAATGTGAACTTAGCACTT | 187 |
| RS2072493 | | | TTATCAA | |
| | Minor | LUA-22 | AATCCTTTTTACTCAATTCAATCAAATGTGAACTTAGCACTT | 188 |
| | | | TTATCAG | |
| TLR5.4 | Major | LUA-49 | TCATCAATCTTTCAATTTACTTACGTGTACCCTGACTCGC | 189 |
| RS5744174 | Minor | LUA-33 | TCAATTACTTCACTTTAATCCTTTTGTGTACCCTGACTCGT | 190 |
| TLR5.5 | Major | LUA-80 | CTAACTAACAATAATCTAACTAACCAGAACCTGATATGTACA | 191 |
| RS5744176 | | | AATATGA | |
| | Minor | LUA-96 | ATACTAACTCAACTAACTTTAAACCAGAACCTGATATGTACA | 192 |
| | | | AATATGG | |
| TLR6.1 | Major | LUA-46 | TACATCAACAATTCATTCAATACAAAATTTACACCACTATAC | 193 |
| RS5743815 | | | TCTCAG | |
| | Minor | LUA-19 | TCAATCAATTACTTACTCAAATACAAATTTACACCACTATAC | 194 |
| | | | TCTCAA | |
| TLR6.2 | Major | LUA-54 | CTTTTTCAATCACTTTCAATTCATACCATTTCAGATACACCTT | 195 |
| RS5743813 | | | TTATAC | |
| | Minor | LUA-8 | AATCCTTTTACATTCATTACTTACACCATTTCAGATACACCTT | 196 |
| | | | TTATAT | |
| TLR6.3 | Major | LUA-69 | CTATAAACATATTACATTCACATCGGGTAAAATTCAGTAAGG | 197 |
| RS5743810 | | | TTGG | |
| | Minor | LUA-93 | CTTTCTATTCATCTAAATACAAACAGGGTAAAATTCAGTAAG | 198 |
| | | | GTTGA | |
| TLR7.1 | Major | LUA-47 | CTTCTCATTAACTTACTTCATAATTGCTGTCTTTGAAATGTA | 199 |
| RS2302267 | | | AACTTG | |
| | Minor | LUA-65 | CTTTTCATCAATAATCTTACCTTTGTGCTGTCTTTGAAATGT | 200 |
| | | | AAACTTT | |
| TLR7.2 | Major | LUA-76 | AATCTAACAAACTCATCTAAATACGTGGACACTGAAGAGACA | 201 |
| RS179008 | Minor | LUA-77 | CAATTAACTACATACAATACATACGTGGACACTGAAGAGACT | 202 |
| TLR7.3 | Major | LUA-98 | AATCATACTCAACTAATCATTCAACATAACTTTCTACAGAAG | 203 |
| RS5743781 | | | TTCTGG | |
| | Minor | LUA-50 | CAATATACCAATATCATCATTTACTCATAACTTTCTACAGAA | 204 |
| | | | GTTCTGA | |
| TLR7.4 | Major | LUA-34 | TCATTCATATACATACCAATTCATAAGCAGGCCCAAGG | 205 |
| RS3853839 | Minor | LUA-9 | TAATCTTCTATATCAACATCTTACAAGCAGGCCCAAGC | 206 |
| TLR8.1 | Major | LUA-90 | CTAAATACTTCACAATTCATCTAAAACACTCATTGAGCTTAT | 207 |
| RS5741883 | | | ACTACAC | |
| | Minor | LUA-40 | CTTTCTACATTATTCACAACATTAAACACTCATTGAGCTTAT | 208 |
| | | | ACTACAT | |
| TLR8.2 | Major | LUA-48 | AAACAAACTTCACATCTCAATAATACTTCTGTAAAACACACG | 209 |
| RS3764879 | | | CTAC | |
| | Minor | LUA-62 | TCAATCATAATCTCATAATCCAATACTTCTGTAAAACACACG | 210 |
| | | | CTAG | |
| TLR8.3 | Major | LUA-87 | AAACTAACATCAATACTTACATCAATGAAAAATTAGAACAAC | 211 |
| RS3764880 | | | AGAAACA | |
| | Minor | LUA-89 | TATACTATCAACTCAACAACATATATGAAAAATTAGAACAAC | 212 |
| | | | AGAAACG | |
| TLR8.4 | Major | LUA-55 | TATATACACTTCTCAATAACTAACGGATTCAATTCCTCCTGC | 213 |
| RS5744088 | Minor | LUA-32 | ATTATTCACTTCAAACTAATCTACGGATTCAATTCCTCCTGG | 214 |
| TLR9.1 | Major | LUA-64 | CTACATATTCAAATTACTACTTACAGATAAAAGATCACTGCC | 215 |
| RS187084 | | | CTT | |
| | Minor | LUA-82 | TACATACACTAATAACATACTCATAGATAAAAGATCACTGCC | 216 |
| | | | CTC | |
| TLR9.2 | Major | LUA-2 | CTTTATCAATACATACTACAATCAGAGACTTGGGGGAGTTTT | 217 |
| RS5743836 | Minor | LUA-38 | TCAATCATTACACTTTTCAACAATAGACTTGGGGGAGTTTC | 218 |
| TLR10.1 | Major | LUA-86 | CTAATTACTAACATCACTAACAATACAGAGTTAAATGAAGA | 219 |
| RS4129008 | | | GTCTCA | |
| | Minor | LUA-71 | ATCATTACAATCCAATCAATTCATACAGAGTTAAATGAAGAG | 220 |
| | | | TCTCG | |
| TLR10.2 | Major | LUA-29 | AATCTTACTACAAATCCTTTCTTTTGGTGGCTAATACATTAA | 221 |
| RS4129009 | | | CATTAAT | |
| | Minor | LUA-79 | TTCATAACTACAATACATCATCATTGGTGGCTAATACATTAA | 222 |
| | | | CATTAAC | |
| TLR10.3 | Major | LUA-39 | TACACAATCTTTTCATTACATCATAGATCAGTTAGAAATTAA | 223 |
| RS11466657 | | | ATGCAG | |
| | Minor | LUA-27 | CTTTTCAAATCAATACTCAACTTTGATGGCCTTACGAGAACT | 224 |
| | | | AAATAT | |
| TLR10.4 | Major | LUA-99 | AATCTACACTAACAATTTCATAACCAGACGAGTTGTTTAAAA | 225 |
| RS11096955 | | | GAACTA | |
| | Minor | LUA-83 | ATACAATCTAACTTCACTATTACAAGACGAGTTGTTTAAAAG | 226 |
| | | | AACTC | |
| TLR10.5 | Major | LUA-11 | TACAAATCATCAATCACTTTAATCATGCAACGAAATCTTAGT | 227 |
| RS11096957 | | | TTAGAAA | |
| | Minor | LUA-52 | TCAATCATCTTTATACTTCACAATATGCAACGAAATCTTAGT | 228 |
| | | | TTAGAAC | |

Assay D: PRR-assay (11-plex)

| | | | | |
|---|---|---|---|---|
| MDA5.3 | Major | LUA-85 | ATACTACATCATAATCAAACATCAGGGAACTTTACATTGTAA | 229 |
| RS1990760 | | | GAGAAAACAAAA | |
| | Minor | LUA-70 | ATACCAATAATCCAATTCATATCAGGGAACTTTACATTGTAA | 230 |
| | | | GAGAAAACAAAG | |
| MDA5.2 | Major | LUA-68 | TCATAATCTCAACAATCTTTCTTTTTCTCTCGGAAATCATTAA | 231 |
| RS3747517 | | | CTGTCTCAT | |
| | Minor | LUA-78 | CTATCTATCTAACTATCTATATCACTCTCGGAAATCATTAAC | 232 |
| | | | TGTCTCAC | |
| MDA5.1 | Major | LUA-29 | AATCTTACTACAAATCCTTTCTTTTATTGTTTTTCAACTTCTG | 233 |
| RS10930046 | | | CATCAAATAAT | |
| | Minor | LUA-59 | TCATCAATCAATCTTTTTCACTTTTATTGTTTTTCAACTTCTG | 234 |
| | | | CATCAAATAAC | |
| DDX58.1 | Major | LUA-76 | AATCTAACAAACTCATCTAAATACGACCACCGAGCAGCGAC | 235 |
| RS10813831 | Minor | LUA-77 | CAATTAACTACATACAATACATACATGACCACCGAGCAGCG | 236 |
| | | | AT | |
| DDX58.2 | Major | LUA-33 | TCAATTACTTCACTTTAATCCTTTCTCTGAGTAAGATCTTGC | 237 |
| RS17217280 | | | TCAATCTCT | |
| | Minor | LUA-31 | TTCACTTTTCAATCAACTTTAATCCTCTGAGTAAGATCTTGC | 238 |
| | | | TCAATCTCA | |
| CARD4.1 | Major | LUA-5 | CAATTCAAATCACAATAATCAATCAGGAAGGCAAACACCTC | 239 |
| RS2075820 | | | CTT | |
| | Minor | LUA-99 | AATCTACACTAACAATTTCATAACAGGAAGGCAAACACCTC | 240 |
| | | | CTC | |
| CARD4.2 | Major | LUA-82 | TACATACACTAATAACATACTCATAGATTCAGAAATAAATTG | 241 |
| RS5743335 | | | GAAATTGAAGA | |
| | Minor | LUA-28 | CTACAAACAAACAAACATTATCAAAGATTCAGAAATAAATTG | 242 |
| | | | GAAATTGAAGT | |
| CARD4.3 | Major | LUA-87 | AAACTAACATCAATACTTACATCATGGCGGCGATTACAGAA | 243 |
| RS2906766 | | | AACA | |
| | Minor | LUA-10 | ATCATACATACATACAAATCTACAGGCGGCGATTACAGAAA | 244 |
| | | | ACG | |
| CARD15.1 | Major | LUA-1 | CTTTAATCTCAATCAATACAAATCATGTGGGCATGGCTGGAC | 245 |
| RS2066842 | Minor | LUA-30 | TTACCTTTATACCTTTCTTTTTACGATGTGGGCATGGCTGGAT | 246 |
| CARD15.2 | Major | LUA-84 | TCAACTAACTAATCATCTATCAATGGCACAGGCCTGGCG | 247 |
| RS5743277 | Minor | LUA-96 | ATACTAACTCAACTAACTTTAAACGGCACAGGCCTGGCA | 248 |
| CARD15.3 | Major | LUA-44 | TCATTTACCAATCTTTCTTTATACCTCACCAGAGTTCTTCTA | 249 |
| RS5743291 | | | GCATGAT | |
| | Minor | LUA-7 | CAATTCATTTACCAATTTACCAATTCACCAGAGTTCTTCTAG | 250 |
| | | | CATGAC | |
| CARD15.4 | Major | LUA-18 | TCAAAATCTCAAATACTCAAATCACCTCTCACAAAAGACCCC | 251 |
| RS3135499 | | | TTA | |
| | Minor | LUA-60 | AATCTACAAATCCAATAATCTCATCCTCTCACAAAAGACCCC | 252 |
| | | | TTC | |

*The Primer sequences are in the 5′ -> 3′ direction. The Allele column indicates whether the corresponding ASPE-primer is specific for the most frequently observed allele (Major) or the least frequently observed allele (Minor). The FlexMAP-beadset column indicates which particular FlexMAP-beadset the corresponding ASPE-primers 5′-end contains the complementary tag-sequence for. The ASPE-primer column discloses the oligonucleotide sequence of the ASPE-primer where the first twenty-four 5′-end nucleotides constitutes the tag-sequence and the rest of the sequence constitutes the allele-specific part of the primer.

TABLE 6
Genotypes for control samples*

| | | | | | | | | | RH- | RH- | RH- | RH- |
|---|---|---|---|---|---|---|---|---|---|---|---|---|
| | | | | | | | | | person | person | person | person |
| SNP | rs# | NA06990 | NA07019 | NA10831 | NA10843 | NA10844 | NA10861 | NA12560 | 2 | 4 | 8 | 13 |
| TLR1.1 | rs5743611 | G/G | G/G | G/C | G/G | G/C | G/G | G/G | G/G | G/G | G/C | G/C |
| TLR1.2 | rs4833095 | G/G | A/A | A/A | A/A | A/A | A/A | A/G | A/G | A/G | A/G | A/A |
| TLR1.3 | rs3923647 | A/T | A/A | A/A | A/A | A/A | A/A | A/A | A/A | A/A | A/A | A/A |
| TLR1.4 | rs5743618 | T/T | G/G | G/G | G/G | G/G | G/G | G/T | G/T | G/T | G/T | G/G |
| TLR2.1 | rs1898830 | G/G | A/A | A/G | A/A | G/G | A/G | G/G | G/G | A/G | A/G | A/A |
| TLR2.2 | rs5743704 | C/C | C/C | C/C | C/A | C/C | C/C | C/C | C/C | C/C | C/C | C/C |
| TLR2.3 | rs5743708 | G/G | G/G | G/G | G/G | G/A | G/G | G/G | G/G | G/G | G/G | G/G |
| TLR3.1 | rs3775291 | G/G | G/G | G/A | A/A | G/A | G/G | G/A | A/A | G/G | A/A | G/G |
| TLR4.1 | rs7873784 | G/C | G/C | G/G | G/G | G/C | G/G | G/C | G/C | G/G | G/G | G/G |
| TLR4.2 | rs4986790 | A/A | A/A | A/A | A/A | A/A | A/A | A/A | A/A | A/G | A/G | A/A |
| TLR4.3 | rs4986791 | C/C | C/C | C/C | C/C | C/C | C/C | C/C | C/C | C/T | C/T | C/C |
| TLR5.1 | rs764535 | G/G | G/G | G/G | G/G | G/G | G/G | G/G | G/G | G/G | G/G | G/G |
| TLR5.2 | rs5744168 | C/C | C/T | C/C | C/C | C/C | C/C | C/C | C/C | C/C | C/C | C/C |
| TLR5.3 | rs2072493 | A/A | A/A | A/G | A/G | A/G | A/A | A/A | A/A | A/A | A/A | A/G |
| TLR5.4 | rs5744174 | T/T | C/C | T/T | T/T | T/T | C/C | T/T | C/C | T/C | T/T | T/T |
| TLR5.5 | rs5744176 | A/A | A/A | A/A | A/A | A/A | A/G | A/A | A/A | A/A | A/A | A/A |
| TLR6.1 | rs5743815 | T/T | T/T | T/T | T/C | T/C | T/T | T/C | T/C | T/T | T/T | T/T |
| TLR6.2 | rs5743813 | C/C | C/T | C/C | C/C | C/C | C/C | C/C | C/C | C/C | C/C | C/C |
| TLR6.3 | rs5743810 | C/C | C/T | C/T | C/T | C/C | C/T | C/C | C/C | C/T | C/C | C/T |
| TLR7.1 | rs2302267 | T/T | T/T | T/T | T/G | T/T | T/T | T/T | T/T | T/T | T/T | T/T |
| TLR7.2 | rs179008 | A/A | A/A | T/A | A/A | A/A | A/A | A/A | A/A | T/A | T/T | A/A |
| TLR7.3 | rs5743781 | C/C | C/C | C/C | C/C | C/C | C/C | T/T | C/C | C/C | C/C | C/C |
| TLR7.4 | rs3853839 | C/G | G/G | G/G | G/G | G/G | G/G | G/G | C/C | G/G | G/G | C/C |
| TLR8.1 | rs5741883 | C/T | C/T | T/T | C/T | C/C | C/T | C/C | C/C | C/C | T/T | C/T |
| TLR8.2 | rs3764879 | C/G | C/C | C/C | C/C | C/C | C/C | C/C | G/G | C/C | C/C | C/G |
| TLR8.3 | rs3764880 | A/G | A/A | A/A | A/A | A/A | A/A | A/A | G/G | A/A | A/A | A/G |
| TLR8.4 | rs5744088 | G/G | G/G | G/C | G/C | G/G | G/G | G/G | G/G | G/G | C/C | G/C |
| TLR9.1 | rs187084 | T/C | T/C | T/T | T/C | T/C | T/T | T/C | T/T | C/C | T/T | C/C |
| TLR9.2 | rs5743836 | T/C | T/T | T/T | T/T | T/T | T/C | T/T | T/T | T/T | T/T | T/T |
| TLR10.1 | rs4129008 | G/G | G/G | G/G | G/G | G/G | G/G | G/G | G/G | G/G | G/G | G/G |
| TLR10.2 | rs4129009 | A/G | A/A | A/A | A/A | A/A | A/A | A/G | A/G | A/G | A/G | A/A |
| TLR10.3 | rs11466657 | T/T | T/T | T/T | T/T | T/T | T/T | T/C | T/C | T/T | T/T | T/T |
| TLR10.4 | rs11096955 | C/C | A/A | A/C | A/A | A/C | A/A | A/C | A/C | A/C | C/C | A/C |
| TLR10.5 | rs11096957 | C/C | A/A | A/C | A/A | A/C | A/A | A/C | A/C | A/C | C/C | A/C |
| MDA5.1 | rs10930046 | A/A | A/A | A/A | A/A | A/A | A/A | A/A | A/A | A/A | A/A | A/A |
| MDA5.2 | rs3747517 | C/T | C/T | C/C | C/C | C/T | C/C | C/T | C/C | T/T | C/C | C/C |
| MDA5.3 | rs1990760 | T/C | T/C | T/C | T/T | T/C | T/T | T/C | T/T | C/C | T/T | T/T |
| DDX58.1 | rs10813831 | G/G | G/A | G/G | G/A | G/G | G/A | G/G | G/G | G/G | G/A | A/A |
| DDX58.2 | rs17217280 | T/A | T/T | T/T | T/T | T/T | T/T | T/T | T/T | T/A | T/T | T/T |
| CARD4.1 | rs2075820 | G/G | G/G | G/G | G/G | G/G | G/G | G/G | G/G | G/A | G/G | G/A |
| CARD4.2 | rs5743335 | A/T | A/T | A/A | A/A | A/A | A/A | A/A | A/A | A/A | A/A | A/A |
| CARD4.3 | rs2906766 | A/G | A/G | A/A | A/A | A/A | A/A | A/A | A/A | G/G | A/A | A/G |
| CARD15.1 | rs2066842 | C/T | T/T | C/C | C/C | C/T | C/T | C/C | C/T | C/C | C/C | T/T |
| CARD15.2 | rs5743277 | C/C | C/C | C/C | C/C | C/C | C/C | C/C | C/C | C/C | C/C | C/C |
| CARD15.3 | rs5743291 | G/G | G/G | G/G | G/G | G/G | G/G | G/G | G/G | G/G | G/A | G/G |
| CARD15.4 | rs3135499 | A/C | A/A | A/A | A/A | A/A | A/C | A/C | A/C | A/C | C/C | A/C |

*The NA-codes are sample ID-codes from the Coriell Cell Repository (CCR), these samples have been sequenced by other groups. Genotypes for the CCR-samples were retrieved from the dbSNP database at NCBI. RH-persons are DNA-samples derived from employees at the National University Hospital of Denmark. Genotypes written in bold were obtained by sequencing at MWG-Biotech AG. Genotypes written in italics were only genotyped using the multiplexed bead-based assays. All other genotypes were retrieved from the NCBI databases.

TABLE 7A, B, C
Table 7a. Association analyses results*

| | Case-control | | | | | | Age at disease | |
|---|---|---|---|---|---|---|---|---|
| | analysis | | Clinical course | | MSSS | | onset | |
| SNP | Men | Women | Men | Women | Men | Women | Men | Women |
| TLR1.1 | − | − | − | − | − | − | − | − |
| TLR1.3 | − | − | − | − | − | − | − | − |
| TLR1.4 | − | − | − | − | − | + | − | − |
| TLR2.1 | − | − | − | − | − | − | − | − |
| TLR2.2 | − | − | − | − | − | − | − | − |
| TLR2.3 | − | − | − | − | − | − | − | − |
| TLR3.1 | − | + | − | − | − | − | − | + |
| TLR4.1 | − | − | − | ++ | − | − | − | − |
| TLR4.2 | − | − | + | − | − | − | − | − |
| TLR5.1 | − | − | − | − | − | − | − | − |
| TLR5.2 | − | − | − | − | − | − | − | − |
| TLR5.3 | − | − | − | − | − | − | − | − |
| TLR5.4 | − | − | − | − | + | − | − | − |
| TLR5.5 | N/A | N/A | N/A | N/A | N/A | N/A | N/A | N/A |
| TLR6.2 | N/A | N/A | N/A | N/A | N/A | N/A | N/A | N/A |
| TLR6.3 | − | − | − | − | + | − | − | − |
| TLR7.1 | − | − | − | − | − | − | − | − |
| TLR7.2 | − | − | − | − | − | − | + | − |
| TLR7.3 | N/A | − | N/A | N/A | N/A | N/A | N/A | N/A |
| TLR7.4 | − | − | − | − | − | − | − | − |
| TLR8.1 | − | − | − | − | − | − | − | − |
| TLR8.2 | − | − | − | − | − | − | − | − |
| TLR8.4 | − | − | − | − | − | − | − | − |
| TLR9.1 | − | − | − | − | − | − | − | − |
| TLR9.2 | − | − | − | − | − | − | − | − |
| TLR10.1 | − | − | − | − | − | − | − | − |
| TLR10.2 | − | − | − | − | − | − | − | − |
| TLR10.3 | − | − | − | − | − | − | − | − |
| TLR10.4 | − | − | − | − | − | − | − | − |
| TLR10.5 | − | − | − | − | − | − | − | − |
| MDA5.1 | − | − | − | − | − | − | − | − |
| MDA5.2 | − | + | − | − | + | − | − | − |
| MDA5.3 | − | − | − | − | + | − | − | − |
| DDX58.1 | − | − | − | − | − | − | − | − |
| DDX58.2 | − | − | − | − | − | − | − | + |
| CARD4.0 | N/A | N/A | N/A | N/A | N/A | N/A | N/A | N/A |
| CARD4.1 | − | − | − | − | − | + | − | + |
| CARD4.2 | − | − | − | − | − | − | − | − |
| CARD4.3 | − | − | − | − | − | − | − | − |
| CARD15.1 | − | − | − | − | − | − | − | − |
| CARD15.2 | − | − | − | − | − | − | − | − |
| CARD15.3 | + | − | − | + | − | − | − | − |
| CARD15.4 | − | − | − | − | − | − | − | − |

Table 7b. Association analyses results*

| | | | | | | | Neutralizing | |
|---|---|---|---|---|---|---|---|---|
| | | | | | | | antibodies | |
| | | | | | Neutralizing | | after 24 | |
| | Attacks 24 | | Steroid attacks | | antibodies after | | months of | |
| | months before | | 24 months | | 12 months of | | IFN- | |
| | IFN | | before IFN | | IFN-treatment | | treatment | |
| SNP | Men | Women | Men | Women | Men | Women | Men | Women |
| TLR1.1 | − | − | − | + | − | − | − | − |
| TLR1.3 | − | ++ | − | − | − | − | − | − |
| TLR1.4 | − | − | + | − | − | − | − | − |
| TLR2.1 | − | − | − | − | − | − | − | − |
| TLR2.2 | − | − | − | − | − | − | + | − |
| TLR2.3 | − | − | − | − | − | − | − | − |
| TLR3.1 | − | − | − | − | − | − | − | − |
| TLR4.1 | − | − | − | − | − | − | − | − |
| TLR4.2 | − | − | − | − | − | − | − | − |
| TLR5.1 | − | − | − | − | − | − | − | − |
| TLR5.2 | − | − | − | − | − | − | − | − |
| TLR5.3 | − | − | − | − | − | − | − | − |
| TLR5.4 | − | − | − | − | − | − | − | − |
| TLR5.5 | N/A | N/A | N/A | N/A | N/A | N/A | N/A | N/A |
| TLR6.2 | N/A | N/A | N/A | N/A | N/A | N/A | N/A | N/A |
| TLR6.3 | − | − | − | − | ++ | − | ++ | − |
| TLR7.1 | − | − | − | − | − | − | − | − |
| TLR7.2 | − | − | − | − | − | − | − | − |
| TLR7.3 | N/A | N/A | N/A | N/A | N/A | N/A | N/A | N/A |
| TLR7.4 | − | − | − | − | − | − | − | − |
| TLR8.1 | − | + | − | − | − | − | + | + |
| TLR8.2 | − | − | − | − | − | − | − | − |
| TLR8.4 | − | − | − | − | − | − | − | − |
| TLR9.1 | − | − | − | − | − | − | + | − |
| TLR9.2 | − | − | − | − | − | − | − | − |
| TLR10.1 | − | + | − | − | N/A | − | N/A | − |
| TLR10.2 | − | − | − | − | − | − | − | − |
| TLR10.3 | − | − | − | − | − | − | − | − |
| TLR10.4 | − | − | − | − | ++ | − | + | − |
| TLR10.5 | − | − | − | − | ++ | − | + | − |
| MDA5.1 | − | − | − | − | − | − | − | − |
| MDA5.2 | − | − | − | − | − | − | − | − |
| MDA5.3 | − | − | − | − | − | − | − | − |
| DDX58.1 | − | − | − | − | − | − | − | − |
| DDX58.2 | ++ | − | − | − | − | − | − | + |
| CARD4.0 | N/A | N/A | N/A | N/A | N/A | N/A | N/A | N/A |
| CARD4.1 | − | − | − | ++ | − | − | − | − |
| CARD4.2 | − | − | − | ++ | − | − | − | − |
| CARD4.3 | − | − | − | ++ | − | − | − | − |
| CARD15.1 | − | − | − | − | − | − | − | − |
| CARD15.2 | − | − | − | − | − | − | N/A | N/A |
| CARD15.3 | − | − | − | − | − | − | − | − |
| CARD15.4 | − | − | − | − | − | − | − | + |

Table 7c. Association analyses results*

| | | | Steroid-attacks | | | | | |
|---|---|---|---|---|---|---|---|---|
| | Attacks after | | after IFN- | | | | | |
| | IFN-treatment | | treatment | | | | | |
| | initiation | | initiation | | Time to first | | | |
| | normalized to | | normalized to | | attack after IFN- | | IFN- | |
| | observation- | | observation- | | treatment | | respondership | |
| | period | | period | | initiation | | (Yes/No) | |
| SNP | Men | Women | Men | Women | Men | Women | Men | Women |
| TLR1.1 | − | − | − | − | − | − | − | − |
| TLR1.3 | − | − | − | − | − | + | − | − |
| TLR1.4 | − | − | − | − | − | − | − | − |
| TLR2.1 | − | − | − | − | − | − | − | − |
| TLR2.2 | − | − | − | − | − | − | − | − |
| TLR2.3 | − | − | − | − | − | − | − | − |
| TLR3.1 | − | − | − | − | − | − | − | − |
| TLR4.1 | − | − | − | − | − | − | − | − |
| TLR4.2 | + | − | − | − | − | − | − | − |
| TLR5.1 | − | − | − | − | − | − | − | − |
| TLR5.2 | − | − | − | − | − | − | − | − |
| TLR5.3 | − | − | − | − | − | − | − | − |
| TLR5.4 | − | − | − | − | − | − | − | − |
| TLR5.5 | N/A | N/A | N/A | N/A | N/A | N/A | N/A | N/A |
| TLR6.2 | N/A | N/A | N/A | N/A | N/A | N/A | N/A | N/A |
| TLR6.3 | − | − | − | − | − | − | − | − |
| TLR7.1 | − | − | − | − | − | − | − | − |
| TLR7.2 | − | − | − | − | − | + | − | − |
| TLR7.3 | N/A | N/A | N/A | N/A | N/A | N/A | N/A | N/A |
| TLR7.4 | − | − | − | − | − | − | − | − |
| TLR8.1 | − | − | − | − | − | − | − | − |
| TLR8.2 | − | − | − | − | − | − | − | − |
| TLR8.4 | − | − | − | − | − | − | − | − |
| TLR9.1 | − | − | − | − | − | − | − | − |
| TLR9.2 | − | − | − | − | − | − | − | − |
| TLR10.1 | − | − | − | − | − | − | − | − |
| TLR10.2 | − | − | − | − | − | − | − | − |
| TLR10.3 | − | − | − | − | − | − | − | + |
| TLR10.4 | + | − | − | − | − | − | − | − |
| TLR10.5 | + | − | − | − | − | − | − | − |
| MDA5.1 | − | − | − | − | − | − | − | − |
| MDA5.2 | − | − | − | − | − | − | − | − |
| MDA5.3 | − | − | − | − | − | − | − | − |
| DDX58.1 | − | − | − | − | − | − | − | − |
| DDX58.2 | − | − | − | + | − | − | − | + |
| CARD4.0 | N/A | N/A | N/A | N/A | N/A | N/A | N/A | N/A |
| CARD4.1 | − | − | − | − | − | − | − | − |
| CARD4.2 | − | − | − | − | ++ | − | + | − |
| CARD4.3 | − | − | − | − | + | + | + | − |
| CARD15.1 | − | − | − | − | − | − | − | − |
| CARD15.2 | − | − | − | − | − | − | − | − |
| CARD15.3 | − | − | − | − | − | − | − | − |
| CARD15.4 | − | − | − | − | − | + | − | − |

*Designations in the SNP-column are as provided in table 3.
‘++’ indicates a highly significant association (p ≦ 0.01),
‘+’ indicates a significant association (0.01 < p ≦ p 0.05),
‘−’ indicates no significant association (p > 0.05), and
‘N/A’ indicates no available data.

1. A method for the prognosis of the development of an immune response to a biopharmaceutical treatment or diagnostic monoclonal antibody, in a subject, by the identification of one or more polymorphisms, such as SNPs, present in one or more pattern recognition receptor (PRR) genes, wherein the PRR polymorphisms are indicators for the likely prognosis of the development of an immune response to the biopharmaceutical or diagnostic.

2. A method for determining whether a subject is likely to benefit from the administration of a biopharmaceutical treatment or antibody diagnostic, by the identification of PRR polymorphisms, such as SNPs, present in one or more PRR genes, wherein the PRR polymorphisms are indicators for the likely prognosis of treatments with the biopharmaceutical or diagnostic.

3. A method for the prognosis of a treatment of a disease in a subject said treatment comprising the administration of a biopharmaceutical treatment to the subject, said method comprising the steps of:
a) Obtaining a sample comprising the genetic code from the subject;
b) Determining the presence or absence or copy number of at least 1 polymorphism, such as at least one single nucleotide polymorphism (SNP), in one or more PRR genes;
c) Comparing the presence or absence or copy number of the at least one polymorphism, such as at least one SNP, identified in step b) with control data obtained from either:
i) At least one subject which has been successfully treated for the disease using the biopharmaceutical (negative control); and/or,
ii) At least one subject which has developed the disease and has a history of failed treatment of said disease (positive control).

4. A method for determination of the suitability of using diagnostic antibody constructs specific for a disease epitope, for the in vivo detection of the disease in a subject, said method comprising the steps of:
a) Obtaining a sample comprising the genetic code from the subject
b) Determining the presence or absence or copy number of at least 1 polymorphism, such as at least one single nucleotide polymorphism (SNP), present in the genes for one or more PRR;
c) Comparing the presence or absence or copy number of the at least one polymorphism, such as at least one SNP, identified in step b) with control data obtained from either:
i) At least one subject which has developed an immune response to the biopharmaceutical; and/or (positive control),
ii) At least one subject which has not developed an immune response to the biopharmaceutical despite repeated administrations of the biopharmaceutical (negative control).

5. The method according to any one of claims 1-4, wherein the disease is selected form the group consisting of autoimmune diseases, infectious diseases, blood disorders, cancer, cardiovascular disease, diabetes and metabolic disorders, digestive disorders, eye conditions, genetic disorders, neurological disorders, respiratory disorders, skin disorders, transplantation rejection and graft-versus-host diseases.

6. The method according to claim 4, wherein the disease is a cancer.

7. The method according to any one of claims 1-3 wherein the disease is an inflammatory or autoimmune disease.

8. The method according to claim 7, wherein the disease is selected form the group consisting of: rheumatoid arthritis, psoriatic arthritis, ankylosing spondylitis, psoriasis, Crohn's disease, multiple sclerosis, and systemic lupus erythematosus.

9. The method according to claim 7 or 8, wherein the disease is a rheumatic disease, such as rheumatoid arthritis.

10. The method according to claim 7, wherein the disease is multiple sclerosis.

11. The method according to any one of claims 1-10, wherein the biopharmaceutical treatment comprises administering a biopharmaceutical agent to the subject.

12. The method according to claim 11, wherein the biopharmaceutical agent is a monoclonal antibody therapeutic.

13. The method according to claim 12, wherein the monoclonal antibody therapeutic is a chimeric monoclonal antibody.

14. The method according to claim 12, wherein the monoclonal antibody is a fully human antibody.

15. The method according to any one of claims 1-14 wherein the biopharmaceutical agent is a tumor necrosis factor-alpha (TNF-alpha) neutralising compound.

16. The method according to claim 15, wherein the biopharmaceutical agent is a TNF-alpha receptor antagonist, such as Etanercept.

17. The method according to claim 15, wherein the biopharmaceutical agent is a monoclonal antibody, such as Infliximab or Adalimumab.

18. The method according to claim 11, wherein the biopharmaceutical agent is an interferon, such as beta-interferon.

19. The method according to any one of claims 1-18, wherein the one or more polymorphisms is present in one or more PRR genes independently selected from the group consisting of the Toll-like receptors (TLR), the NOD-like receptors (NLR), and the retinoic acid-inducible gene I-like receptors (RLR).

20. The method according to any one of claims 1-19, wherein said one or more PRR genes are selected from the group consisting of TLR1, TLR2, TLR3, TLR4, TLR5, TLR6, TLR7, TLR8, TLR9, TLR 10, IFIH1 (MDA5), DDX58 (RIG-I), NOD1 (CARD4), and NOD2 (CARD15).

21. The method according to any one of claims 1-20, wherein the at least one SNP is selected from the group consisting of the SNPs shown in Table 2, table 3 or in table 2 of WO 2007/025989.

22. The method according to claim 20 or 21, wherein at least one of the SNPs is a SNP found in the genes for a TLR selected from the group consisting of TLR5, TLR7, TLR8 and TLR9.

23. The method according to claim 22, wherein at least one of the SNPs is a SNP found in the gene for TLR5.

24. The method according to claim 22 or 23, wherein at least one of the SNPs is selected from the group consisting of TLR5.3, TLR9.1, TLR7.1, and TLR8.1.

25. The method according to any one of claims 1-24, wherein step b) comprises determining the presence or absence of at least 2 SNPs in the genes for at least 2 independent PRRs.

26. The method according to claim 25, wherein step b) comprises determining the presence or absence of at least five SNPs in the genes for one or more PRRs.

27. The method according to claim 26, wherein the at least five SNPs are present in at least 3 independent PRR genes.

28. The method according to any one of claims 25-27, wherein step b) comprises determining the presence or absence of at least eight SNPs in the genes for at least three independent PRRs.

29. The method according to any one of claims 25-28, wherein the determining the presence or absence of at least 2 single nucleotide polymorphisms (SNP) referred to in step b) occurs concurrently.

30. The method according to claim 29 wherein step b) comprises of a multiplexed PCR reaction for the co-amplification of said at least two SNPs.

31. The method according to claim 29 or 30, where said at least 5, such as said at least 8 SNPs are detected or co-amplified concurrently.

32. The method according to claim 30 or 31, wherein step b) comprises the following sequential steps:
i) a multiplexed PCR reaction in which the SNPs are amplified,
ii) an allele-specific primer extension reaction (ASPE) in which label moieties are incorporated into the ASPE-primers which match the genotype of the sample,
iii) isolating the extension reaction products into separate population of individual SNP amplification products.

33. The method according to claim 32, wherein the labeled moiety referred to in step ii) is a biotin label, such as a biotinylated nucleotide.

34. The method according to claim 32 or 33, wherein step iii) comprises a hybridisation based isolation of individual populations of SNP amplification products, such as bead-array hybridisation.

35. The method according to any one of claims 1-34, wherein the heterozygosity or copy number of each SNPs is determined.

36. The method according to any one of claims 3-35, wherein the controlled data referred to in step c) is obtained by performing comparative SNP analysis on one or more subject groups selected from the subject groups consisting of:
i) One or more subjects which have developed the disease;
ii) One or more subjects which have developed the disease and have also history of failed treatment of said disease using the biopharmaceutical agent;
iii) One or more subjects which have not developed the disease;
iv) One or more subjects which have developed the disease but have shown a positive response to therapeutic treatment;
Wherein the comparative SNP analysis may be performed either prior to, concurrently or subsequent to step c).

37. A kit for use in the prognostic method according to any one of the preceding claims, said kit comprising:
i) A means for detecting at least one polymorphism, such as SNP, in the genes for one or more PRRs;
ii) A means for comparing the presence or absence of the at least one SNP identified in step i) with control data obtained from a subject which has developed the disease and has a history of failed treatment of said disease.

38. The kit according to claim 37, wherein the at least one polymorphism is present in one or more PRR genes independently selected from the group consisting of the Toll-like receptors (TLR), the NOD-like receptors (NLR), and the retinoic acid-inducible gene I-like receptors (RLR).

39. The kit according to any one of claim 37 or 38, which kit comprises at least one primer set, such as a primer set according to table 4 or 5; and optionally one or more elements selected from
i) a control sample, such as DNA-samples with known genotypes for the at least one polymorphic locus;
ii) instructions for use;
iii) a PCR-reagent mixture;
iv) a piece of software capable of performing data analysis; and
v) a biopharmaceutical according to the biopharmaceutical treatment.

40. The kit according to any one of claims 37-39, which kit comprises at least one polynucleotide comprising a nucleotide sequence corresponding to any one sequence of SEQ ID NO: 1-252.

41. A method of selecting the appropriate treatment or diagnostic method for an individual suffering from, or likely to develop a disease, comprising performing the method according to any one of claims 1-36.

42. A method for the identification of one or more polymorphisms of pattern recognition receptor genes which are correlated to a prognosis of a subject for the development of an immune response to a bio-agent, such as a biopharmaceutical or diagnostic monoclonal antibody, said method comprising the steps of:
a) Collecting genetic material or information from:
i) a population of subjects which have a history of successful treatment or diagnosis with the bio-agent; and
ii) a population of subjects which have a history of failed treatment or diagnosis with the bio-agent;
b) For each of the subjects, perform a series of genetic analysis to characterize the polymorphisms present in their PRR genes, preferably using a multiplex reaction;
c) Perform statistical analysis of the data obtained in b) to identify which polymorphisms are having a significant correlation to either population i) or pollution ii).

43. The method according to claim 42, wherein the one or more polymorphisms is present in one or more PRR genes independently selected from the group consisting of the Toll-like receptors (TLR), the NOD-like receptors (NLR), and the retinoic acid-inducible gene I-like receptors (RLR).
